SEQUENCE LISTING (222 sequences)

SEQ ID NO: 1 - DNA, 57 bp, synthetic construct
  source 1..57: mol_type = other DNA
atggattggatctggcggatcctgttccttgtgggagctgccacaggcgcccacagc

SEQ ID NO: 2 - DNA, 24 bp, synthetic construct
  source 1..24: mol_type = other DNA
gactacaaggacgacgacgacaag

SEQ ID NO: 3 - DNA, 117 bp, synthetic construct
  source 1..117: mol_type = other DNA
atcgccaggctggaggagaaggtgaagaccctgaaggcccagaacagcgagctggccagcaccgccaacatgctgagggagcaggtggcccagctgaagcagaaggtgatgaaccac

SEQ ID NO: 4 - DNA, 564 bp, synthetic construct
  source 1..564: mol_type = other DNA
ctgatcctgacactgagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 5 - DNA, 66 bp, synthetic construct
  source 1..66: mol_type = other DNA
ggcagcggcgccaccaactttagcctgctgaaacaagccggcgacgtggaagagaatcccggacct

SEQ ID NO: 6 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtccgcggcgaaggtgagggagatgccaccaacggcaagctgaccctgaagttcatctgcaccaccggcaagctgcccgtgccctggcccaccctcgtgaccaccttcggctacggcgtggcctgcttcagccgctaccccgaccacatgaagcagcacgacttcttcaagtccgccatgcccgaaggctacgtccaggagcgcaccatctctttcaaggacgacggtacctacaagacccgcgccgaggtgaagttcgagggcgacaccctggtgaaccgcatcgagctgaagggcatcgacttcaaggaggacggcaacatcctggggcacaagctggagtacaacttcaacagccactacgtctatatcacggccgacaagcagaagaactgcatcaaggctaacttcaagatccgccacaacgttgaggacggcagcgtgcagctcgccgaccactaccagcagaacacccccatcggcgacggccccgtgctgctgcccgacaaccactacctgagccatcagtccaagctgagcaaagaccccaacgagaagcgcgatcacatggtcctgctggagttcgtgaccgccgccgggattacacatggcatggacgagctgtacaagtaa

SEQ ID NO: 7 - protein, 19 aa, synthetic construct
MDWIWRILFLVGAATGAHS

SEQ ID NO: 8 - protein, 8 aa, synthetic construct
DYKDDDDK

SEQ ID NO: 9 - protein, 39 aa, synthetic construct
IARLEEKVKTLKAQNSELASTANMLREQVAQLKQKVMNH

SEQ ID NO: 10 - protein, 188 aa, synthetic construct
LILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 11 - protein, 22 aa, synthetic construct
GSGATNFSLLKQAGDVEENPGP

SEQ ID NO: 12 - protein, 239 aa, synthetic construct
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 13 - DNA, 561 bp, synthetic construct
  source 1..561: mol_type = other DNA
atcctgacactgagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 14 - protein, 187 aa, synthetic construct
ILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 15 - DNA, 555 bp, synthetic construct
  source 1..555: mol_type = other DNA
acactgagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 16 - protein, 185 aa, synthetic construct
TLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 17 - DNA, 549 bp, synthetic construct
  source 1..549: mol_type = other DNA
agcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 18 - protein, 183 aa, synthetic construct
SLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 19 - DNA, 1122 bp, synthetic construct
  source 1..1122: mol_type = other DNA
ttcccctggctgctgatcatcatcttcggcatctttggcctgaccgtgatgctgttcgtgttcctgttcagcaagcagcagcggatcaagatgctgatcctgcctcctgtgcctgtgcctaagatcaagggcatcgaccccgacctgctgaaagagggcaagctggaagaagtgaacaccatcctggccatccacgacagctacaagcccgagttccacagcgacgatagctgggtcgagttcatcgagctggacatcgacgagcccgacgagaaaaccgaggaaagcgacaccgacagactgctgagcagcgaccacgagaagtcccactctaacctgggcgtgaaggatggcgatagcggcagaacaagctgctgcgagcccgatatcctggaaaccgacttcaacgccaacgacatccacgagggcaccagcgaagttgcccagcctcaaagactgaagggcgaagccgatctgctgtgcctggaccagaagaaccagaacaacagcccctaccacgacgcctgtcctgctacacagcagcctagtgtgatccaggccgagaagaacaagccccagcctctgcctacagagggcgccgaatctacacatcaggccgctcacatccagctgagcaaccctagcagcctgagcaacatcgacttctacgcccaagtgtccgacatcacaccagccggatctgtggtgctgtctcccggccagaaaaacaaggccggcatgtcccagtgcgacatgcaccctgaaatggtgtccctgtgccaagagaacttcctgatggacaacgcctacttctgcgaggccgacgccaagaaatgcatccctgtggctccccacatcaaggtggaaagccacattcagcccagcctgaatcaagaggatatctacatcaccaccgagagcctgaccaccgctgctggtagacctggaacaggcgagcatgtgcctggctctgaaatgcccgtgcctgactacaccagcatccacatcgtgcagagccctcagggcctgatcctgaatgctacagctctgcccctgccagacaaagagttcctgtctagctgcggctacgtgtccaccgaccagctgaacaagatcatgccc

SEQ ID NO: 20 - protein, 374 aa, synthetic construct
FPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 21 - DNA, 933 bp, synthetic construct
  source 1..933: mol_type = other DNA
atcccctggctgggacatctgctcgttggactgtctggcgccttcggcttcatcatcctggtgtacctgctgatcaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 22 - protein, 311 aa, synthetic construct
IPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 23 - DNA, 936 bp, synthetic construct
  source 1..936: mol_type = other DNA
atcccctggctgggacatctgctcgttggactgtctggcgccttcggcttcatcatcctggtgtacctgctgatcgccaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 24 - protein, 312 aa, synthetic construct
IPWLGHLLVGLSGAFGFIILVYLLIANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 25 - DNA, 939 bp, synthetic construct
  source 1..939: mol_type = other DNA
atcccctggctgggacatctgctcgttggactgtctggcgccttcggcttcatcatcctggtgtacctgctgatcgccgctaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 26 - protein, 313 aa, synthetic construct
IPWLGHLLVGLSGAFGFIILVYLLIAANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 27 - DNA, 942 bp, synthetic construct
  source 1..942: mol_type = other DNA
atcccctggctgggacatctgctcgttggactgtctggcgccttcggcttcatcatcctggtgtacctgctgatcgccgctgccaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 28 - protein, 314 aa, synthetic construct
IPWLGHLLVGLSGAFGFIILVYLLIAAANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 29 - DNA, 945 bp, synthetic construct
  source 1..945: mol_type = other DNA
atcccctggctgggacatctgctcgttggactgtctggcgccttcggcttcatcatcctggtgtacctgctgatcgccgctgccgctaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 30 - protein, 315 aa, synthetic construct
IPWLGHLLVGLSGAFGFIILVYLLIAAAANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 31 - DNA, 948 bp, synthetic construct
  source 1..948: mol_type = other DNA
atcccctggctgggacatctgctcgttggactgtctggcgccttcggcttcatcatcctggtgtacctgctgatcgccgctgccgctgctaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 32 - protein, 316 aa, synthetic construct
IPWLGHLLVGLSGAFGFIILVYLLIAAAAANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 33 - DNA, 951 bp, synthetic construct
  source 1..951: mol_type = other DNA
atcccctggctgggacatctgctcgttggactgtctggcgccttcggcttcatcatcctggtgtacctgctgatcgccgctgccgctgctgccaactgccggaacacaggcccttggctgaagaaagtgctgaagtgcaacacccctgatccgagcaagttctttagccagctgagcagcgagcatggcggcgacgttcagaaatggctgtctagcccatttcctagcagcagcttcagccctggtggactggcccctgagattagccctctggaagtgctggaacgggacaaagtgacccagctgctcctgcagcaggataaggtgccagaacctgccagcctgtccagcaatcacagcctgaccagctgctttaccaaccagggctacttcttcttccatctgcctgacgctctcgagatcgaggcctgccaggtgtacttcacctacgatccctacagcgaggaagatcccgacgaaggcgttgcaggcgctcctacaggatcttctccacagcctctgcagccactgtccggcgaggatgatgcctactgcacctttccaagccgggacgacctgctgctgtttagccctagtctgctcggcggaccttctccaccatctacagctccaggtggaagcggagccggcgaagaaagaatgcctcctagcctgcaagagcgggtgcccagagattgggatcctcaacctctcggacctcctacaccaggcgtgccagacctggtggattttcagcctcctccagagctggtgctgagagaagctggcgaagaagtgccagacgccggacctagagagggcgttagctttccttggagcagacctcctggacagggcgagttcagagccctgaatgctagactgcccctgaacaccgatgcctacctgtctctgcaagaactgcagggacaagaccccacacacctcgtt

SEQ ID NO: 34 - protein, 317 aa, synthetic construct
IPWLGHLLVGLSGAFGFIILVYLLIAAAAAANCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 35 - DNA, 60 bp, synthetic construct
  source 1..60: mol_type = other DNA
ggcgtgctgctgtacatcctgctgggcacaatcggaacactggtggctgtgctggctgcc

SEQ ID NO: 36 - protein, 20 aa, synthetic construct
GVLLYILLGTIGTLVAVLAA

SEQ ID NO: 37 - DNA, 519 bp, synthetic construct
  source 1..519: mol_type = other DNA
ctcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

SEQ ID NO: 38 - protein, 173 aa, synthetic construct
LEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 39 - DNA, 60 bp, synthetic construct
  source 1..60: mol_type = other DNA
atgatcattgccgtgctgatcctggtggccgtcgtgtgtctggtcaccgtgtgcgtgatc

SEQ ID NO: 40 - protein, 20 aa, synthetic construct
MIIAVLILVAVVCLVTVCVI

SEQ ID NO: 41 - DNA, 117 bp, synthetic construct
  source 1..117: mol_type = other DNA
tgcccaagtcctttgtttccgggtcctagcaagccgttctgggtcctcgttgtcgtaggaggagtgcttgcgtgctatagtctccttgtaacggttgctttcattatcttttgggta

SEQ ID NO: 42 - protein, 39 aa, synthetic construct
CPSPLFPGPSKPFWVLVVVGGVLACYSLLVTVAFIIFWV

SEQ ID NO: 43 - DNA, 1050 bp, synthetic construct
  source 1..1050: mol_type = other DNA
aagcagcagcggatcaagatgctgatcctgcctcctgtgcctgtgcctaagatcaagggcatcgaccccgacctgctgaaagagggcaagctggaagaagtgaacaccatcctggccatccacgacagctacaagcccgagttccacagcgacgatagctgggtcgagttcatcgagctggacatcgacgagcccgacgagaaaaccgaggaaagcgacaccgacagactgctgagcagcgaccacgagaagtcccactctaacctgggcgtgaaggatggcgatagcggcagaacaagctgctgcgagcccgatatcctggaaaccgacttcaacgccaacgacatccacgagggcaccagcgaagttgcccagcctcaaagactgaagggcgaagccgatctgctgtgcctggaccagaagaaccagaacaacagcccctaccacgacgcctgtcctgctacacagcagcctagtgtgatccaggccgagaagaacaagccccagcctctgcctacagagggcgccgaatctacacatcaggccgctcacatccagctgagcaaccctagcagcctgagcaacatcgacttctacgcccaagtgtccgacatcacaccagccggatctgtggtgctgtctcccggccagaaaaacaaggccggcatgtcccagtgcgacatgcaccctgaaatggtgtccctgtgccaagagaacttcctgatggacaacgcctacttctgcgaggccgacgccaagaaatgcatccctgtggctccccacatcaaggtggaaagccacattcagcccagcctgaatcaagaggatatctacatcaccaccgagagcctgaccaccgctgctggtagacctggaacaggcgagcatgtgcctggctctgaaatgcccgtgcctgactacaccagcatccacatcgtgcagagccctcagggcctgatcctgaatgctacagctctgcccctgccagacaaagagttcctgtctagctgcggctacgtgtccaccgaccagctgaacaagatcatgccc

SEQ ID NO: 44 - protein, 350 aa, synthetic construct
KQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 45 - DNA, 495 bp, synthetic construct
  source 1..495: mol_type = other DNA
catagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 46 - protein, 165 aa, synthetic construct
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 47 - protein, 226 aa, synthetic construct
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 48 - protein, 23 aa, synthetic construct
LILTLSLILVVILVLLTVLALLS

SEQ ID NO: 49 - protein, 235 aa, synthetic construct
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 50 - protein, 273 aa, synthetic construct
WSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 51 - protein, 246 aa, synthetic construct
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFY

SEQ ID NO: 52 - protein, 24 aa, synthetic construct
FPWLLIIIFGIFGLTVMLFVFLFS

SEQ ID NO: 53 - DNA, 69 bp, synthetic construct
  source 1..69: mol_type = other DNA
ccctggctgctgatcatcatcttcggcatctttggcctgaccgtgatgctgttcgtgttcctgttcagc

SEQ ID NO: 54 - protein, 389 aa, synthetic construct
TLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 55 - protein, 98 aa, synthetic construct
MSYYHHHHHHLESTSLYKKAGSEFDLERNRAAASRCRQKRKVWVQSLEKKAEDLSSLNGQLQSEVTLLRNEVAQLKQLLLAHKDCPVTAMQKKSGFLE

SEQ ID NO: 56 - protein, 80 aa, synthetic construct
MSYYHHHHHHLESTSLYKKAGSEFKRKLWVSSLEKKAEELTSQNIQLSNEVTLLRNEVAQLKQLLLAHKDCPVTALQKLE

SEQ ID NO: 57 - protein, 82 aa, synthetic construct
MSYYHHHHHHLESTSLYKKAGSEFQRKVWVMSLEKKAEELTQTNMQLQNEVSMLKNEVAQLKQLLLTHKDCPITAMQKESLE

SEQ ID NO: 58 - protein, 85 aa, synthetic construct
MSYYHHHHHHLESTSLYKKAGSEFACQSRKKKKEYMLGLEARLKAALSENEQLKKENGTLKRQLDEVVSENQRLKVPSPKRRVLE

SEQ ID NO: 59 - protein, 49 aa, synthetic construct
EYLQGLEARLQAVLADNQQLRRENAALRRRLEALLAENSELKLGSGNRK

SEQ ID NO: 60 - protein, 85 aa, synthetic construct
MSYYHHHHHHLESTSLYKKAGSEFCRLNRLKKKEYVMGLESRVRGLAAENQELRAENRELGKRVQALQEESRYLRAVLANETGLE

SEQ ID NO: 61 - protein, 68 aa, synthetic construct
MSYYHHHHHHLESTSLYKKAGSGSQKADTLHLESEDLEKQNAALRKEIKQLTEELKYFTSVLNSHELE

SEQ ID NO: 62 - protein, 89 aa, synthetic construct
MSYYHHHHHHLESTSLYKKAGSEFRSRKKQTQKADKLHEEYESLEQENTMLRREIGKLTEELKHLTEALKEHEKMCPLLLCPMNFVHLE

SEQ ID NO: 63 - protein, 42 aa, synthetic construct
DKADALHQQHESLEKDNLALRKEIQSLQAELAWWSRTLHVHE

SEQ ID NO: 64 - protein, 67 aa, synthetic construct
MSYYHHHHHHLESTSLYKKAGSGSQRNVETQQKVLELTSDNDRLRKRVEQLSRELDTLRGIFRQLLE

SEQ ID NO: 65 - protein, 99 aa, synthetic construct
MSYYHHHHHHLESTSLYKKAGSEFSDEYKIRRERNNIAVRKSRDKAKMRNLETQHKVLELTAENERLQKKVEQLSRELSTLRNLFKQLPEPLLASSGHC

SEQ ID NO: 66 - protein, 103 aa, synthetic construct
MSYYHHHHHHLESTSLYKKAGSEFGERNNMAVKKSRLKSKQKAQDTLQRVNQLKEENERLEAKIKLLTKELSVLKDLFLEHAHNLADNVQSISTENTTADGLE

SEQ ID NO: 67 - protein, 67 aa, synthetic construct
MSYYHHHHHHLESTSLYKKAGSGSRRNQEMQQKLVELSAENEKLHQRVEQLTRDLAGLRQFFKQLLE

SEQ ID NO: 68 - protein, 100 aa, synthetic construct
MSYYHHHHHHLESTSLYKKAGSEFHLEYRLRRERNNIAVRKSRDKAKRRILETQQKVLEYMAENERLRSRVEQLTQELDTLRNLFRQIPEAANLIKGVLE

SEQ ID NO: 69 - protein, 75 aa, synthetic construct
MSYYHHHHHHLESTSLYKKAGSEFRMKEKEQENERKVAQLAEENERLKQEIERLTREVEATRRALIDRMVNLHQA

SEQ ID NO: 70 - protein, 84 aa, synthetic construct
MSYYHHHHHHLESTSLYKKAGSEFQRKLETIVQLERELERLTNERERLLRARGEADRTLEVMRQQLTELYRDIFQHLRDESGNS

SEQ ID NO: 71 - protein, 85 aa, synthetic construct
MSYYHHHHHHLESTSLYKKAGSEFDPQLKREIRLMKNREAARECRRKKKEYVKCLENRVAVLENQNKTLIEELKTLKDLYSNKSV

SEQ ID NO: 72 - protein, 87 aa, synthetic construct
MSYYHHHHHHLESTSLYKKAGSGSKEEATRKRELRLMKNREAARECRRKKKEYVKCLENRVAVLENQNKTLIEELKALKDLYCHKVE

SEQ ID NO: 73 - protein, 86 aa, synthetic construct
MSYYHHHHHHLESTSLYKKAGSGSKEEATRKRELRLMKNREAARECRRKKKEYVKCLENRVAVLENQNKTLIEELKALRDLYCQLE

SEQ ID NO: 74 - protein, 58 aa, synthetic construct
MSYYHHHHHHLESTSLYKKAGSGSEYVKCLESRVAVLEVQNKKLIEELETLKDICSLE

SEQ ID NO: 75 - protein, 86 aa, synthetic construct
MSYYHHHHHHLESTSLYKKAGSGSKEAARKREVRLMKNREAARECRRKKKEYVKCLENRVAVLENQNKTLIEELKALKDLYCHKSD

SEQ ID NO: 76 - protein, 94 aa, synthetic construct
MSYYHHHHHHLESTSLYKKAGSEFEKRRLNDLVLENKLIALGEENATLKAELLSLKLKFGLISSTAYAQEIQKLSNSTAVYFQDYQTSKSNVLE

SEQ ID NO: 77 - protein, 37 aa, synthetic construct
LNDAAIAGRLAALMEENALLKGELKALKLRFGLLPLT

SEQ ID NO: 78 - protein, 84 aa, synthetic construct
MSYYHHHHHHLESTSLYKKAGSEFQRKLERIARLEEKVKTLKAQNSELASTANMLREQVAQLKQKVMNHVNSGCQLMLTQQYLE

SEQ ID NO: 79 - protein, 90 aa, synthetic construct
MSYYHHHHHHLESTSLYKKAGSEFQRVQQRHVLESEKNQLLQQVDHLKQEISRLVRERDAYKEKYEKLVSSGFRENGSSSDNPSSPEFFM

SEQ ID NO: 80 - protein, 92 aa, synthetic construct
MSYYHHHHHHLESTSLYKKAGSEFQYKRVQQKHHLENEKTQLIQQVEQLKQEVSRLARERDAYKVKCEKLANSGFREAGSTSDSPSSPEFFL

SEQ ID NO: 81 - protein, 46 aa, synthetic construct
QQRHILESEKCQLQSQVEQLKLEVGRLAKERDLYKEKYEKLAGRGG

SEQ ID NO: 82 - protein, 45 aa, synthetic construct
QQRRGLEAERARLAAQLDALRAEVARLARERDLYKARCDRLTSSG

SEQ ID NO: 83 - protein, 77 aa, synthetic construct
MSYYHHHHHHLESTSLYKKAGSGSEYVECLEKKVETFTSENNELWKKVETLENANRTLLQQLQKLQTLVTNKISRLE

SEQ ID NO: 84 - protein, 76 aa, synthetic construct
MSYYHHHHHHLESTSLYKKAGSGSEYIDGLETRMSACTAQNQELQRKVLHLEKQNLSLLEQLKKLQAIVVQSTSLE

SEQ ID NO: 85 - protein, 100 aa, synthetic construct
MSYYHHHHHHLESTSLYKKAGSEFWRRKIRNKRSAQESRRKKKVYVGGLESRVLKYTAQNMELQNKVQLLEEQNLSLLDQLRKLQAMVIEISNKTSSRLE

SEQ ID NO: 86 - protein, 104 aa, synthetic construct
MSYYHHHHHHLESTSLYKKAGSEFWRKIRNKQSAQDSRRRKKEYIDGLESRVAACSAQNQELQKKVQELERHNISLVAQLRQLQTLIAQTSNKAAQTSTCVLLE

SEQ ID NO: 87 - protein, 39 aa, synthetic construct
EYMDSLEDDVESCSTENLELRKKVEVLENTNRASHGELV

SEQ ID NO: 88 - protein, 99 aa, synthetic construct
MSYYHHHHHHLESTSLYKKAGSGSEEQKDEKYWSRRYKNNEAAKRSRDARRLKENQISVRAAFLEKENALLRQEVVAVRQELSHYRAVLSRYQAQHGAL

SEQ ID NO: 89 - protein, 71 aa, synthetic construct
MSYYHHHHHHLESTSLYKKAGSGRLKENQIAIRASFLEKENSALRQEVADLRKELGKCKNILAKYEARHLE

SEQ ID NO: 90 - protein, 92 aa, synthetic construct
MSYYHHHHHHLESTSLYKKAGSEFNWTRRKKNNVAAKRSRDARRLKENQITIRAAFLEKENTALRTEVAELRKEVGKCKTIVSKYETKYGPL

SEQ ID NO: 91 - protein, 115 aa, synthetic construct
MSYYHHHHHHLESTSLYKKAGSEFGVTQKEELEKQKAELQQEVEKLASENASMKLELDALRSKYEALQTFARTVARSPVAPARGPLAAGLGPLVPGKVAATSVITIVKSKTDALE

SEQ ID NO: 92 - protein, 76 aa, synthetic construct
MSYYHHHHHHLESTSLYKKAGSGSTQKEELERQRVELQQEVEKLARENSSMRLELDALRSKYEALQTFARTVARLE

SEQ ID NO: 93 - protein, 53 aa, synthetic construct
CQKEELQKQKSELEREVDKLARENAAMRLELDALRGKCEALQGFARSVAAARG

SEQ ID NO: 94 - protein, 97 aa, synthetic construct
MSYYHHHHHHLESTSLYKKAGSEFQTARDRKKARMSELEQQVVDLEEENQKLLLENQLLREKTHGLVVENQELRQRLGMDALVAEEEAEAKGNEVLE

SEQ ID NO: 95 - protein, 96 aa, synthetic construct
MSYYHHHHHHLESTSLYKKAGSEFPSQNAFRKRKLERLEELEKKEAQLTVTNDQIHILKKENELLHFMLRSLLTERNMPCSSNTQLSCTKWLIRGC

SEQ ID NO: 96 - protein, 78 aa, synthetic construct
MSYYHHHHHHLESTSLYKKAGSEFKRAAQNRSAQKAFRQRREKYIKNLEEKSKLFDGLMKENSELKKMIESLKSKLKE

SEQ ID NO: 97 - protein, 132 aa, synthetic construct
MSYYHHHHHHLESTSLYKKAGFDDEDTPPNPKKEIEFPGGSPGRKRIDSEAKSRRTAQNRAAQRAFRDRKEAKMKSLQERVELLEQKDAQNKTTTDFLLCSLKSLLSEITKYRAKNSDDERILAFLDDLHLE

SEQ ID NO: 98 - protein, 109 aa, synthetic construct
MSYYHHHHHHLESTSLYKKAGFDDEDTPPNPKKEIEFQAKKKAQNRAAQKAFRERKEARMKELQDKLLESERNRQSLLKEIEELRKANTEINAENRLLLRSGNSTCRDL

SEQ ID NO: 99 - protein, 125 aa, synthetic construct
MSYYHHHHHHLESTSLYKKAGFDDEDTPPNPKKEIEFPKRQNRDAQRAYRERKNNKLQVLEETIESLSKVVKNYETKLNRLQNELQAKESENHALKQKLETLTLKQASVPAQDPILQNFSTCRDL

SEQ ID NO: 100 - protein, 121 aa, synthetic construct
MSYYHHHHHHLESTSLYKKAGSEFQQNRDAQRAYRERRTTRIQVLEEKVEMLHNLVDDWQRKYKLLESEFSDTKENLQKSIALNNELQKALPLIVNTPFQQQPENSSNTQLSCTKWLIRGC

SEQ ID NO: 101 - protein, 102 aa, synthetic construct
MSYYHHHHHHLESTSLYKKAGFDDEDTPPNPKKEIEFHPAALKRARNTEAARRSRARKLQRMKQLEDKVEELLSKNYHLENEVARLKKLVGDAARPAEIYES

SEQ ID NO: 102 - protein, 106 aa, synthetic construct
MSYYHHHHHHLESTSLYKKAGFDDEDTPPNPKKEIEFDDDEDLLIQSKKSHQKKKLKEKELESSIHELTEIAASLQKRIHTLETENKLLKNLVLSSGETFSTCRDL

SEQ ID NO: 103 - DNA, 69 bp, synthetic construct
  source 1..69: mol_type = other DNA
ctgatcctgacactgagcctgatcctggtggtcatcctggtgctgctgacagtgctggccctgctgtct

SEQ ID NO: 104 - protein, 22 aa, synthetic construct
ILTLSLILVVILVLLTVLALLS

SEQ ID NO: 105 - DNA, 66 bp, synthetic construct
  source 1..66: mol_type = other DNA
atcctgacactgagcctgatcctggtggtcatcctggtgctgctgacagtgctggccctgctgtct

SEQ ID NO: 106 - protein, 20 aa, synthetic construct
TLSLILVVILVLLTVLALLS

SEQ ID NO: 107 - DNA, 60 bp, synthetic construct
  source 1..60: mol_type = other DNA
acactgagcctgatcctggtggtcatcctggtgctgctgacagtgctggccctgctgtct

SEQ ID NO: 108 - protein, 18 aa, synthetic construct
SLILVVILVLLTVLALLS

SEQ ID NO: 109 - DNA, 54 bp, synthetic construct
  source 1..54: mol_type = other DNA
agcctgatcctggtggtcatcctggtgctgctgacagtgctggccctgctgtct

SEQ ID NO: 110 - DNA, 72 bp, synthetic construct
  source 1..72: mol_type = other DNA
ttcccctggctgctgatcatcatcttcggcatctttggcctgaccgtgatgctgttcgtgttcctgttcagc

SEQ ID NO: 111 - DNA, 681 bp, synthetic construct
  source 1..681: mol_type = other DNA
atcgccaggctggaggagaaggtgaagaccctgaaggcccagaacagcgagctggccagcaccgccaacatgctgagggagcaggtggcccagctgaagcagaaggtgatgaaccacctgatcctgacactgagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 112 - protein, 227 aa, synthetic construct
IARLEEKVKTLKAQNSELASTANMLREQVAQLKQKVMNHLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 113 - DNA, 678 bp, synthetic construct
  source 1..678: mol_type = other DNA
atcgccaggctggaggagaaggtgaagaccctgaaggcccagaacagcgagctggccagcaccgccaacatgctgagggagcaggtggcccagctgaagcagaaggtgatgaaccacatcctgacactgagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 114 - protein, 226 aa, synthetic construct
IARLEEKVKTLKAQNSELASTANMLREQVAQLKQKVMNHILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 115 - DNA, 672 bp, synthetic construct
  source 1..672: mol_type = other DNA
atcgccaggctggaggagaaggtgaagaccctgaaggcccagaacagcgagctggccagcaccgccaacatgctgagggagcaggtggcccagctgaagcagaaggtgatgaaccacacactgagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 116 - protein, 224 aa, synthetic construct
IARLEEKVKTLKAQNSELASTANMLREQVAQLKQKVMNHTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 117 - DNA, 666 bp, synthetic construct
  source 1..666: mol_type = other DNA
atcgccaggctggaggagaaggtgaagaccctgaaggcccagaacagcgagctggccagcaccgccaacatgctgagggagcaggtggcccagctgaagcagaaggtgatgaaccacagcctgatcctggtggtcatcctggtgctgctgacagtgctggccctgctgtctcatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 118 - protein, 222 aa, synthetic construct
IARLEEKVKTLKAQNSELASTANMLREQVAQLKQKVMNHSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 119 - DNA, 1239 bp, synthetic construct
  source 1..1239: mol_type = other DNA
atcgccaggctggaggagaaggtgaagaccctgaaggcccagaacagcgagctggccagcaccgccaacatgctgagggagcaggtggcccagctgaagcagaaggtgatgaaccacttcccctggctgctgatcatcatcttcggcatctttggcctgaccgtgatgctgttcgtgttcctgttcagcaagcagcagcggatcaagatgctgatcctgcctcctgtgcctgtgcctaagatcaagggcatcgaccccgacctgctgaaagagggcaagctggaagaagtgaacaccatcctggccatccacgacagctacaagcccgagttccacagcgacgatagctgggtcgagttcatcgagctggacatcgacgagcccgacgagaaaaccgaggaaagcgacaccgacagactgctgagcagcgaccacgagaagtcccactctaacctgggcgtgaaggatggcgatagcggcagaacaagctgctgcgagcccgatatcctggaaaccgacttcaacgccaacgacatccacgagggcaccagcgaagttgcccagcctcaaagactgaagggcgaagccgatctgctgtgcctggaccagaagaaccagaacaacagcccctaccacgacgcctgtcctgctacacagcagcctagtgtgatccaggccgagaagaacaagccccagcctctgcctacagagggcgccgaatctacacatcaggccgctcacatccagctgagcaaccctagcagcctgagcaacatcgacttctacgcccaagtgtccgacatcacaccagccggatctgtggtgctgtctcccggccagaaaaacaaggccggcatgtcccagtgcgacatgcaccctgaaatggtgtccctgtgccaagagaacttcctgatggacaacgcctacttctgcgaggccgacgccaagaaatgcatccctgtggctccccacatcaaggtggaaagccacattcagcccagcctgaatcaagaggatatctacatcaccaccgagagcctgaccaccgctgctggtagacctggaacaggcgagcatgtgcctggctctgaaatgcccgtgcctgactacaccagcatccacatcgtgcagagccctcagggcctgatcctgaatgctacagctctgcccctgccagacaaagagttcctgtctagctgcggctacgtgtccaccgaccagctgaacaagatcatgccc

SEQ ID NO: 120 - protein, 413 aa, synthetic construct
IARLEEKVKTLKAQNSELASTANMLREQVAQLKQKVMNHFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 121 - DNA, 696 bp, synthetic construct
  source 1..696: mol_type = other DNA
atcgccaggctggaggagaaggtgaagaccctgaaggcccagaacagcgagctggccagcaccgccaacatgctgagggagcaggtggcccagctgaagcagaaggtgatgaaccacatgatcattgccgtgctgatcctggtggccgtcgtgtgtctggtcaccgtgtgcgtgatcctcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

SEQ ID NO: 122 - protein, 232 aa, synthetic construct
IARLEEKVKTLKAQNSELASTANMLREQVAQLKQKVMNHMIIAVLILVAVVCLVTVCVILEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 123 - DNA, 696 bp, synthetic construct
  source 1..696: mol_type = other DNA
atcgccaggctggaggagaaggtgaagaccctgaaggcccagaacagcgagctggccagcaccgccaacatgctgagggagcaggtggcccagctgaagcagaaggtgatgaaccacggcgtgctgctgtacatcctgctgggcacaatcggaacactggtggctgtgctggctgccctcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

SEQ ID NO: 124 - protein, 232 aa, synthetic construct
IARLEEKVKTLKAQNSELASTANMLREQVAQLKQKVMNHGVLLYILLGTIGTLVAVLAALEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 125 - DNA, 753 bp, synthetic construct
  source 1..753: mol_type = other DNA
atcgccaggctggaggagaaggtgaagaccctgaaggcccagaacagcgagctggccagcaccgccaacatgctgagggagcaggtggcccagctgaagcagaaggtgatgaaccactgcccaagtcctttgtttccgggtcctagcaagccgttctgggtcctcgttgtcgtaggaggagtgcttgcgtgctatagtctccttgtaacggttgctttcattatcttttgggtactcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

SEQ ID NO: 126 - protein, 251 aa, synthetic construct
IARLEEKVKTLKAQNSELASTANMLREQVAQLKQKVMNHCPSPLFPGPSKPFWVLVVVGGVLACYSLLVTVAFIIFWVLEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 127 - DNA, 63 bp, synthetic construct
  source 1..63: mol_type = other DNA
ctgacactgagcctgatcctggtggtcatcctggtgctgctgacagtgctggccctgctgtct

SEQ ID NO: 128 - protein, 21 aa, synthetic construct
LTLSLILVVILVLLTVLALLS

SEQ ID NO: 129 - DNA, 57 bp, synthetic construct
  source 1..57: mol_type = other DNA
ctgagcctgatcctggtggtcatcctggtgctgctgacagtgctggccctgctgtct

SEQ ID NO: 130 - protein, 19 aa, synthetic construct
LSLILVVILVLLTVLALLS

SEQ ID NO: 131 - DNA, 51 bp, synthetic construct
  source 1..51: mol_type = other DNA
ctgatcctggtggtcatcctggtgctgctgacagtgctggccctgctgtct

SEQ ID NO: 132 - protein, 17 aa, synthetic construct
LILVVILVLLTVLALLS

SEQ ID NO: 133 - DNA, 72 bp, synthetic construct; identical to SEQ ID NO: 110
  source 1..72: mol_type = other DNA

SEQ ID NO: 134 - protein, 23 aa, synthetic construct
PWLLIIIFGIFGLTVMLFVFLFS

SEQ ID NO: 135 - DNA, 69 bp, synthetic construct; identical to SEQ ID NO: 53
  source 1..69: mol_type = other DNA

SEQ ID NO: 136 - protein, 22 aa, synthetic construct
WLLIIIFGIFGLTVMLFVFLFS

SEQ ID NO: 137 - DNA, 66 bp, synthetic construct
  source 1..66: mol_type = other DNA
tggctgctgatcatcatcttcggcatctttggcctgaccgtgatgctgttcgtgttcctgttcagc

SEQ ID NO: 138 - protein, 21 aa, synthetic construct
LLIIIFGIFGLTVMLFVFLFS

SEQ ID NO: 139 - DNA, 63 bp, synthetic construct
  source 1..63: mol_type = other DNA
ctgctgatcatcatcttcggcatctttggcctgaccgtgatgctgttcgtgttcctgttcagc

SEQ ID NO: 140 - protein, 20 aa, synthetic construct
LIIIFGIFGLTVMLFVFLFS

SEQ ID NO: 141 - DNA, 60 bp, synthetic construct
  source 1..60: mol_type = other DNA
ctgatcatcatcttcggcatctttggcctgaccgtgatgctgttcgtgttcctgttcagc

SEQ ID NO: 142 - protein, 19 aa, synthetic construct
IIIFGIFGLTVMLFVFLFS

SEQ ID NO: 143 - DNA, 57 bp, synthetic construct
  source 1..57: mol_type = other DNA
atcatcatcttcggcatctttggcctgaccgtgatgctgttcgtgttcctgttcagc

SEQ ID NO: 144 - protein, 18 aa, synthetic construct
IIFGIFGLTVMLFVFLFS

SEQ ID NO: 145 - DNA, 54 bp, synthetic construct
  source 1..54: mol_type = other DNA
atcatcttcggcatctttggcctgaccgtgatgctgttcgtgttcctgttcagc

SEQ ID NO: 146 - protein, 582 aa, synthetic construct
IARLEEKVKTLKAQNSELASTANMLREQVAQLKQKVMNHGVLLYILLGTIGTLVAVLAALEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 147 - DNA, 1746 bp, synthetic construct
  source 1..1746: mol_type = other DNA
atcgccaggctggaggagaaggtgaagaccctgaaggcccagaacagcgagctggccagcaccgccaacatgctgagggagcaggtggcccagctgaagcagaaggtgatgaaccacggcgtgctgctgtacatcctgctgggcacaatcggaacactggtggctgtgctggctgccctcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatcaagcagcagcggatcaagatgctgatcctgcctcctgtgcctgtgcctaagatcaagggcatcgaccccgacctgctgaaagagggcaagctggaagaagtgaacaccatcctggccatccacgacagctacaagcccgagttccacagcgacgatagctgggtcgagttcatcgagctggacatcgacgagcccgacgagaaaaccgaggaaagcgacaccgacagactgctgagcagcgaccacgagaagtcccactctaacctgggcgtgaaggatggcgatagcggcagaacaagctgctgcgagcccgatatcctggaaaccgacttcaacgccaacgacatccacgagggcaccagcgaagttgcccagcctcaaagactgaagggcgaagccgatctgctgtgcctggaccagaagaaccagaacaacagcccctaccacgacgcctgtcctgctacacagcagcctagtgtgatccaggccgagaagaacaagccccagcctctgcctacagagggcgccgaatctacacatcaggccgctcacatccagctgagcaaccctagcagcctgagcaacatcgacttctacgcccaagtgtccgacatcacaccagccggatctgtggtgctgtctcccggccagaaaaacaaggccggcatgtcccagtgcgacatgcaccctgaaatggtgtccctgtgccaagagaacttcctgatggacaacgcctacttctgcgaggccgacgccaagaaatgcatccctgtggctccccacatcaaggtggaaagccacattcagcccagcctgaatcaagaggatatctacatcaccaccgagagcctgaccaccgctgctggtagacctggaacaggcgagcatgtgcctggctctgaaatgcccgtgcctgactacaccagcatccacatcgtgcagagccctcagggcctgatcctgaatgctacagctctgcccctgccagacaaagagttcctgtctagctgcggctacgtgtccaccgaccagctgaacaagatcatgccc

SEQ ID NO: 148 - protein, 397 aa, synthetic construct
IARLEEKVKTLKAQNSELASTANMLREQVAQLKQKVMNHGVLLYILLGTIGTLVAVLAALEAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRP

SEQ ID NO: 149 - DNA, 1191 bp, synthetic construct
  source 1..1191: mol_type = other DNA
atcgccaggctggaggagaaggtgaagaccctgaaggcccagaacagcgagctggccagcaccgccaacatgctgagggagcaggtggcccagctgaagcagaaggtgatgaaccacggcgtgctgctgtacatcctgctgggcacaatcggaacactggtggctgtgctggctgccctcgaggcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatccatagaagggccctgaagcagaagatctggcctggcatcccatctccagagagcgagttcgagggcctgttcaccacacacaagggcaacttccagctgtggctgtaccagaacgatggctgcctttggtggtccccttgcacaccctttaccgaggatccaccagccagcctggaagtgctgagcgagagatgttggggcacaatgcaggccgtggaacccggcacagatgatgaaggacctctgctggaacctgtgggctctgaacatgcccaggacacctatctggtgctggacaagtggctgctgccccggaatcctccatctgaggatttgcctggacctggcggctccgtggatatcgtggctatggatgagggcagcgaggccagctcttgttcttctgccctggccagcaagccttctccagaaggcgcttctgccgccagcttcgagtacaccattctggaccctagcagccagctgctgaggcct

SEQ ID NO: 150 - protein, 5 aa, synthetic construct
GGGGS

SEQ ID NO: 151 - protein, 15 aa, synthetic construct
GGGGSGGGGSGGGGS

SEQ ID NO: 152 - DNA, 45 bp, synthetic construct
  source 1..45: mol_type = other DNA
ggtggcggaggttctggcggaggcggctctggtggtggtggatct

SEQ ID NO: 153 - DNA, 45 bp, synthetic construct
  source 1..45: mol_type = other DNA
ggtggcggaggttctggcggaggcggaagtggtggtggcggatct

SEQ ID NO: 154 - DNA, 705 bp, synthetic construct
  source 1..705: mol_type = other DNA
catcgccgcgcgctgaaacagaaaatttggccgggcattccgagcccggaaagcgaatttgaaggcctgtttaccacccataaaggcaactttcagctgtggctgtatcagaacgatggctgcctgtggtggagcccgtgcaccccgtttaccgaagatccgccggcgagcctggaagtgctgagcgaacgctgctggggcaccatgcaggcggtggaaccgggcaccgatgatgaaggcccgctgctggaaccggtgggcagcgaacatgcgcaggatacctatctggtgctggataaatggctgctgccgcgcaacccgccgagcgaagatctgccgggcccgggcggcagcgtggatattgtggcgatggatgaaggcagcgaagcgagcagctgcagcagcgcgctggcgagcaaaccgagcccggaaggcgcgagcgcggcgagctttgaatataccattctggatccgagcagccagctgctgcgcccgtggaccctgtgcccggaactgccgccgaccccgccgcatctgaaatatctgtatctggtggtgagcgatagcggcattagcaccgattatagcagcggcgatagccagggcgcgcagggcggcctgagcgatggcccgtatagcaacccgtatgaaaacagcctgattccggcggcggaaccgctgccgccgagctatgtggcgtgcagc

SEQ ID NO: 155 - protein, 19 aa, synthetic construct
AEGRGSLLTCGDVEENPGP

SEQ ID NO: 156 - protein, 18 aa, synthetic construct
EGRGSLLTCGDVEENPGP

SEQ ID NO: 157 - protein, 21 aa, synthetic construct
GSGEGRGSLLTCGDVEENPGP

SEQ ID NO: 158 - protein, 63 aa, synthetic construct
GSGSRVTELLYRMKRAETYCPRPLLAIHPTEARHKQKIVAPVKQLLNFDLLKLAGDVESNPGP

SEQ ID NO: 159 - protein, 19 aa, synthetic construct
LLCFLLLLLSGDVELNPGP

SEQ ID NO: 160 - protein, 20 aa, synthetic construct
HHFMFLLLLLAGDIELNPGP

SEQ ID NO: 161 - protein, 20 aa, synthetic construct
WFLVLLSFILSGDIEVNPGP

SEQ ID NO: 162 - protein, 20 aa, synthetic construct
KNCAMYMLLLSGDVETNPGP

SEQ ID NO: 163 - protein, 20 aa, synthetic construct
MVISQLMLKLAGDVEENPGP

SEQ ID NO: 164 - protein, 23 aa, synthetic construct
GSGQCTNYALLKLAGDVESNPGP

SEQ ID NO: 165 - protein, 8 aa, synthetic construct
  VARIANT 2: note = Any amino acid
  VARIANT 4: note = Any amino acid
DXEXNPGP

SEQ ID NO: 166 - protein, 71 aa, synthetic construct
DLERNRAAASRCRQKRKVWVQSLEKKAEDLSSLNGQLQSEVTLLRNEVAQLKQLLLAHKDCPVTAMQKKSG

SEQ ID NO: 167 - protein, 54 aa, synthetic construct
KRKLWVSSLEKKAEELTSQNIQLSNEVTLLRNEVAQLKQLLLAHKDCPVTALQK

SEQ ID NO: 168 - protein, 56 aa, synthetic construct
QRKVWVMSLEKKAEELTQTNMQLQNEVSMLKNEVAQLKQLLLTHKDCPITAMQKES

SEQ ID NO: 169 - protein, 59 aa, synthetic construct
ACQSRKKKKEYMLGLEARLKAALSENEQLKKENGTLKRQLDEVVSENQRLKVPSPKRRV

SEQ ID NO: 170 - protein, 60 aa, synthetic construct
CRLNRLKKKEYVMGLESRVRGLAAENQELRAENRELGKRVQALQEESRYLRAVLANETGL

SEQ ID NO: 171 - protein, 42 aa, synthetic construct
QKADTLHLESEDLEKQNAALRKEIKQLTEELKYFTSVLNSHE

SEQ ID NO: 172 - protein, 63 aa, synthetic construct
RSRKKQTQKADKLHEEYESLEQENTMLRREIGKLTEELKHLTEALKEHEKMCPLLLCPMNFVH

SEQ ID NO: 173 - protein, 41 aa, synthetic construct
QRNVETQQKVLELTSDNDRLRKRVEQLSRELDTLRGIFRQL

SEQ ID NO: 174 - protein, 75 aa, synthetic construct
SDEYKIRRERNNIAVRKSRDKAKMRNLETQHKVLELTAENERLQKKVEQLSRELSTLRNLFKQLPEPLLASSGHC

SEQ ID NO: 175 - protein, 77 aa, synthetic construct
GERNNMAVKKSRLKSKQKAQDTLQRVNQLKEENERLEAKIKLLTKELSVLKDLFLEHAHNLADNVQSISTENTTADG

SEQ ID NO: 176 - protein, 41 aa, synthetic construct
RRNQEMQQKLVELSAENEKLHQRVEQLTRDLAGLRQFFKQL

SEQ ID NO: 177 - protein, 74 aa, synthetic construct
HLEYRLRRERNNIAVRKSRDKAKRRILETQQKVLEYMAENERLRSRVEQLTQELDTLRNLFRQIPEAANLIKGV

SEQ ID NO: 178 - protein, 51 aa, synthetic construct
RMKEKEQENERKVAQLAEENERLKQEIERLTREVEATRRALIDRMVNLHQA

SEQ ID NO: 179 - protein, 60 aa, synthetic construct
QRKLETIVQLERELERLTNERERLLRARGEADRTLEVMRQQLTELYRDIFQHLRDESGNS

SEQ ID NO: 180 - protein, 61 aa, synthetic construct
DPQLKREIRLMKNREAARECRRKKKEYVKCLENRVAVLENQNKTLIEELKTLKDLYSNKSV

SEQ ID NO: 181 - protein, 63 aa, synthetic construct
KEEATRKRELRLMKNREAARECRRKKKEYVKCLENRVAVLENQNKTLIEELKALKDLYCHKVE

SEQ ID NO: 182 - protein, 59 aa, synthetic construct
KEEATRKRELRLMKNREAARECRRKKKEYVKCLENRVAVLENQNKTLIEELKALRDLYC

SEQ ID NO: 183 - protein, 32 aa, synthetic construct
EYVKCLESRVAVLEVQNKKLIEELETLKDICS

SEQ ID NO: 184 - protein, 62 aa, synthetic construct
KEAARKREVRLMKNREAARECRRKKKEYVKCLENRVAVLENQNKTLIEELKALKDLYCHKSD

SEQ ID NO: 185 - protein, 68 aa, synthetic construct
EKRRLNDLVLENKLIALGEENATLKAELLSLKLKFGLISSTAYAQEIQKLSNSTAVYFQDYQTSKSNV

SEQ ID NO: 186 - protein, 58 aa, synthetic construct
QRKLERIARLEEKVKTLKAQNSELASTANMLREQVAQLKQKVMNHVNSGCQLMLTQQY

SEQ ID NO: 187 - protein, 66 aa, synthetic construct
QRVQQRHVLESEKNQLLQQVDHLKQEISRLVRERDAYKEKYEKLVSSGFRENGSSSDNPSSPEFFM

SEQ ID NO: 188 - protein, 68 aa, synthetic construct
QYKRVQQKHHLENEKTQLIQQVEQLKQEVSRLARERDAYKVKCEKLANSGFREAGSTSDSPSSPEFFL

SEQ ID NO: 189 - protein, 51 aa, synthetic construct
EYVECLEKKVETFTSENNELWKKVETLENANRTLLQQLQKLQTLVTNKISR

SEQ ID NO: 190 - protein, 50 aa, synthetic construct
EYIDGLETRMSACTAQNQELQRKVLHLEKQNLSLLEQLKKLQAIVVQSTS

SEQ ID NO: 191 - protein, 73 aa, synthetic construct
WRRKIRNKRSAQESRRKKKVYVGGLESRVLKYTAQNMELQNKVQLLEEQNLSLLDQLRKLQAMVIEISNKTSS

SEQ ID NO: 192 - protein, 78 aa, synthetic construct
WRKIRNKQSAQDSRRRKKEYIDGLESRVAACSAQNQELQKKVQELERHNISLVAQLRQLQTLIAQTSNKAAQTSTCVL

SEQ ID NO: 193 - protein, 75 aa, synthetic construct
EEQKDEKYWSRRYKNNEAAKRSRDARRLKENQISVRAAFLEKENALLRQEVVAVRQELSHYRAVLSRYQAQHGAL

SEQ ID NO: 194 - protein, 45 aa, synthetic construct
LKENQIAIRASFLEKENSALRQEVADLRKELGKCKNILAKYEARH

SEQ ID NO: 195 - protein, 68 aa, synthetic construct
NWTRRKKNNVAAKRSRDARRLKENQITIRAAFLEKENTALRTEVAELRKEVGKCKTIVSKYETKYGPL

SEQ ID NO: 196 - protein, 89 aa, synthetic construct
GVTQKEELEKQKAELQQEVEKLASENASMKLELDALRSKYEALQTFARTVARSPVAPARGPLAAGLGPLVPGKVAATSVITIVKSKTDA

SEQ ID NO: 197 - protein, 50 aa, synthetic construct
TQKEELERQRVELQQEVEKLARENSSMRLELDALRSKYEALQTFARTVAR

SEQ ID NO: 198 - protein, 71 aa, synthetic construct
QTARDRKKARMSELEQQVVDLEEENQKLLLENQLLREKTHGLVVENQELRQRLGMDALVAEEEAEAKGNEV

SEQ ID NO: 199 - protein, 55 aa, synthetic construct
PSQNAFRKRKLERLEELEKKEAQLTVTNDQIHILKKENELLHFMLRSLLTERNMP

SEQ ID NO: 200 - protein, 54 aa, synthetic construct
KRAAQNRSAQKAFRQRREKYIKNLEEKSKLFDGLMKENSELKKMIESLKSKLKE

SEQ ID NO: 201 - protein, 105 aa, synthetic construct
EDTPPNPKKEIEFPGGSPGRKRIDSEAKSRRTAQNRAAQRAFRDRKEAKMKSLQERVELLEQKDAQNKTTTDFLLCSLKSLLSEITKYRAKNSDDERILAFLDDL

SEQ ID NO: 202 - protein, 79 aa, synthetic construct
EDTPPNPKKEIEFQAKKKAQNRAAQKAFRERKEARMKELQDKLLESERNRQSLLKEIEELRKANTEINAENRLLLRSGN

SEQ ID NO: 203 - protein, 94 aa, synthetic construct
EDTPPNPKKEIEFPKRQNRDAQRAYRERKNNKLQVLEETIESLSKVVKNYETKLNRLQNELQAKESENHALKQKLETLTLKQASVPAQDPILQN

SEQ ID NO: 204 - protein, 81 aa, synthetic construct
QQNRDAQRAYRERRTTRIQVLEEKVEMLHNLVDDWQRKYKLLESEFSDTKENLQKSIALNNELQKALPLIVNTPFQQQPEN

SEQ ID NO: 205 - protein, 71 aa, synthetic construct
EDTPPNPKKEIEFHPAALKRARNTEAARRSRARKLQRMKQLEDKVEELLSKNYHLENEVARLKKLVGDAAR

SEQ ID NO: 206 - protein, 75 aa, synthetic construct
EDTPPNPKKEIEFDDDEDLLIQSKKSHQKKKLKEKELESSIHELTEIAASLQKRIHTLETENKLLKNLVLSSGET

SEQ ID NO: 207 - protein, 20 aa, synthetic construct
MSYYHHHHHHLESTSLYKKA

SEQ ID NO: 208 - protein, 4 aa, synthetic construct
GSGS

SEQ ID NO: 209 - protein, 4 aa, synthetic construct
GSEF

SEQ ID NO: 210 - protein, 4 aa, synthetic construct
GSGR

SEQ ID NO: 211 - protein, 4 aa, synthetic construct
GFDD

SEQ ID NO: 212 - protein, 5 aa, synthetic construct
YLEII

SEQ ID NO: 213 - protein, 17 aa, synthetic construct
CSSNTQLSCTKWLIRGC

SEQ ID NO: 214 - protein, 16 aa, synthetic construct
SSNTQLSCTKWLIRGC

SEQ ID NO: 215 - protein, 6 aa, synthetic construct
STCRDL

SEQ ID NO: 216 - protein, 7 aa, synthetic construct
FSTCRDL

SEQ ID NO: 217 - protein, 11 aa, synthetic construct
LDLQRSMNRRY

SEQ ID NO: 218 - protein, 7 aa, synthetic construct
PAEIYES

SEQ ID NO: 219 - DNA, 513 bp, synthetic construct
  source 1..513: mol_type = other DNA
gcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

SEQ ID NO: 220 - protein, 171 aa, synthetic construct
AAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 221 - protein, 611 aa, synthetic construct
MDWIWRILFLVGAATGAHSDYKDDDDKIARLEEKVKTLKAQNSELASTANMLREQVAQLKQKVMNHFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMPAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 222 - DNA, 1833 bp, synthetic construct
  source 1..1833: mol_type = other DNA
atggattggatctggcggatcctgttccttgtgggagctgccacaggcgcccacagcgactacaaggacgacgacgacaagatcgccaggctggaggagaaggtgaagaccctgaaggcccagaacagcgagctggccagcaccgccaacatgctgagggagcaggtggcccagctgaagcagaaggtgatgaaccacttcccctggctgctgatcatcatcttcggcatctttggcctgaccgtgatgctgttcgtgttcctgttcagcaagcagcagcggatcaagatgctgatcctgcctcctgtgcctgtgcctaagatcaagggcatcgaccccgacctgctgaaagagggcaagctggaagaagtgaacaccatcctggccatccacgacagctacaagcccgagttccacagcgacgatagctgggtcgagttcatcgagctggacatcgacgagcccgacgagaaaaccgaggaaagcgacaccgacagactgctgagcagcgaccacgagaagtcccactctaacctgggcgtgaaggatggcgatagcggcagaacaagctgctgcgagcccgatatcctggaaaccgacttcaacgccaacgacatccacgagggcaccagcgaagttgcccagcctcaaagactgaagggcgaagccgatctgctgtgcctggaccagaagaaccagaacaacagcccctaccacgacgcctgtcctgctacacagcagcctagtgtgatccaggccgagaagaacaagccccagcctctgcctacagagggcgccgaatctacacatcaggccgctcacatccagctgagcaaccctagcagcctgagcaacatcgacttctacgcccaagtgtccgacatcacaccagccggatctgtggtgctgtctcccggccagaaaaacaaggccggcatgtcccagtgcgacatgcaccctgaaatggtgtccctgtgccaagagaacttcctgatggacaacgcctacttctgcgaggccgacgccaagaaatgcatccctgtggctccccacatcaaggtggaaagccacattcagcccagcctgaatcaagaggatatctacatcaccaccgagagcctgaccaccgctgctggtagacctggaacaggcgagcatgtgcctggctctgaaatgcccgtgcctgactacaccagcatccacatcgtgcagagccctcagggcctgatcctgaatgctacagctctgcccctgccagacaaagagttcctgtctagctgcggctacgtgtccaccgaccagctgaacaagatcatgcccgcggctggagggccaggagctggctctgccgcgccggtgtcttccaccagttcactgcctttggcggctcttaacatgcgggtcaggcgaagattgagtttgtttctgaatgttaggactcaagtagctgccgactggacggcccttgcggaagaaatggacttcgagtatctagagattcgccaacttgaaacccaggcagatcctacaggcagactcctcgatgcgtggcagggtcggcctggagcatctgtgggaaggttgctggaactgttgactaaacttggtcgagatgacgtactgctggaattgggtccaagcatcgaagaagattgtcaaaaatacatcctcaagcagcaacaagaggaagcagagaaaccattgcaagtagcggcagtagactctagcgtaccgcgcactgctgagcttgctggaatcacgacccttgacgatcctctcgggcatatgccagagcggttcgacgcattcatatgttattgtccaagcgatatc

(19) United States
(12) Patent Application Publication
(10) Pub. No.: US 2026/0306631 A1
(43) Pub. Date: Oct. 8, 2026

(54) CHIMERIC RECEPTORS

(71) Applicant: St. Jude Children's Research Hospital, Inc., Memphis, TN (US)

(72) Inventors: Stephen GOTTSCHALK, Memphis, TN (US); Matthew BELL, Memphis, TN (US); Shannon LANGE, Memphis, TN (US)

(73) Assignee: St. Jude Children's Research Hospital, Inc., Memphis, TN (US)

(21) Appl. No.: 19/476,468

(22) PCT Filed: Apr. 19, 2024
(86) PCT No.: PCT/US2024/025498

§ 371 Date: Oct. 17, 2025

Related U.S. Application Data
(60) Provisional application No. 63/497,140, filed on Apr. 19, 2023.

(51) Int. Cl. A61K 40/31; A61K 40/11; A61P 35/00; C07K 14/47; C07K 14/705; C07K 14/71; C07K 14/715; C07K 14/72; C12N 5/0783; C12N 9/90

(52) U.S. Cl. CPC A61K 40/31; A61K 40/11; A61P 35/00; C07K 14/47; C07K 14/70521; C07K 14/71; C07K 14/7155; C07K 14/72; C12N 5/0636; C12N 9/90; C12Y 502/01008; C07K 2319/02; C07K 2319/03; C07K 2319/41; C07K 2319/43; C12N 2510/00

(57) ABSTRACT

The application relates to chimeric receptors, particularly chimeric receptors comprising homodimeric polypeptides. Also disclosed are the uses of these receptors in tumor immunotherapy adoptive cell therapy). The application further relates to methods of genetically modifying therapeutic immune cells resulting in an enhanced immune response against a target antigen. Them application further relates to therapeutic cells that express the chimeric receptors and methods for treating patients using the modified therapeutic cells.

### CROSS-REFERENCE TO RELATED APPLICATIONS

This application claims priority to U.S. Provisional Application No. 63/497,140, filed Apr. 19, 2023, the disclosure of which is herein incorporated by reference in its entirety.

### STATEMENT REGARDING FEDERALLY SPONSORED RESEARCH OR DEVELOPMENT

This invention was made with government support under CA250401 awarded by National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Apr. 19, 2024, is named 243734_000199_SL.xml and is 235,430 bytes in size.

### FIELD OF THE INVENTION

The application relates to chimeric receptors, particularly chimeric receptors comprising homodimeric polypeptides which comprise at least one homodimerizing motif. The application also relates to the uses of these chimeric receptors in tumor immunotherapy (e.g., adoptive cell therapy). The application further relates to methods of genetically modifying therapeutic immune cells resulting in an enhanced immune response against a target antigen. The application further relates to therapeutic cells that express the chimeric receptors and methods for treating patients using the modified therapeutic cells.

### BACKGROUND

Immunotherapy with immune cells including T cells expressing chimeric antigen receptors (CARs) has already revolutionized the treatment approach for patients with hematological malignancies. However, immune cells, including CAR T cells, have been less effective for solid tumors and brain tumors. While lack of efficacy is most likely multifactorial, limited persistence without stimulation and an inability to function in the setting of chronic antigen exposure have emerged as major roadblocks.

To improve the ability of CAR T cells to remain viable without antigen exposure and function in the setting of chronic antigen exposure, gamma cytokines, including IL-2, IL-7, IL-15, and IL-21, or members of other cytokine families (e.g., IL-18) have been overexpressed in CAR T cells. Cytokine-expressing CAR T cells have improved antitumor activity in preclinical models, but there are safety concerns since these secreted or membrane-bound cytokines have the potential to activate bystander immune cells. Accordingly, there is a need to develop chimeric receptors that can provide signals to the genetically modified immune cells.

### SUMMARY OF THE INVENTION

Disclosed herein are chimeric receptors, particularly chimeric receptors comprising homodimeric polypeptides which comprise at least one homodimerizing motif. Also disclosed are the uses of the chimeric receptors in tumor immunotherapy (e.g., adoptive cell therapy). Additionally disclosed are methods of genetically modifying therapeutic immune cells resulting in an enhanced immune response against a target antigen. Therapeutic cells that express the chimeric receptors and methods for treating patients using the modified therapeutic cells are also disclosed.

In one aspect, provided herein is a chimeric receptor which is a homodimer comprising two polypeptide monomers, wherein each polypeptide monomer comprises:
i) an extracellular region comprising at least one homodimerizing motif;
ii) a transmembrane region; and
iii) an intracellular region comprising at least one signaling region derived from a cell-surface receptor or a signal transducing adaptor protein, or a portion or variant thereof.

In some embodiments, the extracellular region comprises at least one homodimerizing leucine zipper motif, at least one ligand-dependent homodimerizing motif, or at least one single chain variable fragment, or combinations thereof.

In some embodiments, the extracellular region comprises at least one homodimerizing leucine zipper motif. In some embodiments, the homodimerizing leucine zipper motif comprises at least five heptad repeats of amino acids with a leucine at every seventh position. In some embodiments, the homodimerizing leucine zipper motif is derived from transcription factor c-Jun. In some embodiments, the homodimerizing leucine zipper motif comprises the amino acid sequence of SEQ ID NO: 9, or a sequence having at least 80% identity thereto. In some embodiments, the homodimerizing leucine zipper motif is encoded by the nucleotide sequence of SEQ ID NO: 3, or a sequence having at least 80% identity thereto.

In some embodiments, the ligand-dependent homodimerizing motif undergoes homodimerization in the presence of a chemical inducer. In some embodiments, the ligand-dependent homodimerizing motif comprises an FKBP12_{F36V} domain.

In some embodiments, the signaling region derived from a cell-surface receptor or a signal transducing adaptor protein, or a portion or variant thereof, activates Janus kinase (JAK)-signal transducer and activator of transcription (STAT) pathway, nuclear factor kappa-light-chain-enhancer of activated B cells (NFκB) pathway, and/or additional signaling pathways activated by the MyD88 signaling complex (Myddosome).

In some embodiments, the additional signaling pathways activated by the MyD88 signaling complex (Myddosome) are one or more of interleukin-1 receptor-associated kinase (IRAK), TNF receptor (TNFR)-associated factor (TRAF), TANK-binding kinase (TBK), mitogen-activated protein kinase (MAPK), protein kinase B or AKT (PKB/AKT), and/or phosphatidylinositol-3-kinase (PI3K) pathways.

In some embodiments, the intracellular region comprises a first signaling region derived from a cell-surface receptor, or a portion or variant thereof, and a second signaling region derived from a signal transducing adaptor protein, or a portion or variant thereof.

In some embodiments, the signaling region is derived from a cytokine receptor, or a portion or variant thereof. In some embodiments, the cytokine receptor, or a portion or variant thereof, is erythropoietin receptor (EpoR), growth hormone receptor (GHR), prolactin receptor (PRLR), leptin receptor (LEPR), granulocyte colony stimulating factor (G-CSFR), thrombopoietin receptor (TpoR), interleukin-23 receptor (IL-23R), interleukin 10 receptor beta subunit (IL-10R2), IL-6 beta chain (gp130), IL-2Rβ, IL-18Rα, or IL-18Rβ.

In some embodiments, the signaling region is derived from EpoR, or a portion or variant thereof. In some embodiments, the signaling region derived from EpoR does not comprise a Src homology region 2 domain-containing phosphatase-1 (SHP1) binding site. In some embodiments, the signaling region derived from EpoR comprises the amino acid sequence of SEQ ID NO: 46, or a sequence having at least 80% identity thereto. In some embodiments, the signaling region derived from EpoR is encoded by the nucleotide sequence of SEQ ID NO: 45, or a sequence having at least 80% identity thereto. In some embodiments, the signaling region derived from EpoR comprises the amino acid sequence of SEQ ID NO: 49, or a sequence having at least 80% identity thereto. In some embodiments, the signaling region derived from EpoR is encoded by the nucleotide sequence of SEQ ID NO: 154, or a sequence having at least 80% identity thereto.

In some embodiments, the signaling region is derived from GHR, or a portion or variant thereof. In some embodiments, the signaling region derived from GHR comprises the amino acid sequence of SEQ ID NO: 44, or a sequence having at least 80% identity thereto. In some embodiments, the signaling region derived from GHR is encoded by the nucleotide sequence of SEQ ID NO: 43, or a sequence having at least 80% identity thereto.

In some embodiments, the signaling region is derived from a receptor tyrosine kinase, or a portion or a variant thereof. In some embodiments, the receptor tyrosine kinase is epidermal growth factor receptor (EGFR).

In some embodiments, the signaling region is derived from a Toll-like receptor, or a portion or a variant thereof. In some embodiments, the Toll-like receptor is TLR1, TLR2, TLR3, TLR4, TLR5, TLR6, TLR7, TLR8, TLR9, or TLR10.

In some embodiments, the signaling region is derived from a signal transducing adaptor protein, or a portion or a variant thereof. In some embodiments, the signal transducing adaptor protein is MyD88. In some embodiments, the signaling region derived from MyD88 comprises the amino acid sequence of SEQ ID NO: 38 or 220, or a sequence having at least 80% identity thereto. In some embodiments, the signaling region derived from MyD88 is encoded by the nucleotide sequence of SEQ ID NO: 37 or 219, or a sequence having at least 80% identity thereto.

In some embodiments, the transmembrane region is derived from the same cell-surface receptor as the signaling region. In some embodiments, the transmembrane region is derived from EpoR, or a portion or variant thereof. In some embodiments, the transmembrane region derived from EpoR comprises the amino acid sequence of SEQ ID NO: 48, SEQ ID NO: 104, SEQ ID NO: 128, SEQ ID NO: 106, SEQ ID NO: 130, SEQ ID NO: 108, or SEQ ID NO: 132, or a sequence having at least 80% identity thereto. In some embodiments, the transmembrane region derived from EpoR is encoded by the nucleotide sequence of SEQ ID NO: 103, SEQ ID NO: 105, SEQ ID NO: 127, SEQ ID NO: 107, SEQ ID NO: 129, SEQ ID NO: 109 or SEQ ID NO: 131, or a sequence having at least 80% identity thereto.

In some embodiments, the transmembrane region is derived from GHR, or a portion or variant thereof. In some embodiments, the transmembrane region derived from GHR comprises the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a sequence having at least 80% identity thereto. In some embodiments, the transmembrane region derived from GHR is encoded by the nucleotide sequence of SEQ ID NO: 133, SEQ ID NO: 135, SEQ ID NO: 137, SEQ ID NO: 139, SEQ ID NO: 141, SEQ ID NO: 143, or SEQ ID NO: 145, or a sequence having at least 80% identity thereto.

In some embodiments, the transmembrane regions is derived from IL-18Ra, IL-18RB, CD28, IL-2RB, CD8, CD4, CD35, CD40, CD134 (OX-40), CD19, or CD7. In some embodiments, the transmembrane region is derived from IL-18Ra, or a portion or variant thereof.

In some embodiments, the transmembrane region derived from IL-18Ra comprises the amino acid sequence of SEQ ID NO: 40, or a sequence having at least 80% identity thereto. In some embodiments, the transmembrane region derived from IL-18Ra is encoded by the nucleotide sequence of SEQ ID NO: 39, or a sequence having at least 80% identity thereto.

In some embodiments, the transmembrane region is derived from IL-18RB, or a portion or variant thereof. In some embodiments, the transmembrane region derived from IL-18RB comprises the amino acid sequence of SEQ ID NO: 36, or a sequence having at least 80% identity thereto. In some embodiments, the transmembrane region derived from IL-18RB is encoded by the nucleotide sequence of SEQ ID NO: 35, or a sequence having at least 80% identity thereto.

In some embodiments, the transmembrane region is derived from CD28, or a portion or variant thereof. In some embodiments, the transmembrane region derived from CD28 comprises the amino acid sequence of SEQ ID NO: 42, or a sequence having at least 80% identity thereto. In some embodiments, the transmembrane region derived from CD28 is encoded by the nucleotide sequence of SEQ ID NO: 41, or a sequence having at least 80% identity thereto.

In some embodiments, each polypeptide monomer of the chimeric receptor comprises:
i) an extracellular region comprising a homodimerizing leucine zipper motif;
ii) a transmembrane region derived from EpoR, or a portion or variant thereof; and
iii) an intracellular region comprising a signaling region derived from EpoR, or a portion or variant thereof.

In some embodiments, the signaling region derived from EpoR does not comprise the Src homology region 2 domain-containing phosphatase-1 (SHP1) binding site. In some embodiments, the signaling region derived from EpoR comprises the amino acid sequence of SEQ ID NO: 46, or a sequence having at least 80% identity thereto. In some embodiments, the signaling region derived from EpoR is encoded by the nucleotide sequence of SEQ ID NO: 45, or a sequence having at least 80% identity thereto. In some embodiments, the transmembrane region derived from EpoR comprises the amino acid sequence of SEQ ID NO: 48, SEQ ID NO: 104, SEQ ID NO: 128, SEQ ID NO: 106, SEQ ID NO: 130, SEQ ID NO: 108, or SEQ ID NO: 132, or a sequence having at least 80% identity thereto. In some embodiments, the transmembrane region derived from EpoR is encoded by the nucleotide sequence of SEQ ID NO: 103, SEQ ID NO: 105, SEQ ID NO: 127, SEQ ID NO: 107, SEQ ID NO: 129, SEQ ID NO: 109, or SEQ ID NO: 131, or a sequence having at least 80% identity thereto. In some embodiments, each polypeptide monomer comprises the amino acid sequence of SEQ ID NO: 112, 114, 116, or 118, or a sequence having at least 80% identity thereto. In some embodiments, each polypeptide monomer is encoded by the nucleotide sequence of SEQ ID NO: 111, 113, 115, or 117, or a sequence having at least 80% identity thereto.

In some embodiments, each polypeptide monomer of the chimeric receptor comprises:
i) an extracellular region comprising a homodimerizing leucine zipper motif;
ii) a transmembrane region derived from GHR, or a portion or variant thereof; and
iii) an intracellular region comprising a signaling region derived from GHR, or a portion or variant thereof.

In some embodiments, the signaling region derived from GHR comprises the amino acid sequence of SEQ ID NO: 44, or a sequence having at least 80% identity thereto. In some embodiments, the signaling region derived from GHR is encoded by the nucleotide sequence of SEQ ID NO: 43, or a sequence having at least 80% identity thereto. In some embodiments, the transmembrane region derived from GHR comprises the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a sequence having at least 80% identity thereto. In some embodiments, the transmembrane region derived from GHR is encoded by the nucleotide sequence of SEQ ID NO: 133, SEQ ID NO: 135, SEQ ID NO: 137, SEQ ID NO: 139, SEQ ID NO: 141, SEQ ID NO: 143, or SEQ ID NO: 145. In some embodiments, each polypeptide monomer comprises the amino acid sequence of SEQ ID NO: 120, or a sequence having at least 80% identity thereto. In some embodiments, each polypeptide is encoded by the nucleotide sequence of SEQ ID NO: 119, or a sequence having at least 80% identity thereto.

In some embodiments, each polypeptide monomer of the chimeric receptor comprises:
i) an extracellular region comprising a homodimerizing leucine zipper motif;
ii) a transmembrane region derived from IL-18Ra, IL-18RB or CD28, or a portion or variant thereof; and
iii) an intracellular region comprising a signaling region derived from MyD88, or a portion or variant thereof.

In some embodiments, the signaling region derived from MyD88 comprises the amino acid sequence of SEQ ID NO: 38 or 220, or a sequence having at least 80% identity thereto. In some embodiments, the signaling region derived from MyD88 is encoded by the nucleotide sequence of SEQ ID NO: 37 or 219, or a sequence having at least 80% identity thereto. In some embodiments, the transmembrane region comprises the amino acid sequence of SEQ ID NO: 40, SEQ ID NO: 36, or SEQ ID NO: 42, or a sequence having at least 80% identity thereto. In some embodiments, the transmembrane region is encoded by the nucleotide sequence of SEQ ID NO: 39, SEQ ID NO: 35, or SEQ ID NO: 41, or a sequence having at least 80% identity thereto. In some embodiments, each polypeptide monomer comprises the amino acid sequence of SEQ ID NO: 122, 124, or 126, or a sequence having at least 80% identity thereto. In some embodiments, each polypeptide monomer is encoded by the nucleotide sequence of SEQ ID NO: 121, 123, or 125, or a sequence having at least 80% identity thereto.

In some embodiments, each polypeptide monomer of the chimeric receptor comprises:
i) an extracellular region comprising a homodimerizing leucine zipper motif;
ii) a transmembrane region derived from GHR, or a portion or variant thereof; and
iii) an intracellular region comprising a signaling region derived from GHR, or a portion or variant thereof, and a signaling region derived from MyD88, or a portion or variant thereof.

In some embodiments, the signaling region derived from GHR comprises the amino acid sequence of SEQ ID NO: 44, or a sequence having at least 80% identity thereto. In some embodiments, the signaling region derived from GHR is encoded by the nucleotide sequence of SEQ ID NO: 43, or a sequence having at least 80% identity thereto. In some embodiments, the signaling region derived from MyD88 comprises the amino acid sequence of SEQ ID NO: 38 or SEQ ID NO: 220, or a sequence having at least 80% identity thereto. In some embodiments, the signaling region derived from MyD88 is encoded by the nucleotide sequence of SEQ ID NO: 37 or SEQ ID NO: 219, or a sequence having at least 80% identity thereto. In some embodiments, the transmembrane region derived from GHR comprises the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a sequence having at least 80% identity thereto. In some embodiments, the transmembrane region derived from GHR is encoded by the nucleotide sequence of SEQ ID NO: 133, SEQ ID NO: 135, SEQ ID NO: 137, SEQ ID NO: 139, SEQ ID NO: 141, SEQ ID NO: 143, or SEQ ID NO: 145. In some embodiments, each polypeptide monomer comprises amino acids 28-611 of the amino acid sequence of SEQ ID NO: 221, or a sequence having at least 80% identity thereto. In some embodiments, each polypeptide monomer is encoded by nucleotides 82-1833 of the nucleotide sequence of SEQ ID NO: 222, or a sequence having at least 80% identity thereto.

In some embodiments, each polypeptide monomer of the chimeric receptor further comprises a leader sequence. In some embodiments, the leader sequence is derived from an immunoglobulin heavy chain variable region or colony stimulating factor 2 receptor alpha chain (CSF2RA). In some embodiments, the leader sequence derived from an immunoglobulin heavy chain variable region comprises the amino acid sequence SEQ ID NO: 7.

In some embodiments, each polypeptide monomer further comprises a hinge region. In some embodiments, the hinge region is derived from the same cell-surface receptor as the signaling region. In some embodiments, the hinge regions is derived from IgG1, IgG2, IgG3, IgG4, CD28, or CD8α.

In some embodiments, the polypeptide monomer further comprises one or more additional polypeptide sequences. In some embodiments, the one or more additional polypeptide sequences comprise are selected from one or more cellular markers, epitope tags, cytokines, safety switches, dimerization moieties, and degradation moieties.

In another aspect, provided herein is a polynucleotide encoding the chimeric receptor described herein. In some embodiments, the nucleotide sequence(s) is expressed in an inducible fashion, achieved with an inducible promoter, an inducible expression system, an artificial signaling circuit, and/or drug induced splicing. In some embodiments, the nucleotide sequence is operably linked to a promoter. In some embodiments, the promoter is an inducible promoter. In some embodiments, the promoter is a T cell-specific promoter or an NK cell-specific promoter.

In some embodiments, the polynucleotide further comprises one or more additional nucleotide sequences encoding one or more additional polypeptide sequences. In some embodiments, the one or more additional polypeptide sequences are selected from one or more cellular markers, epitope tags, cytokines, safety switches, dimerization moieties, and degradation moieties. In some embodiments, the epitope tag is FLAG or Myc. In some embodiments, the cellular marker is mClover3 or mRuby. In some embodiments, the nucleotide sequence encoding the chimeric receptor is operably linked to the one or more additional nucleotide sequences encoding one or more additional polypeptide sequences via a sequence encoding a self-cleaving peptide and/or an internal ribosomal entry site (IRES). In some embodiments, the self-cleaving peptide is a 2A peptide. In some embodiments, the 2A peptide is T2A, P2A, E2A, or F2A peptide. In some embodiments, the 2A peptide is a P2A peptide. In some embodiments, the P2A peptide comprises the amino acid sequence SEQ ID NO: 11, or an amino acid sequence having at least 80% sequence identity thereof.

In some embodiments, the polynucleotide described herein is a DNA molecule. In some embodiments, the polynucleotide described herein is an RNA molecule.

In another aspect, provided herein is a recombinant vector comprising the polynucleotide described herein. In some embodiments, the vector is a viral vector. In some embodiments, the viral vector is a retroviral vector, a lentiviral vector, an adenoviral vector, an adeno-associated virus vector, an alphaviral vector, a herpes virus vector, a baculoviral vector, or a vaccinia virus vector. In some embodiments, the viral vector is a retroviral vector. In some embodiments, the vector is a non-viral vector. In some embodiments, the non-viral vector is a minicircle plasmid, a Sleeping Beauty transposon, a piggyBac transposon, or a single or double stranded DNA molecule that is used as a template for homology directed repair (HDR) based gene editing.

In another aspect, provided herein is an isolated host cell comprising the polynucleotide described herein or the recombinant vector described herein. In another aspect, provided herein is an isolated host cell comprising a chimeric receptor described herein.

In some embodiments, the host cell is an immune cell. In some embodiments, the host cell is a T cell, a natural killer (NK) cell, a mesenchymal stem cell (MSC), or a macrophage. In some embodiments, the host cell is a T cell. In some embodiments, the host cell is an αβ T cell receptor (TCR) T cell, a γδ T cell, a CD8+ T cell, a CD4+ T cell, a cytotoxic T cell, an invariant natural killer T (iNKT) cell, a memory T cell, a memory stem T cell (T_{SCM}), a naïve T cell, an effector T cell, a T-helper cell, or a regulatory T cell (Treg). In some embodiments, the host cell is a NK cell. In some embodiments, the host cell is a NK cell derived from peripheral, cord blood, induced pluripotent stem (iPS) cells (iPSCs), and/or a cell line (e.g., NK92 cells).

In some embodiments, the host cell further expresses one or more antigen-recognition molecules. In some embodiments, the one or more antigen-recognition molecules are selected from chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

In some embodiments, the host cell is further genetically modified to enhance its function by expressing one or more additional genes or deleting one or more inhibitory genes with a gene editing technology (e.g., CRISPR-Cas9 or transcription activator-like effector nuclease (TALEN)). In some embodiments, the one or more additional genes are selected from one or more transcription factors. In some embodiments, the transcription factor is c-Jun. In some embodiments, the one or more inhibitory genes is REGNASE-1 or DNMT3A. In some embodiments, the gene editing technology is CRISPR-Cas9 or transcription activator-like effector nuclease (TALEN).

In some embodiments, the host cell has been activated and/or expanded ex vivo.

In some embodiments, the host cell is an allogeneic cell. In some embodiments, the host cell is an autologous cell.

In some embodiments, the immune cell is derived from an induced pluripotent stem (iPS) cell (iPSC).

In another aspect, provided herein is a pharmaceutical composition comprising the host cell described herein and a pharmaceutically acceptable carrier and/or excipient.

In another aspect, provided herein is a method of enhancing an effector function of an immune cell, wherein the immune cell expresses one or more antigen-recognition molecules comprising genetically modifying the cell with the polynucleotide described herein or the recombinant vector described herein. In some embodiments, the one or more antigen-recognition molecules are selected from chimeric antigen receptors (CARs), T cell receptor fusion constructs (TruCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof. In some embodiments, the effector function is one or more of expansion, persistence, and/or cytotoxicity (e.g., anti-tumor activity).

In another aspect, provided herein is a method of generating the isolated host cell described herein, said method comprising genetically modifying the host cell with the polynucleotide of described herein or the recombinant vector described herein. In some embodiments, the method further comprises genetically modifying the host cell to express one or more antigen-recognition molecules. In some embodiments, the one or more antigen-recognition molecules are selected from chimeric antigen receptors (CARs), T cell receptor fusion constructs (TruCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof. In some embodiments, the genetic modifying step is conducted via viral gene delivery. In some embodiments, the genetic modifying step is conducted via non-viral gene delivery. In some embodiments, the genetically modifying step is conducted ex vivo. In some embodiments, the method further comprises activation and/or expansion of the host cell ex vivo before, after, and/or during said genetic modification.

In another aspect, provided herein is a method of treating a disease comprising administering to a subject an effective amount of the host cell described herein, or the pharmaceutical composition described herein. In some embodiments, the method comprises:
a) isolating T cells or NK cells from the subject or donor;
b) modifying the T cells or NK cells ex vivo with a polynucleotide described herein or a recombinant vector described herein;
c) optionally, modifying the T cells or NK cells ex vivo to express one or more antigen-recognition molecules that bind an antigen associated with the disease;
d) optionally, expanding and/or activating the modified T cells or NK cells before, after, and/or during step b) or c); and
e) introducing a therapeutically effective amount of the modified T cells or NK cells into the subject.

In some embodiments, the one or more antigen-recognition molecules are selected from chimeric antigen receptors (CARs), T cell receptor fusion constructs (TruCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

In some embodiments, the disease is a cancer, infection, or autoimmune disease.

In some embodiments, the subject is human.

In another aspect, provided herein is a chimeric receptor which is a homodimer comprising two polypeptide monomers, wherein each polypeptide monomer comprises:
i) an extracellular region comprising a means for homodimerizing the two polypeptide monomers;
ii) a transmembrane region comprising a means for joining the intracellular regions with extracellular region; and
iii) an intracellular region comprising a means for enhancing effector function of an immune cell.

In some embodiments, the means for homodimerizing the two polypeptide monomers comprises at least one homodimerizing leucine zipper motif, at least one ligand-dependent homodimerizing motif, or at least one single chain variable fragment, or combinations thereof. In some embodiments, the means for homodimerizing the two poly-peptide monomers comprises at least one homodimerizing leucine zipper motif.

In some embodiments, the homodimerizing leucine zipper motif comprises at least five heptad repeats of amino acids with a leucine at every seventh position. In some embodiments, the homodimerizing leucine zipper motif is derived from transcription factor c-Jun. In some embodiments, the homodimerizing leucine zipper motif comprises the amino acid sequence of SEQ ID NO: 9, or a sequence having at least 80% identity thereto. In some embodiments, the homodimerizing leucine zipper motif is encoded by the nucleotide sequence of SEQ ID NO: 3, or a sequence having at least 80% identity thereto.

In some embodiments, the ligand-dependent homodimerizing motif undergoes homodimerization in the presence of a chemical inducer.

In some embodiments, the means for enhancing effector function comprises at least one signaling region derived from a cell-surface receptor or a signal transducing adaptor protein, or a portion or variant thereof. In some embodiments, the means for enhancing effector function comprises a signaling region derived from EpoR, or a portion or variant thereof. In some embodiments, the means for enhancing effector function comprises a signaling region derived from GHR, or a portion or variant thereof. In some embodiments, the means for enhancing effector function comprises a signaling region derived from MyD88, or a portion or variant thereof. In some embodiments, the means for enhancing effector function comprises a signaling region derived from GHR, or a portion or variant thereof, and a signaling region derived from MyD88, or a portion or variant thereof.

In another aspect, provided herein is a chimeric receptor which is a homodimer comprising two polypeptide monomers, wherein each polypeptide monomer comprises:
i) an extracellular region comprising a means for homodimerizing the two polypeptide monomers;
ii) a transmembrane region; and
iii) an intracellular region comprising at least one signaling region derived from a cell-surface receptor or a signal transducing adaptor protein, or a portion or variant thereof.

In another aspect, provided herein is a chimeric receptor which is a homodimer comprising two polypeptide monomers, wherein each polypeptide monomer comprises:
i) an extracellular region comprising at least one homodimerizing motif;
ii) a transmembrane region; and
iii) an intracellular region comprising a means for enhancing effector function of an immune cell.

In another aspect, provided herein is a chimeric receptor comprising i) an extracellular region; ii) a transmembrane region; and iii) an intracellular region comprising at least one signaling region derived from a cell-surface receptor or a signal transducing adaptor protein, or a portion or variant thereof, the improvement comprising said extracellular region comprising at least one homodimerizing motif.

In another aspect, provided herein is a chimeric receptor, the improvement comprising two transmembrane polypeptide monomers each comprising i) an extracellular region comprising at least one homodimerizing motif and ii) an intracellular region comprising at least one signaling region derived from a cell-surface receptor or a signal transducing adaptor protein, or a portion or variant thereof.

In some embodiments, the improvement comprises said extracellular region comprising at least one homodimerizing leucine zipper motif, at least one ligand-dependent homodimerizing motif, or at least one single chain variable fragment, or combinations thereof. In some embodiments, the improvement comprises said extracellular region comprising at least one homodimerizing leucine zipper motif. In some embodiments, the improvement comprises said homodimerizing leucine zipper motif comprising at least five heptad repeats of amino acids with a leucine at every seventh position. In some embodiments, the improvement comprises said homodimerizing leucine zipper motif being derived from transcription factor c-Jun. In some embodiments, the improvement comprises said homodimerizing leucine zipper motif comprising the amino acid sequence of SEQ ID NO: 9, or a sequence having at least 80% identity thereto. In some embodiments, the improvement comprises said homodimerizing leucine zipper motif being encoded by the nucleotide sequence of SEQ ID NO: 3, or a sequence having at least 80% identity thereto. In some embodiments, the improvement comprises said ligand-dependent homodimerizing motif undergoing homodimerization in the presence of a chemical inducer. In some embodiments, the improvement further comprises said intracellular region comprising a signaling region derived from EpoR, or a portion or variant thereof. In some embodiments, the improvement further comprises said intracellular region comprising a signaling region derived from GHR, or a portion or variant thereof. In some embodiments, the improvement further comprises said intracellular region comprising a signaling region derived from MyD88, or a portion or variant thereof. In some embodiments, the improvement further comprises said intracellular region comprising a signaling region derived from GHR, or a portion or variant thereof, and a signaling region derived from MyD88, or a portion or variant thereof.

In another aspect, provided herein is a polynucleotide encoding a chimeric receptor described herein.

In another aspect, provided herein is a recombinant vector comprising a polynucleotide described herein.

In another aspect, provided herein is an immune cell expressing a chimeric receptor described herein.

In another aspect, provided herein is an immune cell comprising a polynucleotide described herein or a recombinant vector described herein.

In another aspect, provided herein is a method of enhancing an effector function of an immune cell, wherein the improvement comprises the immune cell expressing a chimeric receptor described herein.

In another aspect, provided herein is a method of improving expansion or persistence or cytotoxicity of an antigen-activated immune cell, wherein the improvement comprises the immune cell expressing a chimeric receptor described herein.

In another aspect, provided herein is a method of treating a disease comprising administering to a subject an effective amount of an immune cell, wherein the improvement comprises the immune cell expressing the chimeric receptor described herein.

### BRIEF DESCRIPTION OF THE DRAWINGS

FIGS. 1A-1D display Jun.MyD88 homodimers activating NFκB signaling and improving CAR T cell antitumor activity. (FIG. 1A) NFκB activity in Ramos Blue NFκB reporter cells transduced with indicated constructs (N=3 technical replicates, paired ANOVA, *p<0.05). (FIG. 1B) NFκB activity in Ramos Blue NFκB reporter cells transduced with indicated constructs (N=5 technical replicates, paired ANOVA, **** p<0.0001). (FIG. 1C) Transduction efficiency in human T cells (N=1). (FIG. 1D) Repeat stimulation assay with A673 tumor cells and EphA2-CAR T cells (N=1).

FIGS. 2A-2F show Jun leucine zipper-based homodimers activating STAT5 resulting in improved T cell viability, and that they are inhibited by ruxolitinib. (FIGS. 2A and 2B) pSTAT5 geometric mean fluorescence intensity (FIG. 2A) or percent positive (FIG. 2B) in Jun ZipR+ cells (N=4 biological replicates, One-way ANOVA, ** p<0.01, *** p<0.001, **** p<0.0001). (FIGS. 2C-2E) Frequency of pSTAT5+ cells following 48-hour treatment with indicated concentrations of ruxolitinib in vitro (N=3 biological replicates). (FIG. 2F) Frequency of dead cells (Annexin V+ Dead+), apoptotic cells (Annexin V+ Dead-), necrotic cells (Annexin V− Dead+), and live cells (Annexin V− Dead-) after 7 days of cytokine starvation without or with treatment of 5 M ruxolitinib in vitro (N=2 biological replicates, two-way ANOVA, **** p<0.0001 on Live cell population)

FIGS. 3A-3C show that Jun homodimeric ZipRs do not alter human T cell immunophenotype. (FIG. 3A) Transduction efficiency in human T cells (N=4, biological replicates). (FIG. 3B) Frequency of CD4+ or CD8+ cells (N=4, biological replicates). (FIG. 3C) T cell immunophenotype in CD4+ (left) or CD8+ (right) cells (N=4, biological replicates, TEMRA: Effector memory re-expressing CD45RA [CD45+CCR7-]; T_{EM}: Effector Memory [CD45RA−CCR7-], T_{CM}: Central Memory [CD45RA−CCR7+]; T_{N}-Like: Naïve-Like [CD45RA+CCR7+]).

FIGS. 4A-4B show successful transduction of a reporter cell line with retroviral constructs encoding Jun.GHR and Jun.GHR.MyD88 and corresponding phosphorylation of STAT5. FIG. 4A demonstrates the STAT5 reporter cell line was successfully transduced with retroviral constructs encoding Jun.GHR and Jun.GHR.MyD88. FIG. 4B demonstrates that Jun.GHR and Jun. GHR.MyD88 phosphorylate STAT5.

FIGS. 5A-5B show evaluation of the functionality of the MyD88 domain of the Jun.GHR.MyD88 ZipR. FIG. 5A shows transduction of the Ramos Blue MyD88 signaling reporter cell line with Jun.GHR and Jun.GHR.MyD88. FIG. 5B demonstrates that Jun. GHR.MyD88 activates MyD88 signaling in contrast to Jun. GHR.

### DETAILED DESCRIPTION

Despite recent advances in cancer treatment, patients with relapsed or refractory disease continue to have poor outcomes and novel approaches are needed. T cells that are genetically modified to express a chimeric antigen receptor (CAR) can kill chemotherapy-resistant tumor cells and therefore have the potential to improve outcomes and reduce treatment-related toxicity from conventional therapies [1, 2].

CARs typically consist of four components: i) an extracellular antigen recognition domain, most commonly a single chain variable fragment (scFv), ii) structural components, such as hinge and transmembrane domains, iii) a costimulatory domain that provides signals to sustain CAR T cell effector functions, and iv) a CD35 activation domain [1-3].

First generation CARs provide only signal 1, via CD35. Second-generation CARs also provide signal 2, most often through CD28 or 4-1BB co-stimulation to sustain CAR T cell expansion following activation. Although activated CAR T cells produce cytokines, such as interleukin-2 (IL-2), production decreases after repeated exposure to tumor cells [9], and some cytokines that are important for T cell effector function, such as IL-12 and IL-15, are either produced at low levels are not at all by T cells [23,24]. Due to these limitations, there is a need to engineer CAR T cells to augment cytokine-mediated signals.

The present application provides, among other things, chimeric receptors that can provide signals to the genetically modified immune cells. The chimeric receptors described herein comprise homodimeric polypeptides. Such chimeric receptors present a significant advance in the field of immunotherapy. Because only one polypeptide chain is required, the chimeric receptors described herein will simplify the generation of immune cells expressing the chimeric receptors and thus their clinical translation.

While data disclosed herein (see Examples section below) are focused on expressing CARs in T cells, methods and/or compositions of the present disclosure may be applicable to various other cell therapy platforms that are actively being explored including, but not limited to, NK cells, NK92 cells, NKT cells, and γδ T cells. Likewise, while the methods and/or compositions disclosed herein may be useful in the treatment of cancer, the present invention may also be broadly applicable to adoptive immunotherapies of non-malignant disease including, but not limited to, infectious diseases (e.g., viral infections) and autoimmune diseases.

### Definitions

The term “chimeric receptor” as used herein may refer to a cell-surface receptor that is engineered to have at least a portion of at least one domain (e.g., extracellular region, transmembrane region, intracellular region which can comprise, e.g., a signaling region, i.e., an intracellular signaling region) that is derived from sequences of one or more different origins.

The term “chimeric antigen receptor” or “CAR” as used herein is defined as a cell-surface receptor comprising an extracellular target-binding region, a transmembrane region, and an intracellular region comprising a lymphocyte activation domain and, optionally, at least one co-stimulatory signaling domain, all in a combination that is not naturally found together on a single protein. This particularly includes receptors wherein the extracellular region and the intracellular region are not naturally found together on a single receptor protein. The chimeric antigen receptors (CARs) of the present invention can be used with lymphocytes such as T cells and natural killer (NK) cells.

A “homodimerizing motif” or “homodimerization motif” is any molecule that has the ability to associate (covalently or non-covalently) with a molecule of the same constitution.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells,” which refer to a specialized population of T cells that express a semi-invariant αβ T cell receptor, but also express a variety of molecular markers that are typically associated with NK cells, such as NK1.1. NKT cells include NK1.1+ and NK1.1-, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to a specialized population of a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation and were found to be an important source of IL-17 and to induce robust CD8+ cytotoxic T cell response. Also included are “regulatory T cells” or “Tregs” which refers to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeably and used synonymously herein. As used herein, NK cell refers to a differentiated lymphocyte with a CD 16+CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample or might be a macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-recognition molecule” refers to any molecule that is capable of recognizing an antigen as described herein. Non-limiting examples of antigen-recognition molecules include T cell receptors (TCRs) (e.g., αβ TCRs), synthetic T cell receptors and antigen receptors (STARs), chimeric antigen receptor (CARs), T cell antigen couplers (TACs), T cell receptor fusion constructs (TruCs), HLA-independent T cell receptors (HITs), bispecific T cell engagers, and antibodies (e.g., bispecific antibodies) or antibody fragments.

Terms “antibody” and “antibodies” refer to monoclonal antibodies, multispecific antibodies (e.g., bispecific antibodies), human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgAQ1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used, or manipulated in any way, for the production of a substance by the cell, for example the expression by the cell of a gene, a DNA or RNA sequence, a protein, or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include immune cells (e.g., T cells and natural killer cells, or a macrophage) or stem cells (e.g., mesenchymal stem cells (MSCs), induced pluripotent stem (iPS) cells [iPSCs]) that contain the DNA or RNA sequences encoding a chimeric receptor described herein and express the chimeric receptor on the cell surface. Host cells may be used for enhancing immune cell activity (e.g., effector function), treatment of tumors, treatment of infectious diseases, and treatment of autoimmune disease.

The terms “activation” or “stimulation” means to induce a change in their biologic state by which the cells (e.g., T cells and NK cells) express activation markers, produce cytokines, proliferate, and/or become cytotoxic to target cells. All of these changes can be produced by primary stimulatory signals. Co-stimulatory signals can amplify the magnitude of the primary signals and suppress cell death following initial stimulation, resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular, or transmembrane.

The term “tumor” refers to a benign or malignant abnormal growth of tissue. The term “tumor” includes cancer.

The term “effector function” refers to a specialized function of a cell. Effector function of a T cell, for example, may be cytolytic activity (e.g., tumor killing activity) or helper activity including the secretion of cytokines.

As used herein, the term “safety switch” refers to any mechanism that is capable of removing or inhibiting the effect of a chimeric receptor described herein from a system (e.g., a culture or a subject).

The term “site-specific nuclease” as used herein refers to a nuclease capable of specifically recognizing and cleaving a nucleic acid (DNA or RNA) sequence.

The terms “genetically modified” or “genetically engineered” refers to the addition of extra genetic material in the form of DNA or RNA into a cell.

As used herein, the term “derivative” or “derived from” in the context of proteins or polypeptides (e.g., chimeric receptors or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of; (d) a polypeptide encoded by a nucleotide sequence that can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin).

Information regarding hybridization conditions (e.g., high, moderate, and typical stringency conditions) have been described, see, e.g., U.S. Patent Application Publication No. US 2005/0048549 (e.g., paragraphs 72-73) the contents of which is incorporated herein by reference in its entirety for all purposes.

The term “portion” when used in reference to a polypeptide or protein, refers to any component of the polypeptide or protein, including but not limited to, a subunit, a domain, or a fragment. For example, a portion of a dimeric cell-surface receptor can be a subunit (or a chain) the receptor.

The term “functional fragment” as used herein refers to a fragment of the polypeptide or protein, or a polynucleotide encoding the polypeptide or protein, that retains at least one function of the full-length polypeptide or protein. A functional fragment may comprise one, two, three, or more fragments of the full-length polypeptide or protein, or polynucleotide encoding the polypeptide or protein. Each fragment may comprise an amino acid sequence of at least 5 contiguous amino acid residues, at least 6 contiguous amino acid residues, at least 7 contiguous amino acid residues, at least 8 contiguous amino acid residues, at least 9 contiguous amino acid residues, at least 10 contiguous amino acid residues, at least 11 contiguous amino acid residues, at least 12 contiguous amino acid residues, at least 13 contiguous amino acid residues, at least 14 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of the full-length polypeptide or protein.

The terms “vector,” “cloning vector,” “recombinant vector,” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, a retroviral vector, a lentiviral vector, an adenoviral vector, an adeno-associated virus vector, an alphaviral vector, a herpes virus vector, a baculoviral vector, and a vaccinia virus vector.

As used herein, the term “operably linked,” or “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

As used herein, the term “promoter” refers to any sequence(s) capable of driving transcription of a coding sequence in a cell. Therefore, promoters used in the vectors of the disclosure may comprise cis-acting transcriptional control elements and regulatory sequences that are involved in regulating or modulating the timing and/or rate of transcription of a gene. By way of a non-limiting example, a promoter can be a cis-acting transcriptional control element, including an enhancer, a promoter, a transcription terminator, an origin of replication, a chromosomal integration sequence, 5′ and 3′ untranslated regions, or an intronic sequence, which participate in transcriptional regulation. Such cis-acting sequences typically interact with proteins or other biomolecules to carry out (turn on/off, regulate, modulate, etc.) transcription. “Constitutive” promoters are those that drive expression continuously under the majority of environmental conditions and states of cellular development and/or differentiation. “Inducible” or “regulatable” promoters direct expression of a nucleic acid under the influence of developmental and/or environmental. Non-limiting examples of environmental conditions that may influence transcription via inducible promoters include elevated temperature, drought, the presence of light, and anaerobic conditions.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in immune cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disease, disorder or condition include: (1) preventing, delaying, or reducing the incidence and/or likelihood of the appearance of at least one clinical or sub-clinical symptom of the state, disease, disorder or condition developing in a subject that may be afflicted with or predisposed to the state, disease, disorder or condition, but does not yet experience or display clinical or subclinical symptoms of the state, disease, disorder or condition; or (2) inhibiting the state, disease, disorder or condition, i.e., arresting, reducing or delaying the development of the state, disease, disorder or condition or a relapse thereof or at least one clinical or sub-clinical symptom thereof; or (3) relieving the state, disease, disorder or condition, i.e., causing regression of the state, disease, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “pharmaceutical composition,” as used herein, represents a composition comprising polynucleotides, vectors, peptides, compositions, or host cells described herein formulated for administration to a subject for treatment, abatement, or prevention of a disease.

The phrase “pharmaceutically acceptable,” as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” as used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid,” “nucleotide,” and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The terms “patient,” “individual,” “subject,” and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising”, or versions thereof (e.g., comprises), a feature, embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Receptor

In some embodiments, the present disclosure provides a chimeric receptor which is a homodimer comprising two polypeptide monomers, wherein each polypeptide monomer may comprise:
i. an extracellular region comprising at least one homodimerizing motif;
ii. a transmembrane region; and
iii. an intracellular region comprising at least one signaling region.

In some embodiments, the at least one signaling region may be derived from a cell-surface receptor or a signal transducing adaptor protein, or a portion or a variant thereof.

### Extracellular Region

In some embodiments, the extracellular region of the chimeric receptor may comprise at least one homodimerizing motif. In some embodiments, the extracellular region of the chimeric receptor may comprise more than one homodimerizing motif, such as two, three, four, five or more homodimerizing motifs.

In some embodiments, the homodimerizing motif may be a homodimerizing leucine zipper motif, a ligand-dependent homodimerizing motif, or a homodimerizing motif that can otherwise bring the signaling region in close proximity (e.g., single chain variable fragment).

In some embodiments, the extracellular region may comprise at least one leucine zipper motif. In some embodiments, the extracellular region may comprise more than one leucine zipper motif, such as two, three, four, five or more leucine zipper motifs.

In some embodiments, the leucine zipper motif may comprise at least two, three, four, five heptad repeats of amino acids with a leucine at every seventh position. In some embodiments, the leucine zipper motif may comprise at least five heptad repeats of amino acids with a leucine at every seventh position.

In some embodiments, the leucine zipper motif may be derived from transcription factor c-Jun. In some embodiments, the leucine zipper motif may comprise the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the nucleotide sequence that encodes the leucine zipper motif comprises the nucleotide sequence that encodes the amino acid sequence of SEQ ID NO: 9, or a variant thereof having at least 50%, at least 55%, at least 60%, at least 65%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the leucine zipper motif is encoded by the nucleotide sequence of: SEQ ID NO: 3, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In some embodiments, the leucine zipper motif comprises the amino acid sequence set forth in SEQ ID NO: 9. In some embodiments, the nucleotide sequence that encodes the leucine zipper motif comprises the nucleotide sequence set forth in SEQ ID NO: 3.

In some embodiments, the leucine zipper motif may comprise any homodimerizing leucine zipper motif as described, for example, in Moll et al., 2001 [6], Reike et al., 2013 [7], and Newman et al., 2003 [8], the contents of each of which is incorporated herein by reference in its entirety for all purposes.

In certain embodiments, zipper motifs, e.g., leucine zipper motifs, which may be used in accordance with the present disclosure may be derived from any number of human proteins within the knowledge of one skilled in the art. Non-limiting examples of human proteins comprising zipper motifs that may be used in accordance with the present disclosure are described in Table 1. Regarding Table 1 (see also Newman J R S and Keating A E. Comprehensive identification of human bZIP interactions with coiled-coil arrays. Science. 2003 Jun. 27; 300 (5628): 2097-101, incorporated herein by reference in its entirety for all intended purposes), a: Other names for a protein specified at left, or names of other proteins sharing the same coiled coil sequence used herein. b: The sequences used in the array experiments are divided into five sections. From left to right: [cloning vector sequence]-[basic region included for cloning]-[coiled-coil domain]-[additional resides to facilitate cloning]-[cloning vector]. The N-terminal end of the coiled-coil domain (at left) was determined by reference to the Fos/Jun crystal structure, the C-terminal end by the point at which the PAIRCOIL probability drops below 10%. The register of the coiled-coil domain is shown at the bottom of the column starting at the f position. Sequences were aligned according to sequence similarity of the coiled-coil domain using CLUSTALX. Additional protein sequences denoted by the numbers inside the square brackets are as follows: [1] through [4]-MSYYHHHHHHLESTSLYKKA (SEQ ID NO: 207), followed by GSGS (SEQ ID NO: 208) [1], GSEF (SEQ ID NO: 209) [2], GSGR (SEQ ID NO: 210) [3] and GFDD (SEQ ID NO: 211) [4]; [5]-LE; [6]-KVE; [7]-QLE; [8]-RLE; [9]-E; [10]-FLE; [11]-YLEII; [12]-ALE; [13]-NLE; [14]-CSSNTQLSCTKWLIRGC (SEQ ID NO: 213); [15]-SSNTQLSCTKWLIRGC (SEQ ID NO: 214); [16]-HLE; [17]-STCRDL (SEQ ID NO: 215); [18]-FSTCRDL (SEQ ID NO: 216); [19]-LDLQRSMNRRY (SEQ ID NO: 217); [20]-PAEIYES (SEQ ID NO: 218). The initial methionine in sequences [1] through [4] is cleaved in vivo during expression.

TABLE 1
Non-Limiting Examples of Human Proteins Comprising Zipper Motifs

| | | | | SEQ |
|---|---|---|---|---|
| | | | | ID |
| Family | Name | Aliasesª | Protein Constructs^{b} | NO. |
| ATF-2 | ATF-2 | CREB-2, | [2]-DLERNRAAASRCRQKRK- | 55 |
| | | CREBP1, | VWVQSLEKKAEDLSSLNGQLQSEVTLLRNEVAQLK | |
| | | HB16, | QLLLAHKDC-PVTAMQKKSG-[10] | |
| | | TCRATF2 | | |
| ATF-2 | ATF-7 | ATFa | [2]-KRK- | 56 |
| | | | LWVSSLEKKAEELTSQNIQLSNEVTLLRNEVAQLKQ | |
| | | | LLLAHKDC-PVTALQK-[5] | |
| ATF-2 | CREBPA | CBREPA | [2]-QRK- | 57 |
| | | | VWVMSLEKKAEELTQTNMQLQNEVSMLKNEVAQL | |
| | | | KQLLLTHKDC-PITAMQKES-[5] | |
| ATF-6 | ATF-6 | ATF6alpha | [2]-ACQSRKKKK- | 58 |
| | | | EYMLGLEARLKAALSENEQLKKENGTLKRQLDEVV | |
| | | | SENQRLKV-PSPKRRV-[5] | |
| ATF-6 | CREBL1 | ATF6beta, | EYLQGLEARLQAVLADNQQLRRENAALRRRLEALL | 59 |
| | | CREB-RP | AENSELKLGSGNRK | |
| ATF-6 | ZF | Zhangfei | [2]-CRLNRLKKK- | 60 |
| | | | EYVMGLESRVRGLAAENQELRAENRELGKRVQALQ | |
| | | | EESRYLRAVLANETGL-[]-[9] | |
| B-ATF | B-ATF | p21-SNFT | [1]-[]- | 61 |
| | | | QKADTLHLESEDLEKQNAALRKEIKQLTEELKYFTS | |
| | | | VLNSHE-[]-[5] | |
| B-ATF | p21SNFT | — | [2]-RSRKKQT- | 62 |
| | | | QKADKLHEEYESLEQENTMLRREIGKLTEELKHLTE | |
| | | | ALKEHEKMC-PLLLCPMNFVH-[5] | |
| B-ATF | BAC04846 | — | DKADALHQQHESLEKDNLALRKEIQSLQAELAWWS | 63 |
| | | | RTLHVHE | |
| C/EBP | C/EBPα | C/EBP | [1]-[]- | 64 |
| | | | QRNVETQQKVLELTSDNDRLRKRVEQLSRELDTLRG | |
| | | | IFRQL-[]-[5] | |
| C/EBP | C/EBPβ | NF-IL6, TF 5, | [2]-SDEYKIRRERNNIAVRKSRDKAK- | 65 |
| | | LAP, CRP2, | MRNLETQHKVLELTAENERLQKKVEQLSRELSTLRN | |
| | | LIP, AGP/EBP, | LFKQL-PEPLLASSGHC-[] | |
| | | FN-M, GPE1- | | |
| | | BP | | |
| C/EBP | C/EBPγ | Ig/EBP-1 | [2]-GERNNMAVKKSRLKSK- | 66 |
| | | | QKAQDTLQRVNQLKEENERLEAKIKLLTKELSVLKD | |
| | | | LFLEHAHNLAD-NVQSISTENTTADG-[5] | |
| C/EBP | C/EBPδ | NFIL6, | [1]-[]- | 67 |
| | | NFIL 6beta | RRNQEMQQKLVELSAENEKLHQRVEQLTRDLAGLR | |
| | | | QFFKQL-[]-[5] | |
| C/EBP | C/EBPε | — | [2]-HLEYRLRRERNNIAVRKSRDKAK- | 68 |
| | | | RRILETQQKVLEYMAENERLRSRVEQLTQELDTLRN | |
| | | | LFRQI-PEAANLIKGV-[5] | |
| C/EBP | CHOP | DDIT3, | [2]-RMK- | 69 |
| | | GADD153 | EKEQENERKVAQLAEENERLKQEIERLTREVEATRR | |
| | | | ALIDRMVNLHQA-[]-[] | |
| CNC | NFE2 | p45 NF-E2 | [2]-QRKL- | 70 |
| | | | ETIVQLERELERLTNERERLLRARGEADRTLEVMRQ | |
| | | | QLTELYR-DIFQHLRDESGNS-[] | |
| CREB | ATF-1 | TREB36, TREB, | [2]-DPQLKREIRLMKNREAARECRRKKK- | 71 |
| | | TCRATF1 | EYVKCLENRVAVLENQNKTLIEELKTLKDLYSNKSV- | |
| | | | []-[] | |
| CREB | CREM-Ia | HCREM-1; | [1]-KEEATRKRELRLMKNREAARECRRKKK- | 72 |
| | | CREMα, τα; | EYVKCLENRVAVLENQNKTLIEELKALKDLYCHKVE- | |
| | | ICER1, 1γ | []-[] | |
| CREB | CREM- | possible | [1]-KEEATRKRELRLMKNREAARECRRKKK- | 73 |
| | Ia(K324R) | sequence | EYVKCLENRVAVLENQNKTLIEELKALRDLYC-[]-[7] | |
| | | variant | | |
| | | (AAC60616 | | |
| | | numbering) | | |
| CREB | CREM-Ib | CREMβ, γ, , | [1]-[]-EYVKCLESRVAVLEVQNKKLIEELETLKDICS- | 74 |
| | | τ1, τ2, τα; | []-[5] | |
| | | ICER1, 1γ, 2γ, | | |
| | | 3 | | |
| CREB | CREB | CREBδ, | [1]-KEAARKREVRLMKNREAARECRRKKK- | 75 |
| | | CREB-A, | EYVKCLENRVAVLENQNKTLIEELKALKDLYCHKSD- | |
| | | CREB327, | []-[] | |
| | | CREB-1 | | |
| E4BP4 | E4BP4 | NFIL3, NF- | [2]-EKRR- | 76 |
| | | IL3A | LNDLVLENKLIALGEENATLKAELLSLKLKFGLIS- | |
| | | | STAYAQEIQKLSNSTAVYFQDYQTSKSNV-[5] | |
| E4BP4 | AC012616 | | LNDAAIAGRLAALMEENALLKGELKALKLRFGLLPL | 77 |
| | | | T | |
| Jun | cJun | AP-1 | [2]-QRKL- | 78 |
| | | | ERIARLEEKVKTLKAQNSELASTANMLREQVAQLKQ | |
| | | | KVMNH-VNSGCQLMLTQQY-[5] | |
| IgMAF | cMaf | RIPE3b1 | [2]-QRV- | 79 |
| | | | QQRHVLESEKNQLLQQVDHLKQEISRLVRERDAYKE | |
| | | | KYEKLVSSGFREN-GSSSDNPSSPEFFM-[] | |
| IgMAF | MafB | — | [2]-QYKRV- | 80 |
| | | | QQKHHLENEKTQLIQQVEQLKQEVSRLARERDAYK | |
| | | | VKCEKLANSG-FREAGSTSDSPSSPEFFL-[] | |
| IgMAF | MafA | — | QQRHILESEKCQLQSQVEQLKLEVGRLAKERDLYKE | 81 |
| | | | KYEKLAGRGG | |
| IgMAF | NRL | — | QQRRGLEAERARLAAQLDALRAEVARLARERDLYK | 82 |
| | | | ARCDRLTSSG | |
| OASIS | OASIS | LZIP, LZIP- | [1]-[]- | 83 |
| | | alpha, luman | EYVECLEKKVETFTSENNELWKKVETLENANRTLLQ | |
| | | | QLQKLQTLVTNKISR-[]-[5] | |
| OASIS | CREB-H | AIBZIP | [1]-[]- | 84 |
| | | | EYIDGLETRMSACTAQNQELQRKVLHLEKQNLSLLE | |
| | | | QLKKLQAIVVQSTS-[]-[5] | |
| OASIS | CREB3 | — | [2]-WRRKIRNKRSAQESRRKKK- | 85 |
| | | | VYVGGLESRVLKYTAQNMELQNKVQLLEEQNLSLL | |
| | | | DQLRKLQAMVIEISNKTSS-[]-[8] | |
| OASIS | CREB4 | — | [2]-WRKIRNKQSAQDSRRRKK- | 86 |
| | | | EYIDGLESRVAACSAQNQELQKKVQELERHNISLVA | |
| | | | QLRQLQTLIAQTSNKAAQTST-CVL-[5] | |
| OASIS | AC022173 | — | EYMDSLEDDVESCSTENLELRKKVEVLENTNRASHG | 87 |
| | | | ELV | |
| PAR | DBP | TAXREB302 | [1]-EEQKDEKYWSRRYKNNEAAKRSRDARR- | 88 |
| | | | LKENQISVRAAFLEKENALLRQEVVAVRQELSHYRA | |
| | | | VLSRYQAQ-HGAL-[] | |
| PAR | HLF | — | [3]-[]- | 89 |
| | | | LKENQIAIRASFLEKENSALRQEVADLRKELGKCKNI | |
| | | | LAKYEARH-[]-[5] | |
| PAR | TEF | TEF | [2]-NWTRRKKNNVAAKRSRDARR- | 90 |
| | | | LKENQITIRAAFLEKENTALRTEVAELRKEVGKCKTI | |
| | | | VSKYETK-YGPL-[] | |
| smMAF | MafG | hMaf | [2]-GV- | 91 |
| | | | TQKEELEKQKAELQQEVEKLASENASMKLELDALRS | |
| | | | KYEALQTFARTVARS- | |
| | | | PVAPARGPLAAGLGPLVPGKVAATSVITIVKSKTDA- | |
| | | | [5] | |
| smMAF | MafK | NF-E2 p18 | [1]-[]- | 92 |
| | | subunit | TQKEELERQRVELQQEVEKLARENSSMRLELDALRS | |
| | | | KYEALQTFARTVAR-[]-[5] | |
| smMAF | MafF | — | CQKEELQKQKSELEREVDKLARENAAMRLELDALR | 93 |
| | | | GKCEALQGFARSVAAARG | |
| XBP | XBP-1 | TREB5, TREB | [2]-QTARDRKK- | 94 |
| | | | ARMSELEQQVVDLEEENQKLLLENQLLREKTHGLV | |
| | | | VENQEL-RQRLGMDALVAEEEAEAKGNEV-[5] | |
| YEASTe | ARR1 | YAP8, ACR1, | [2]-PSQNAFRKRKL- | 95 |
| | | YPR199C | ERLEELEKKEAQLTVINDQIHILKKENELLHF- | |
| | | | MLRSLLTERNMP-[14] | |
| YEASTe | CIN5 | YAP4, | [2]-KRAAQNRSAQKAFRQRRE- | 96 |
| | | YOR028C | KYIKNLEEKSKLFDGLMKENSELKKMIESLKSKLKE- | |
| | | | []-[] | |
| YEASTe | YAP2 | CAD1, | [4]- | 97 |
| | | YDR423C | EDTPPNPKKEIEFPGGSPGRKRIDSEAKSRRTAQNRA | |
| | | | AQRAFRDRKE-AKMKSLQERVELLEQK- | |
| | | | DAQNKTTTDFLLCSLKSLLSEITKYRAKNSDDERILA | |
| | | | FLDDL-[16] | |
| YEASTe | YAP3 | YHL009C | [4]-EDTPPNPKKEIEFQAKKKAQNRAAQKAFRERKE- | 98 |
| | | | ARMKELQDKLLESERNRQSLLKEIEELRKANTEINAE | |
| | | | NRLLLRSGN-[]-[17] | |
| YEASTe | YAP5 | YIR018W | [4]-EDTPPNPKKEIEFPKRQNRDAQRAYRERKN- | 99 |
| | | | NKLQVLEETIESLSKVVKNYETKLNRLQNELQAKES | |
| | | | ENHALKQKLETLTLKQASV-PAQDPILQN-[18] | |
| YEASTe | YAP7 | YOL028C | [2]-QQNRDAQRAYRERRT- | 100 |
| | | | TRIQVLEEKVEMLHNLVDDWQRKYKLLESEFSDTKE | |
| | | | NLQKSIALNNELQKAL-PLIVNTPFQQQPEN-[15] | |
| YEASTe | GCN4 | ARG9, AAS3, | [4]- | 101 |
| | | YEL009C | EDTPPNPKKEIEFHPAALKRARNTEAARRSRARKL- | |
| | | | QRMKQLEDKVEELLSKNYHLENEVARLKKLVGDAA | |
| | | | R-[]-[20] | |
| YEASTe | MET4 | YNL 103W | [4]-EDTPPNPKKEIEFDDDEDLLIQSKKSHQKKK- | 102 |
| | | | LKEKELESSIHELTEIAASLQKRIHTLETENKLLKNLV | |
| | | | LSSGET-[]-[18] | |
| Basic | Basic | Basic region | basic region- | — |
| region | region | | fgabcdefgabcdefgabcdefgabcdefgabcdefgabcdefgabcde | |
| | | | fgabcdefgabcdefg | |

In some embodiments, the extracellular region of the chimeric receptor disclosed herein may comprise at least one ligand-dependent homodimerizing motif. For example, the ligand-dependent homodimerizing motif may comprise a chemical inducer of dimerization (CID)-binding domain such as an FKBP12 domain (see Mata et al., 2017 [9], Juillerat et al., 2019 [17], Leung et al., 2019 [5], the contents of each of which is incorporated herein by reference in its entirety for all purposes). In one embodiment, the ligand-dependent homodimerizing motif comprises an FKBP12ᵥ₃₆ domain. In one embodiment, the ligand-dependent homodimerizing motif comprises a single chain variable fragment.

In some embodiments, the extracellular region of the chimeric receptor disclosed herein may comprise other homodimerizing motifs which can bring the signaling domain in close proximity, such as a single chain variable fragment as a non-limiting example (see, e.g., Katsarou et al., 2021 [10], the contents of which is incorporated herein by reference in its entirety for all purposes). A single chain variable fragment can induce homodimerization by binding a single ligand, which may be another example of a “ligand dependent homodimerizing motif”, or it may induce homodimerization by hydrophobic interactions between two variable fragments.

In some embodiments, where two or more homodimerizing motifs are used, the two or more homodimerizing motifs may be operatively linked to each other via any of various linkers. Example linker sequences can comprise (GGGGS)n (SEQ ID NO: 150), wherein n is any integral number (e.g., 1, 2, 3, 4, 5).

In some embodiments, the linker comprises the amino acid sequence of SEQ ID NO: 151, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 151. In some embodiments, the nucleotide sequence that encodes the linker comprises the nucleotide sequence that encodes the amino acid sequence of SEQ ID NO: 151, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 151. In some embodiments, the nucleotide sequence that encodes the linker comprises the nucleotide sequence set forth in SEQ ID NO: 152, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 152. In some embodiments, the nucleotide sequence that encodes the linker comprises the nucleotide sequence set forth in SEQ ID NO: 153, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 153. In some embodiments, the linker comprises the amino acid sequence set forth in SEQ ID NO: 151. In some embodiments, the nucleotide sequence that encodes linker comprises the nucleotide sequence set forth in SEQ ID NO: 152. In some embodiments, the nucleotide sequence that encodes linker comprises the nucleotide sequence set forth in SEQ ID NO: 153.

### Hinge Region

In some embodiments, the extracellular region of a chimeric receptor disclosed herein may comprise a hinge region.

The hinge region may be derived from all or part of naturally occurring molecules, such as from all or part of the extracellular region of CD8, CD4, or CD28, or from all or part of an antibody constant region. Alternatively, the hinge region may be a synthetic sequence that corresponds to a naturally occurring hinge region sequence or may be an entirely synthetic hinge region sequence.

Non-limiting examples of hinge regions which may be used in accordance with the invention include a part of human CD8α, partial extracellular region of CD28, FcyRllla receptor, IgG, IgM, IgA, IgD, IgE, an Ig hinge, or functional fragment thereof. The hinge may be mutated to prevent Fc receptor binding. The hinge region can be derived from CD8α stalk, CD28, or IgG1. In certain embodiments, the hinge region is derived from CD8α stalk. In various embodiments, the hinge region is derived from CD28. The hinge region can provide flexibility and accessibility between the extracellular region and the transmembrane region.

In some embodiments, the hinge may be derived, for example, from IgG1, IgG2, IgG3, IgG4, CD28, or CD8α.

In some embodiments, the hinge region may be derived from the same cell-surface receptor as the intracellular signaling region. In some embodiments, the hinge region may be derived from a molecule different from the cell-surface receptor from which the intracellular signaling region is derived.

The hinge region may comprise up to 300 amino acids, from 10 to 100 amino acids, or from 25 to 50 amino acids.

### Leader Sequence

In various embodiments, the extracellular region of the chimeric receptor disclosed herein may comprise a leader sequence. The leader sequence may be positioned at the N-terminus of the extracellular region. The leader sequence may be optionally cleaved from the extracellular region during cellular processing and localization of the chimeric receptor to the cellular membrane. Any of various leader sequences known to one of skill in the art may be used as the leader sequence. Non-limiting examples of peptides from which the leader sequence may be derived include FcεR, human immunoglobulin heavy chain variable region, CD8α, or any of various other proteins secreted by T cells. In various embodiments, the leader sequence is compatible with the secretory pathway of a T cell. In certain embodiments, the leader sequence is derived from human immunoglobulin heavy chain.

In some embodiments, the leader sequence may be derived from an immunoglobulin heavy chain variable region or colony stimulating factor 2 receptor alpha chain (CSF2RA). In some embodiments, the leader sequence may be derived from an immunoglobulin heavy chain variable region.

In some embodiments, the leader sequence comprises the amino acid sequence of SEQ ID NO: 7, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 7. In some embodiments, the nucleotide sequence that encodes the leader sequence comprises the nucleotide sequence that encodes the amino acid sequence of SEQ ID NO: 7, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 7. In some embodiments, the nucleotide sequence that encodes the leader sequence comprises the nucleotide sequence set forth in SEQ ID NO: 1, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In some embodiments, the leader sequence comprises the amino acid sequence set forth in SEQ ID NO: 7. In some embodiments, the nucleotide sequence that encodes the leader sequence comprises the nucleotide sequence set forth in SEQ ID NO: 1.

### Transmembrane Region

In some embodiments, chimeric receptor disclosed herein comprises a transmembrane region.

In some embodiments, the transmembrane region may be derived from the same cell-surface receptor as the intracellular signaling region.

In some embodiments, the transmembrane domain may be derived from EpoR, or a portion or variant thereof.

In some embodiments, the transmembrane region derived from EpoR may comprise the amino acid sequence of SEQ ID NO: 48, SEQ ID NO: 104, SEQ ID NO: 128, SEQ ID NO: 106, SEQ ID NO: 130, SEQ ID NO: 108, or SEQ ID NO: 132, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48, SEQ ID NO: 104, SEQ ID NO: 128, SEQ ID NO: 106, SEQ ID NO: 130, SEQ ID NO: 108, or SEQ ID NO: 132. In some embodiments, the nucleotide sequence that encodes the transmembrane region derived from EpoR comprises the nucleotide sequence that encodes the amino acid sequence of SEQ ID NO: 48, SEQ ID NO: 104, SEQ ID NO: 128, SEQ ID NO: 106, SEQ ID NO: 130, or SEQ ID NO: 108, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48, SEQ ID NO: 104, SEQ ID NO: 128, SEQ ID NO: 106, SEQ ID NO: 130, or SEQ ID NO: 108, or SEQ ID NO: 132. In some embodiments, the transmembrane region derived from EpoR may be encoded by the nucleotide sequence of SEQ ID NO: 103, SEQ ID NO: 105, SEQ ID NO: 127, SEQ ID NO: 107, SEQ ID NO: 129, SEQ ID NO: 109, or SEQ ID NO: 131, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103, SEQ ID NO: 105, SEQ ID NO: 127, SEQ ID NO: 107, SEQ ID NO: 129, SEQ ID NO: 109, or SEQ ID NO: 131. In some embodiments, the transmembrane region derived from EpoR comprises the amino acid sequence set forth in SEQ ID NO: 48, SEQ ID NO: 104, SEQ ID NO: 128, SEQ ID NO: 106, SEQ ID NO: 130, SEQ ID NO: 108, or SEQ ID NO: 132. In some embodiments, the nucleotide sequence that encodes the transmembrane region derived from EpoR comprises the nucleotide sequence set forth in SEQ ID NO: 103, SEQ ID NO: 105, SEQ ID NO: 127, SEQ ID NO: 107, SEQ ID NO: 129, SEQ ID NO: 109, or SEQ ID NO: 131.

In some embodiments, the transmembrane region may be derived from GHR, or a portion or variant thereof.

In some embodiments, the transmembrane region derived from GHR may comprise the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52, SEQ ID NO: 134, SEQ ID NO: 136, SEQ ID NO: 138, SEQ ID NO: 140, SEQ ID NO: 142, or SEQ ID NO: 144. In some embodiments, the nucleotide sequence that encodes the transmembrane region derived from GHR comprises the nucleotide sequence that encodes the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52, SEQ ID NO: 134, SEQ ID NO: 136, SEQ ID NO: 138, SEQ ID NO: 140, SEQ ID NO: 142, or SEQ ID NO: 144. In some embodiments, the transmembrane region derived from GHR may be encoded by the nucleotide sequence of SEQ ID NO: 133, SEQ ID NO: 135, SEQ ID NO: 137, SEQ ID NO: 139, SEQ ID NO: 141, SEQ ID NO: 143, SEQ ID NO: 145, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 133, SEQ ID NO: 135, SEQ ID NO: 137, SEQ ID NO: 139, SEQ ID NO: 141, SEQ ID NO: 143, or SEQ ID NO: 145. In some embodiments, the transmembrane region derived from EpoR comprises the amino acid sequence set forth in SEQ ID NO: 52, SEQ ID NO: 134, SEQ ID NO: 136, SEQ ID NO: 138, SEQ ID NO: 140, SEQ ID NO: 142, or SEQ ID NO: 144. In some embodiments, the nucleotide sequence that encodes the transmembrane region derived from EpoR comprises the nucleotide sequence set forth in SEQ ID NO: 133, SEQ ID NO: 135, SEQ ID NO: 137, SEQ ID NO: 139, SEQ ID NO: 141, SEQ ID NO: 143, or SEQ ID NO: 145.

In some embodiments, the transmembrane domain may be derived from IL-18Ra, IL-18RB, CD28, IL-2RB, CD8, CD4, CD3ζ, CD40, CD134 (OX-40), CD19, or CD7.

In some embodiments, the transmembrane domain may be derived from IL-18Ra, or a portion or variant thereof.

In some embodiments, the transmembrane region derived from IL-18Ra may comprise the amino acid sequence of SEQ ID NO: 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40. In some embodiments, the nucleotide sequence that encodes the transmembrane region derived from IL-18Ra comprises the nucleotide sequence that encodes the amino acid sequence of SEQ ID NO: 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40. In some embodiments, the transmembrane region derived from IL-18Ra may be encoded by the nucleotide sequence of SEQ ID NO: 39, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 39. In some embodiments, the transmembrane region derived from IL-18Ra comprises the amino acid sequence set forth in SEQ ID NO: 40. In some embodiments, the nucleotide sequence that encodes the transmembrane region derived from IL-18Ra comprises the nucleotide sequence set forth in SEQ ID NO: 39.

In some embodiments, the transmembrane region may be derived from IL-18RB, or a portion or variant thereof.

In some embodiments, the transmembrane region derived from IL-18RB may comprise the amino acid sequence of SEQ ID NO: 36, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 36. In some embodiments, the nucleotide sequence that encodes the transmembrane region derived from IL-18RB comprises the nucleotide sequence that encodes the amino acid sequence of SEQ ID NO: 36, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 36. In some embodiments, the transmembrane region derived from IL-18RB may be encoded by the nucleotide sequence of SEQ ID NO: 35, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35. In some embodiments, the transmembrane region derived from IL-18RB comprises the amino acid sequence set forth in SEQ ID NO: 36. In some embodiments, the nucleotide sequence that encodes the transmembrane region derived from IL-18RB comprises the nucleotide sequence set forth in SEQ ID NO: 35.

In some embodiments, the transmembrane region may be derived from CD28, or a portion or variant thereof.

In some embodiments, the transmembrane region derived from CD28 may comprise the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In some embodiments, the nucleotide sequence that encodes the transmembrane region derived from CD28 comprises the nucleotide sequence that encodes the amino acid sequence of SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In some embodiments, the transmembrane region derived from CD28 may be encoded by the nucleotide sequence of SEQ ID NO: 41, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 41. In some embodiments, the transmembrane region derived from CD28 comprises the amino acid sequence set forth in SEQ ID NO: 42. In some embodiments, the nucleotide sequence that encodes the transmembrane region derived from CD28 comprises the nucleotide sequence set forth in SEQ ID NO: 41.

In certain embodiments, the transmembrane region is derived from CD8α, CD28, CD8, CD4, CD3ζ, CD40, CD134 (OX-40), CD19, or CD7. In a specific embodiment, the transmembrane region is derived from CD8α. In a specific embodiment, the transmembrane region is derived from CD28. The transmembrane region may be fused in frame or operably linked between the extracellular region and the intracellular signaling region.

In some embodiments, the transmembrane region can be modified by amino acid deletions or additions for optimized intracellular orientation. In some embodiments, the transmembrane region may be modified by 1, 2, 3, 4, 5, or 6 amino acid deletions or additions for optimized intracellular orientation. For example, the transmembrane region described herein may be modified by 1, 2, 3, 4, 5, or 6 amino acid deletions either at the N-terminus or C-terminus of the transmembrane region. For example, the transmembrane region described herein may be modified by 1, 2, 3, 4, 5, or 6 amino acid additions (e.g., alanine additions) either at the N-terminus or C-terminus of the transmembrane region.

In some instances, the transmembrane region can be modified by amino acid substitution, deletions, or insertions to avoid binding of proteins naturally associated with the transmembrane region. In certain embodiments, the transmembrane region includes additional amino acids to allow for flexibility and/or optimal distance between the regions connected to the transmembrane region.

The transmembrane region may be derived from a natural or from a synthetic source. Where the source is natural, the region may be derived from any membrane-bound or transmembrane protein. Non-limiting examples of transmembrane regions of particular use in this disclosure may be derived from (i.e., comprise at least the transmembrane region(s) of) the a, B or (chain of the T cell receptor, CD28, CD38, CD35, CD45, CD4, CD5, CD7, CD8, CD8α, CD9, CD16, CD22, CD33, CD37, CD40, CD64, CD80, CD86, CD134 (OX-40), CD137, or CD154. Alternatively, the transmembrane region may be synthetic, in which case the transmembrane region will comprise predominantly hydrophobic residues such as leucine and valine. For example, a triplet of phenylalanine, tryptophan and/or valine can be found at each end of a synthetic transmembrane region.

In some embodiments, it will be desirable to utilize the transmembrane region of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane region will be selected or modified by amino acid substitution to avoid-binding of such regions to the transmembrane regions of the same or different surface membrane proteins to minimize interactions with other members of the receptor complex. In other cases, it will be desirable to employ the transmembrane region of ζ, η or FcεR1γ and -β, MB1 (Igα.), B29 or CD3-γ, ζ, or η, in order to retain physical association with other members of the receptor complex.

### Intracellular Signaling Region

In some embodiments, the chimeric receptor disclosed herein may comprise an intracellular region comprising at least one signaling region (also referred to herein as an intracellular signaling region). The at least one intracellular signaling region may be derived from a cell-surface receptor or a signal transducing adaptor protein, or a portion or variant thereof.

In some embodiments, the intracellular region may comprise more than one signaling region, for example, two, three, four or more signaling regions. In some embodiments, the intracellular region may comprise a first signaling region derived from a cell-surface receptor, or a portion or variant thereof, and a second signaling region derived from a signal transducing adaptor protein, or a portion or variant thereof.

In some embodiments, the signaling region is derived from a natural homodimeric cell-surface receptor or signal transducing adaptor protein. In some embodiments, the signaling region is derived from a non-natural homodimeric cell surface receptor or signal transducing adaptor protein.

In some embodiments, the signaling region derived from a cell surface receptor or signal transducing adaptor protein, or a portion or variant thereof, can activate Janus kinase (JAK)-signal transducer and activator of transcription (STAT) pathway, nuclear factor kappa-light-chain-enhancer of activated B cells (NFκB) pathway, and/or additional signaling pathways activated by the MyD88 signaling complex (Myddosome).

In some embodiments, the additional signaling pathways activated by the MyD88 signaling complex (Myddosome) are one or more of interleukin-1 receptor-associated kinase (IRAK) (e.g., IRAK1, IRAK2, IRAK4), TNF receptor (TNFR)-associated factor (TRAF) (e.g., TRAF6), TANK-binding kinase (TBK) (e.g., TBK1), mitogen-activated protein kinase (MAPK), Protein kinase B or AKT (PKB/AKT), and/or phosphatidylinositol-3-kinase (PI3K) pathways (see, e.g., Fitzgerald and Kagan, Cell 180, Mar. 19, 2020, the contents of which is incorporated herein by reference in its entirety for all purposes).

In some embodiments, the signaling region may be derived from a cytokine receptor, or a portion or variant thereof. In some embodiments, the signaling region may be derived from a natural homodimeric cytokine receptor. In some embodiments, the signaling region may be derived from a non-natural homodimeric cytokine receptor.

In some embodiments, the cytokine receptor may be erythropoietin receptor (EpoR), growth hormone receptor (GHR), prolactin receptor (PRLR), leptin receptor (LEPR), granulocyte colony stimulating factor (G-CSFR), thrombopoietin receptor (TpoR), interleukin-23 receptor (IL-23R), interleukin 10 receptor beta subunit (IL-10R2), IL-6 beta chain (gp130), IL-2RB, IL-18Ra, or IL-18RB.

In some embodiments, the signaling region may be derived from non-natural homodimers, such as interleukin-23 receptor (IL-23R) (see, e.g., Engelowski et al., 2018 [14], the contents of which is incorporated herein by reference in its entirety for all purposes), or interleukin-10 beta subunit (IL-10R2) (see, e.g., Mossner et al., 2020 [15], the contents of which is incorporated herein by reference in its entirety for all purposes).

In some embodiments, the signaling region may be derived from IL-6 beta chain (gp130) (see, e.g., Stuhlmann-Laeisz, et al., 2006 [16], the contents of which is incorporated herein by reference in its entirety for all purposes).

In some embodiments, the signaling region may be derived from natural Janus Kinase (JAK)/Signal Transducer and Activator of Transcription (STAT) homodimers including, but not limited to: Thrombopoietin Receptor (TpoR), Leptin Receptor (LEPR), Prolactin Receptor (PRLR), and Granulocyte Colony-Stimulating Factor Receptor (GCSFR) (see e.g., Staerk et al., 2011 [11], the contents of which is incorporated herein by reference in its entirety for all purposes).

In some embodiments, the signaling region may be derived from EpoR, or a portion or variant thereof. In further embodiments, the signaling region derived from EpoR does not comprise an Src homology region 2 domain-containing phosphatase-1 (SHP1) binding site.

In some embodiments, the signaling region derived from EpoR may comprise the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In some embodiments, the nucleotide sequence that encodes the signaling region derived from EpoR comprises the nucleotide sequence that encodes the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In some embodiments, the signaling region derived from EpoR may be encoded by the nucleotide sequence of SEQ ID NO: 45, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 45. In some embodiments, the signaling region derived from EpoR may comprise the amino acid sequence of SEQ ID NO: 46. In some embodiments, the nucleotide sequence that encodes the signaling region derived from EpoR comprises the nucleotide sequence set forth in SEQ ID NO: 45.

In some embodiments, the signaling region derived from EpoR may comprise the amino acid sequence of SEQ ID NO: 49, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 49. In some embodiments, the nucleotide sequence that encodes the signaling region derived from EpoR comprises the nucleotide sequence that encodes the amino acid sequence of SEQ ID NO: 49, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 49. In some embodiments, the signaling region derived from EpoR may be encoded by the nucleotide sequence of SEQ ID NO: 154, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 154. In some embodiments, the signaling region derived from EpoR may comprise the amino acid sequence of SEQ ID NO: 49. In some embodiments, the nucleotide sequence that encodes the signaling region derived from EpoR comprises the nucleotide sequence set forth in SEQ ID NO: 154.

In some embodiments, the signaling region may be derived from GHR, or a portion or variant thereof.

In some embodiments, the signaling region derived from GHR may comprise the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In some embodiments, the nucleotide sequence that encodes the signaling region derived from GHR comprises the nucleotide sequence that encodes the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In some embodiments, the signaling region derived from GHR may be encoded by the nucleotide sequence of SEQ ID NO: 43, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In some embodiments, the signaling region derived from GHR may comprise the amino acid sequence of SEQ ID NO: 44. In some embodiments, the nucleotide sequence that encodes the signaling region derived from EpoR comprises the nucleotide sequence set forth in SEQ ID NO: 43.

In some embodiments, the signaling region may be derived from a receptor tyrosine kinase, or a portion or a variant thereof. As an example, the receptor tyrosine kinase may be epidermal growth factor receptor (EGFR) (see, e.g., Kourouniotis et al., 2016 [12], the contents of which is incorporated herein by reference in its entirety for all purposes).

In some embodiments, the signaling region may be derived from a Toll-like receptor, or a portion or a variant thereof. In some embodiments, the Toll-like receptor may be: TLR1, TLR2, TLR3, TLR4, TLR5, TLR6, TLR7, TLR8, TLR9, or TLR10. In some embodiments, the Toll-like receptor may be a Toll-like receptor described in Zhang et al. 2002 [13], the contents of which is incorporated herein by reference in its entirety for all purposes.

In some embodiments, the signaling region may be derived from a signal transducing adaptor protein, or a portion or a variant thereof. In some embodiments, the signal transducing adaptor protein may be MyD88.

In some embodiments, the signaling region derived from MyD88 may comprise the amino acid sequence of SEQ ID NO: 38 or 220, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 38 or 220. In some embodiments, the nucleotide sequence that encodes the signaling region derived from MyD88 comprises the nucleotide sequence that encodes the amino acid sequence of SEQ ID NO: 38 or 220, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 38 or 220. In some embodiments, the signaling region derived from MyD88 may be encoded by the nucleotide sequence of SEQ ID NO: 37 or 219, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 37 or 219. In some embodiments, the signaling region derived from MyD88 may comprise the amino acid sequence of SEQ ID NO: 38 or 220. In some embodiments, the nucleotide sequence that encodes the signaling region derived from MyD88 comprises the nucleotide sequence set forth in SEQ ID NO: 37 or 219.

### Non-Limiting Examples of Chimeric Receptors

In some embodiments, the present disclosure provides a chimeric receptor which is a homodimer comprising two polypeptide monomers, wherein each polypeptide monomer may comprise:
1) an extracellular region comprising a homodimerizing leucine zipper motif;
2) a transmembrane region derived from EpoR, or a portion or variant thereof; and
3) an intracellular region comprising a signaling region derived from EpoR, or a portion or variant thereof and, optionally, a signaling region derived from MyD88, or a portion or variant thereof.

In some embodiments, the leucine zipper motif may be derived from transcription factor c-Jun. In some embodiments, the leucine zipper motif may comprise the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the nucleotide sequence that encodes the leucine zipper motif comprises the nucleotide sequence that encodes the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the leucine zipper motif is encoded by the nucleotide sequence of: SEQ ID NO: 3, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In some embodiments, the leucine zipper motif comprises the amino acid sequence set forth in SEQ ID NO: 9. In some embodiments, the nucleotide sequence that encodes the leucine zipper motif comprises the nucleotide sequence set forth in SEQ ID NO: 3.

In some embodiments, the transmembrane region derived from EpoR may comprise the amino acid sequence of SEQ ID NO: 48, SEQ ID NO: 104, SEQ ID NO: 128, SEQ ID NO: 106, SEQ ID NO: 130, SEQ ID NO: 108, or SEQ ID NO: 132, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48, SEQ ID NO: 104, SEQ ID NO: 128, SEQ ID NO: 106, SEQ ID NO: 130, SEQ ID NO: 108, or SEQ ID NO: 132. In some embodiments, the nucleotide sequence that encodes the transmembrane region derived from EpoR comprises the nucleotide sequence that encodes the amino acid sequence of SEQ ID NO: 48, SEQ ID NO: 104, SEQ ID NO: 128, SEQ ID NO: 106, SEQ ID NO: 130, or SEQ ID NO: 108, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48, SEQ ID NO: 104, SEQ ID NO: 128, SEQ ID NO: 106, SEQ ID NO: 130, or SEQ ID NO: 108, or SEQ ID NO: 132. In some embodiments, the transmembrane region derived from EpoR may be encoded by the nucleotide sequence of SEQ ID NO: 103, SEQ ID NO: 105, SEQ ID NO: 127, SEQ ID NO: 107, SEQ ID NO: 129, SEQ ID NO: 109, or SEQ ID NO: 131, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 103, SEQ ID NO: 105, SEQ ID NO: 127, SEQ ID NO: 107, SEQ ID NO: 129, SEQ ID NO: 109, or SEQ ID NO: 131. In some embodiments, the transmembrane region derived from EpoR comprises the amino acid sequence set forth in SEQ ID NO: 48, SEQ ID NO: 104, SEQ ID NO: 128, SEQ ID NO: 106, SEQ ID NO: 130, SEQ ID NO: 108, or SEQ ID NO: 132. In some embodiments, the nucleotide sequence that encodes the transmembrane region derived from EpoR comprises the nucleotide sequence set forth in SEQ ID NO: 103, SEQ ID NO: 105, SEQ ID NO: 127, SEQ ID NO: 107, SEQ ID NO: 129, SEQ ID NO: 109, or SEQ ID NO: 131.

In some embodiments, the signaling region derived from EpoR does not comprise the Src homology region 2 domain-containing phosphatase-1 (SHP1) binding site.

In some embodiments, the signaling region derived from EpoR may comprise the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In some embodiments, the nucleotide sequence that encodes the signaling region derived from EpoR comprises the nucleotide sequence that encodes the amino acid sequence of SEQ ID NO: 46, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In some embodiments, the signaling region derived from EpoR may be encoded by the nucleotide sequence of SEQ ID NO: 45, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 45. In some embodiments, the signaling region derived from EpoR may comprise the amino acid sequence of SEQ ID NO: 46. In some embodiments, the nucleotide sequence that encodes the signaling region derived from EpoR comprises the nucleotide sequence set forth in SEQ ID NO: 45.

In some embodiments, each polypeptide monomer of the chimeric receptor may comprise the amino acid sequence of SEQ ID NO: 112, 114, 116, or 118, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 112, SEQ ID NO: 114, SEQ ID NO: 116, or SEQ ID NO: 118. In some embodiments, the nucleotide sequence that encodes each polypeptide monomer comprises the nucleotide sequence that encodes the amino acid sequence of SEQ ID NO: 112, SEQ ID NO: 114, SEQ ID NO: 116, or SEQ ID NO: 118, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In some embodiments, each polypeptide monomer may be encoded by the nucleotide sequence of SEQ ID NO: 111, 113, 115, or 117, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 111, SEQ ID NO: 113, SEQ ID NO: 115, or SEQ ID NO: 117. In some embodiments, each polypeptide monomer of the chimeric receptor may comprise the amino acid sequence of SEQ ID NO: 112, 114, 116, or 118. In some embodiments, each polypeptide monomer may be encoded by the nucleotide sequence of SEQ ID NO: 111, 113, 115, or 117.

In some embodiments, the present disclosure provides a chimeric receptor which is a homodimer comprising two polypeptide monomers, wherein each polypeptide monomer may comprise:
1) an extracellular region comprising a homodimerizing leucine zipper motif;
2) a transmembrane region derived from GHR, or a portion or variant thereof, and
3) an intracellular region comprising a signaling region derived from GHR, or a portion or variant thereof and, optionally, a signaling region derived from MyD88, or a portion or variant thereof.

In some embodiments, the leucine zipper motif may be derived from transcription factor c-Jun. In some embodiments, the leucine zipper motif may comprise the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the nucleotide sequence that encodes the leucine zipper motif comprises the nucleotide sequence that encodes the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the leucine zipper motif is encoded by the nucleotide sequence of: SEQ ID NO: 3, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In some embodiments, the leucine zipper motif comprises the amino acid sequence set forth in SEQ ID NO: 9. In some embodiments, the nucleotide sequence that encodes the leucine zipper motif comprises the nucleotide sequence set forth in SEQ ID NO: 3.

In some embodiments, the transmembrane region derived from GHR may comprise the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52, SEQ ID NO: 134, SEQ ID NO: 136, SEQ ID NO: 138, SEQ ID NO: 140, SEQ ID NO: 142, or SEQ ID NO: 144. In some embodiments, the nucleotide sequence that encodes the transmembrane region derived from GHR comprises the nucleotide sequence that encodes the amino acid sequence of SEQ ID NO: 52, SEQ ID NO: 134, SEQ ID NO: 136, SEQ ID NO: 138, SEQ ID NO: 140, SEQ ID NO: 142, or SEQ ID NO: 144, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52, SEQ ID NO: 134, SEQ ID NO: 136, SEQ ID NO: 138, SEQ ID NO: 140, SEQ ID NO: 142, or SEQ ID NO: 144. In some embodiments, the transmembrane region derived from GHR may be encoded by the nucleotide sequence of SEQ ID NO: 133, SEQ ID NO: 135, SEQ ID NO: 137, SEQ ID NO: 139, SEQ ID NO: 141, SEQ ID NO: 143, SEQ ID NO: 145, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 133, SEQ ID NO: 135, SEQ ID NO: 137, SEQ ID NO: 139, SEQ ID NO: 141, SEQ ID NO: 143, or SEQ ID NO: 145. In some embodiments, the transmembrane region derived from GHR comprises the amino acid sequence set forth in SEQ ID NO: 52, SEQ ID NO: 134, SEQ ID NO: 136, SEQ ID NO: 138, SEQ ID NO: 140, SEQ ID NO: 142, or SEQ ID NO: 144. In some embodiments, the nucleotide sequence that encodes the transmembrane region derived from GHR comprises the nucleotide sequence set forth in SEQ ID NO: 133, SEQ ID NO: 135, SEQ ID NO: 137, SEQ ID NO: 139, SEQ ID NO: 141, SEQ ID NO: 143, or SEQ ID NO: 145.

In some embodiments, the signaling region derived from GHR may comprise the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In some embodiments, the nucleotide sequence that encodes the signaling region derived from GHR comprises the nucleotide sequence that encodes the amino acid sequence of SEQ ID NO: 44, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In some embodiments, the signaling region derived from GHR may be encoded by the nucleotide sequence of SEQ ID NO: 43, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In some embodiments, the signaling region derived from GHR may comprise the amino acid sequence of SEQ ID NO: 44. In some embodiments, the nucleotide sequence that encodes the signaling region derived from GHR comprises the nucleotide sequence set forth in SEQ ID NO: 43.

In some embodiments, the signaling region derived from MyD88 may comprise the amino acid sequence of SEQ ID NO: 220, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 220. In some embodiments, the nucleotide sequence that encodes the signaling region derived from MyD88 comprises the nucleotide sequence that encodes the amino acid sequence of SEQ ID NO: 220, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 220. In some embodiments, the signaling region derived from MyD88 may be encoded by the nucleotide sequence of SEQ ID NO: 219, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 219. In some embodiments, the signaling region derived from MyD88 may comprise the amino acid sequence of SEQ ID NO: 220. In some embodiments, the nucleotide sequence that encodes the signaling region derived from MyD88 comprises the nucleotide sequence set forth in SEQ ID NO: 219.

In some embodiments, each polypeptide monomer of the chimeric receptor may comprise the amino acid sequence of SEQ ID NO: 120, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 120. In some embodiments, the nucleotide sequence that encodes each polypeptide monomer comprises the nucleotide sequence that encodes the amino acid sequence of SEQ ID NO: 120, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 120. In some embodiments, each polypeptide monomer may be encoded by the nucleotide sequence of SEQ ID NO: 119, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 119. In some embodiments, each polypeptide monomer of the chimeric receptor may comprise the amino acid sequence of SEQ ID NO: 120. In some embodiments, each polypeptide monomer may be encoded by the nucleotide sequence of SEQ ID NO: 119.

In some embodiments, each polypeptide monomer of the chimeric receptor may comprise the amino acid sequence of SEQ ID NO: 221, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 221. In some embodiments, the nucleotide sequence that encodes each polypeptide monomer comprises the nucleotide sequence that encodes the amino acid sequence of SEQ ID NO: 221, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 221. In some embodiments, each polypeptide monomer may be encoded by the nucleotide sequence of SEQ ID NO: 222, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 222. In some embodiments, each polypeptide monomer of the chimeric receptor may comprise the amino acid sequence of SEQ ID NO: 221. In some embodiments, each polypeptide monomer may be encoded by the nucleotide sequence of SEQ ID NO: 222.

In some embodiments, each polypeptide monomer of the chimeric receptor may comprise amino acids 28-611 of the amino acid sequence of SEQ ID NO: 221, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with amino acids 28-611 of SEQ ID NO: 221. In some embodiments, the nucleotide sequence that encodes each polypeptide monomer comprises the nucleotide sequence that encodes amino acids 28-611 of the amino acid sequence of SEQ ID NO: 221, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with amino acids 28-611 of SEQ ID NO: 221. In some embodiments, each polypeptide monomer may be encoded by nucleotides 82-1833 of the nucleotide sequence of SEQ ID NO: 222, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with nucleotides 82-1833 of SEQ ID NO: 222. In some embodiments, each polypeptide monomer of the chimeric receptor may comprise amino acids 28-611 of the amino acid sequence of SEQ ID NO: 221. In some embodiments, each polypeptide monomer may be encoded by nucleotides 82-1833 of the nucleotide sequence of SEQ ID NO: 222.

In some embodiments, the present disclosure provides a chimeric receptor which is a homodimer comprising two polypeptide monomers, wherein each polypeptide monomer may comprise:
1) an extracellular region comprising a homodimerizing leucine zipper motif;
2) a transmembrane region derived from IL-18Ra, IL-18RB or CD28, or a portion or variant thereof; and
3) an intracellular region comprising a signaling region derived from MyD88, or a portion or variant thereof.

In some embodiments, the leucine zipper motif may be derived from transcription factor c-Jun. In some embodiments, the leucine zipper motif may comprise the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the nucleotide sequence that encodes the leucine zipper motif comprises the nucleotide sequence that encodes the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In some embodiments, the leucine zipper motif is encoded by the nucleotide sequence of: SEQ ID NO: 3, or a sequence having at least 50%, at least 55%, at least 60%, at least 65%, at least 89%, at least 90%, at least 91%, at least 92%, at least 93%, at least 94%, at least 95%, at least 96%, at least 97%, at least 98%, or at least 99%, sequence identity with SEQ ID NO: 3. In some embodiments, the leucine zipper motif comprises the amino acid sequence set forth in SEQ ID NO: 9. In some embodiments, the nucleotide sequence that encodes the leucine zipper motif comprises the nucleotide sequence set forth in SEQ ID NO: 3.

In some embodiments, the transmembrane region may comprise the amino acid sequence of SEQ ID NO: 40, SEQ ID NO: 36, or SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40, SEQ ID NO: 36, or SEQ ID NO: 42. In some embodiments, the nucleotide sequence that encodes the transmembrane region comprises the nucleotide sequence that encodes the amino acid sequence of SEQ ID NO: 40, SEQ ID NO: 36, or SEQ ID NO: 42, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 40, SEQ ID NO: 36, or SEQ ID NO: 42. In some embodiments, the transmembrane region may be encoded by the nucleotide sequence of SEQ ID NO: 39, SEQ ID NO: 35, or SEQ ID NO: 41, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 39, SEQ ID NO: 35, or SEQ ID NO: 41. In some embodiments, the transmembrane region comprises the amino acid sequence set forth in SEQ ID NO: 40, SEQ ID NO: 36, or SEQ ID NO: 42. In some embodiments, the nucleotide sequence that encodes the transmembrane region comprises the nucleotide sequence set forth in SEQ ID NO: 39, SEQ ID NO: 35, or SEQ ID NO: 41.

In some embodiments, the signaling region derived from MyD88 may comprise the amino acid sequence of SEQ ID NO: 38 or 220, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 38 or 220. In some embodiments, the nucleotide sequence that encodes the signaling region derived from MyD88 comprises the nucleotide sequence that encodes the amino acid sequence of SEQ ID NO: 38, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 38 or 220. In some embodiments, the signaling region derived from MyD88 may be encoded by the nucleotide sequence of SEQ ID NO: 37 or 219, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 37 or 219. In some embodiments, the signaling region derived from MyD88 may comprise the amino acid sequence of SEQ ID NO: 38 or 220. In some embodiments, the nucleotide sequence that encodes the signaling region derived from MyD88 comprises the nucleotide sequence set forth in SEQ ID NO: 37 or 219.

In some embodiments, each polypeptide monomer of the chimeric receptor may comprise the amino acid sequence of SEQ ID NO: 122, 124, or 126, or a sequence thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122, SEQ ID NO: 124, or SEQ ID NO: 126. In some embodiments, the nucleotide sequence that encodes each polypeptide monomer comprises the nucleotide sequence that encodes the amino acid sequence of SEQ ID NO: 122, SEQ ID NO: 124, or SEQ ID NO: 126, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122, SEQ ID NO: 124, or SEQ ID NO: 126. In some embodiments, each polypeptide monomer may be encoded by the nucleotide sequence of SEQ ID NO: 121, 123, or 125, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 121, SEQ ID NO: 123, or SEQ ID NO: 125. In some embodiments, each polypeptide monomer of the chimeric receptor may comprise the amino acid sequence of SEQ ID NO: 122, SEQ ID NO: 124, or SEQ ID NO: 126. In some embodiments, each polypeptide monomer may be encoded by the nucleotide sequence of SEQ ID NO: 121, SEQ ID NO: 123, or SEQ ID NO: 125.

In some embodiments, the chimeric receptor may comprise a homodimer comprising two polypeptide monomers, wherein each polypeptide monomer may comprise the amino acid sequence set forth in SEQ ID NO: 112, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 112. In some embodiments, each polypeptide monomer may comprise an amino acid sequence that is encoded by the nucleic acid sequence SEQ ID NO: 111, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 111.

In some embodiments, the chimeric receptor may comprise a homodimer comprising two polypeptide monomers, wherein each polypeptide monomer may comprise the amino acid sequence set forth in SEQ ID NO: 114, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 114. In some embodiments, each polypeptide monomer may comprise an amino acid sequence that is encoded by the nucleic acid sequence SEQ ID NO: 113, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 113.

In some embodiments, the chimeric receptor may comprise a homodimer comprising two polypeptide monomers, wherein each polypeptide monomer may comprise the amino acid sequence set forth in SEQ ID NO: 116, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 116. In some embodiments, each polypeptide monomer may comprise an amino acid sequence that is encoded by the nucleic acid sequence SEQ ID NO: 115, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 115.

In some embodiments, the chimeric receptor may comprise a homodimer comprising two polypeptide monomers, wherein each polypeptide monomer may comprise the amino acid sequence set forth in SEQ ID NO: 118, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 118. In some embodiments, each polypeptide monomer may comprise an amino acid sequence that is encoded by the nucleic acid sequence SEQ ID NO: 117, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 117.

In some embodiments, the chimeric receptor may comprise a homodimer comprising two polypeptide monomers, wherein each polypeptide monomer may comprise the amino acid sequence set forth in SEQ ID NO: 120, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 120. In some embodiments, each polypeptide monomer may comprise an amino acid sequence that is encoded by the nucleic acid sequence SEQ ID NO: 119, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 119.

In some embodiments, the chimeric receptor may comprise a homodimer comprising two polypeptide monomers, wherein each polypeptide monomer may comprise the amino acid sequence set forth in SEQ ID NO: 122, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 122. In some embodiments, each polypeptide monomer may comprise an amino acid sequence that is encoded by the nucleic acid sequence SEQ ID NO: 121, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 121.

In some embodiments, the chimeric receptor may comprise a homodimer comprising two polypeptide monomers, wherein each polypeptide monomer may comprise the amino acid sequence set forth in SEQ ID NO: 124, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 124. In some embodiments, each polypeptide monomer may comprise an amino acid sequence that is encoded by the nucleic acid sequence SEQ ID NO: 123, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 123.

In some embodiments, the chimeric receptor may comprise a homodimer comprising two polypeptide monomers, wherein each polypeptide monomer may comprise the amino acid sequence set forth in SEQ ID NO: 126, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 126. In some embodiments, each polypeptide monomer may comprise an amino acid sequence that is encoded by the nucleic acid sequence SEQ ID NO: 125, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 125.

In some embodiments, the chimeric receptor may comprise a homodimer comprising two polypeptide monomers, wherein each polypeptide monomer may comprise the amino acid sequence set forth in SEQ ID NO: 146, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 146. In some embodiments, each polypeptide monomer may comprise an amino acid sequence that is encoded by the nucleic acid sequence SEQ ID NO: 147, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 147.

In some embodiments, the chimeric receptor may comprise a homodimer comprising two polypeptide monomers, wherein each polypeptide monomer may comprise the amino acid sequence set forth in SEQ ID NO: 148, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 148. In some embodiments, each polypeptide monomer may comprise an amino acid sequence that is encoded by the nucleic acid sequence SEQ ID NO: 149, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 149.

In some embodiments, the chimeric receptor may comprise a homodimer comprising two polypeptide monomers, wherein each polypeptide monomer may comprise the amino acid sequence set forth in SEQ ID NO: 221, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 221. In some embodiments, each polypeptide monomer may comprise an amino acid sequence that is encoded by the nucleic acid sequence SEQ ID NO: 222, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 222.

In some embodiments, the chimeric receptor may comprise a homodimer comprising two polypeptide monomers, wherein each polypeptide monomer may comprise amino acids 28-611 of the amino acid sequence set forth in SEQ ID NO: 221, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with amino acids 28-611 of SEQ ID NO: 221. In some embodiments, each polypeptide monomer may comprise an amino acid sequence that is encoded by nucleotides 82-1833 of the nucleic acid sequence SEQ ID NO: 222, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with nucleotides 82-1833 of SEQ ID NO: 222.

In some embodiments, the chimeric receptor may comprise a homodimer comprising two polypeptide monomers, wherein each polypeptide monomer comprises: 1) an extracellular region comprising at least one homodimerizing motif; 2) a transmembrane region; 3) an intracellular region comprising at least one signaling region derived from a cell-surface receptor or a signal transducing adaptor protein, or a portion or a variant thereof; and, optionally, 4) at least one additional gene.

Table 2 shows non-limiting exemplary constructs which may comprise: 1) an extracellular region comprising at least one homodimerizing motif; 2) a transmembrane region; 3) an intracellular region comprising at least one signaling region derived from a cell-surface receptor or a signal transducing adaptor protein, or a portion or a variant thereof; and, optionally, 4) at least one additional gene.

TABLE 2
Exemplary Polypeptide Component Combinations
ZipEpoR: cJunzip.hEpoRmut.2A.mClover
Nucleotide Sequence:
Leader:
ATGGATTGGATCTGGCGGATCCTGTTCCTTGTGGGAGCTGCCACAGGCGCCCACAGC (SEQ ID NO: 1)
FLAG:
GACTACAAGGACGACGACGACAAG (SEQ ID NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
hEpoRmut:
CTGATCCTGACACTGAGCCTGATCCTGGTGGTCATCCTGGTGCTGCTGACAGTGCTGGCCCTGCTGTCTCATAGA
AGGGCCCTGAAGCAGAAGATCTGGCCTGGCATCCCATCTCCAGAGAGCGAGTTCGAGGGCCTGTTCACCACACAC
AAGGGCAACTTCCAGCTGTGGCTGTACCAGAACGATGGCTGCCTTTGGTGGTCCCCTTGCACACCCTTTACCGAG
GATCCACCAGCCAGCCTGGAAGTGCTGAGCGAGAGATGTTGGGGCACAATGCAGGCCGTGGAACCCGGCACAGAT
GATGAAGGACCTCTGCTGGAACCTGTGGGCTCTGAACATGCCCAGGACACCTATCTGGTGCTGGACAAGTGGCTG
CTGCCCCGGAATCCTCCATCTGAGGATTTGCCTGGACCTGGCGGCTCCGTGGATATCGTGGCTATGGATGAGGGC
AGCGAGGCCAGCTCTTGTTCTTCTGCCCTGGCCAGCAAGCCTTCTCCAGAAGGCGCTTCTGCCGCCAGCTTCGAG
TACACCATTCTGGACCCTAGCAGCCAGCTGCTGAGGCCT (SEQ ID NO: 4)
P2A:
GGCAGCGGCGCCACCAACTTTAGCCTGCTGAAACAAGCCGGCGACGTGGAAGAGAATCCCGGACCT (SEQ ID
NO: 5)
mClover3:
ATGGTGAGCAAGGGCGAGGAGCTGTTCACCGGGGTGGTGCCCATCCTGGTCGAGCTGGACGGCGACGTAAACGGC
CACAAGTTCAGCGTCCGCGGCGAaGGtGAGGGaGATGCCACCAACGGCAAGCTGACCCTGAAGTTCATCTGCACC
ACCGGCAAGCTGCCCGTGCCCTGGCCCACCCTCGTGACCACCTTCGGCTACGGCGTGGCCTGCTTCAGCCGCTAC
CCCGACCACATGAAGCAGCACGACTTCTTCAAGTCCGCCATGCCCGAAGGCTACGTCCAGGAGCGCACCATCTCT
TTCAAGGACGACGGTACCTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTTCAACAGCCACTAC
GTCTATATCACGGCCGACAAGCAGAAGAACTGCATCAAGGCTAACTTCAAGATCCGCCACAACGTTGAGGACGGC
AGCGTGCAGCTCGCCGACCACTACCAGCAGAACACCCCCATCGGCGACGGCCCCGTGCTGCTGCCCGACAACCAC
TACCTGAGCCATCAGTCCAAGCTGAGCAAAGACCCCAACGAGAAGCGCGATCACATGGTCCTGCTGGAGTTCGTG
ACCGCCGCCGGGATTACACATGGCATGGACGAGCTGTACAAGTAA (SEQ ID NO: 6)
Amino acid Sequence:
Leader:
MDWIWRILFLVGAATGAHS (SEQ ID NO: 7)
FLAG:
DYKDDDDK (SEQ ID NO: 8)
cJun:
IARLEEKVKTLKAQNSELASTANMLREQVAQLKQKVMNH (SEQ ID NO: 9)
hEpoRmut:
LILTLSLILVVILVLLTVLALLSHRRALKQKIWPGIPSPESE FEGLFTTHKGNFQLWLYQNDGCLWWSPCTPFTE
DPPASLEVLSERCWGTMQAVEPGTDDEGPLLEPVGSEHAQDTYLVLDKWLLPRNPPSEDLPGPGGSVDIVAMDEG
SEASSCSSALASKPSPEGASAASFEYTILDPSSQLLRP (SEQ ID NO: 10)
P2A:
GSGATNFSLLKQAGDVEENPGP (SEQ ID NO: 11)
mClover3:
MVSKGEELFTGVVPILVELDGDVNGHKFSVRGEGEGDATNGKLTLKFICTTGKLPVPWPTLVTTEGYGVACESRY
PDHMKQHDFFKSAMPEGYVQERTISFKDDGTYKTRAEVKFEGDTLVNRIELKGIDFKEDGNILGHKLEYNENSHY
VYITADKQKNCIKANFKIRHNVEDGSVQLADHYQQNTPIGDGPVLLPDNHYLSHQSKLSKDPNEKRDHMVLLEFV
TAAGITHGMDELYK (SEQ ID NO: 12)
ZipEpoR.del1: cJunzip.hEpoRmut.del1.2A.mClover
Nucleotide Sequence:
Leader:
ATGGATTGGATCTGGCGGATCCTGTTCCTTGTGGGAGCTGCCACAGGCGCCCACAGC (SEQ ID NO: 1)
FLAG:
GACTACAAGGACGACGACGACAAG (SEQ ID NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
hEpoRmut:
ATCCTGACACTGAGCCTGATCCTGGTGGTCATCCTGGTGCTGCTGACAGTGCTGGCCCTGCTGTCTCATAGAAGG
GCCCTGAAGCAGAAGATCTGGCCTGGCATCCCATCTCCAGAGAGCGAGTTCGAGGGCCTGTTCACCACACACAAG
GGCAACTTCCAGCTGTGGCTGTACCAGAACGATGGCTGCCTTTGGTGGTCCCCTTGCACACCCTTTACCGAGGAT
CCACCAGCCAGCCTGGAAGTGCTGAGCGAGAGATGTTGGGGCACAATGCAGGCCGTGGAACCCGGCACAGATGAT
GAAGGACCTCTGCTGGAACCTGTGGGCTCTGAACATGCCCAGGACACCTATCTGGTGCTGGACAAGTGGCTGCTG
CCCCGGAATCCTCCATCTGAGGATTTGCCTGGACCTGGCGGCTCCGTGGATATCGTGGCTATGGATGAGGGCAGC
GAGGCCAGCTCTTGTTCTTCTGCCCTGGCCAGCAAGCCTTCTCCAGAAGGCGCTTCTGCCGCCAGCTTCGAGTAC
ACCATTCTGGACCCTAGCAGCCAGCTGCTGAGGCCT (SEQ ID NO: 13)
P2A:
GGCAGCGGCGCCACCAACTTTAGCCTGCTGAAACAAGCCGGCGACGTGGAAGAGAATCCCGGACCT (SEQ ID
NO: 5)
mClover3:
ATGGTGAGCAAGGGCGAGGAGCTGTTCACCGGGGTGGTGCCCATCCTGGTCGAGCTGGACGGCGACGTAAACGGC
CACAAGTTCAGCGTCCGCGGCGAaGGtGAGGGaGATGCCACCAACGGCAAGCTGACCCTGAAGTTCATCTGCACC
ACCGGCAAGCTGCCCGTGCCCTGGCCCACCCTCGTGACCACCTTCGGCTACGGCGTGGCCTGCTTCAGCCGCTAC
CCCGACCACATGAAGCAGCACGACTTCTTCAAGTCCGCCATGCCCGAAGGCTACGTCCAGGAGCGCACCATCTCT
TTCAAGGACGACGGTACCTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTTCAACAGCCACTAC
GTCTATATCACGGCCGACAAGCAGAAGAACTGCATCAAGGCTAACTTCAAGATCCGCCACAACGTTGAGGACGGC
AGCGTGCAGCTCGCCGACCACTACCAGCAGAACACCCCCATCGGCGACGGCCCCGTGCTGCTGCCCGACAACCAC
TACCTGAGCCATCAGTCCAAGCTGAGCAAAGACCCCAACGAGAAGCGCGATCACATGGTCCTGCTGGAGTTCGTG
ACCGCCGCCGGGATTACACATGGCATGGACGAGCTGTACAAGTAA (SEQ ID NO: 6)
Amino acid Sequence:
Leader:
MDWIWRILFLVGAATGAHS (SEQ ID NO: 7)
FLAG:
DYKDDDDK (SEQ ID NO: 8)
cJun:
IARLEEKVKTLKAQNSELASTANMLREQVAQLKQKVMNH (SEQ ID NO: 9)
hEpoRmut:
ILTLSLILVVILVLLTVLALLSHRRALKQKIWPGIPSPESEFEGLFTTHKGNFQLWLYQNDGCLWWSPCTPFTED
PPASLEVLSERCWGTMQAVEPGTDDEGPLLEPVGSEHAQDTYLVLDKWLLPRNPPSEDLPGPGGSVDIVAMDEGS
EASSCSSALASKPSPEGASAASFEYTILDPSSQLLRP (SEQ ID NO: 14)
P2A:
GSGATNFSLLKQAGDVEENPGP (SEQ ID NO: 11)
mClover3:
MVSKGEELFTGVVPILVELDGDVNGHKFSVRGEGEGDATNGKLTLKFICTTGKLPVPWPTLVTTFGYGVACFSRY
PDHMKQHDFFKSAMPEGYVQERTISFKDDGTYKTRAEVKFEGDTLVNRIELKGIDFKEDGNILGHKLEYNENSHY
VYITADKQKNCIKANFKIRHNVEDGSVQLADHYQQNTPIGDGPVLLPDNHYLSHQSKLSKDPNEKRDHMVLLEFV
TAAGITHGMDELYK (SEQ ID NO: 12)
ZipEpoR.del3: cJunzip.hEpoRmut.del3.2A.mClover
Nucleotide Sequence:
Leader:
ATGGATTGGATCTGGCGGATCCTGTTCCTTGTGGGAGCTGCCACAGGCGCCCACAGC (SEQ ID NO: 1)
FLAG:
GACTACAAGGACGACGACGACAAG (SEQ ID NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
hEpoRmut:
ACACTGAGCCTGATCCTGGTGGTCATCCTGGTGCTGCTGACAGTGCTGGCCCTGCTGTCTCATAGAAGGGCCCTG
AAGCAGAAGATCTGGCCTGGCATCCCATCTCCAGAGAGCGAGTTCGAGGGCCTGTTCACCACACACAAGGGCAAC
TTCCAGCTGTGGCTGTACCAGAACGATGGCTGCCTTTGGTGGTCCCCTTGCACACCCTTTACCGAGGATCCACCA
GCCAGCCTGGAAGTGCTGAGCGAGAGATGTTGGGGCACAATGCAGGCCGTGGAACCCGGCACAGATGATGAAGGA
CCTCTGCTGGAACCTGTGGGCTCTGAACATGCCCAGGACACCTATCTGGTGCTGGACAAGTGGCTGCTGCCCCGG
AATCCTCCATCTGAGGATTTGCCTGGACCTGGCGGCTCCGTGGATATCGTGGCTATGGATGAGGGCAGCGAGGCC
AGCTCTTGTTCTTCTGCCCTGGCCAGCAAGCCTTCTCCAGAAGGCGCTTCTGCCGCCAGCTTCGAGTACACCATT
CTGGACCCTAGCAGCCAGCTGCTGAGGCCT (SEQ ID NO: 15)
P2A:
GGCAGCGGCGCCACCAACTTTAGCCTGCTGAAACAAGCCGGCGACGTGGAAGAGAATCCCGGACCT (SEQ ID
NO: 5)
mClover3:
ATGGTGAGCAAGGGCGAGGAGCTGTTCACCGGGGTGGTGCCCATCCTGGTCGAGCTGGACGGCGACGTAAACGGC
CACAAGTTCAGCGTCCGCGGCGAaGGtGAGGGaGATGCCACCAACGGCAAGCTGACCCTGAAGTTCATCTGCACC
ACCGGCAAGCTGCCCGTGCCCTGGCCCACCCTCGTGACCACCTTCGGCTACGGCGTGGCCTGCTTCAGCCGCTAC
CCCGACCACATGAAGCAGCACGACTTCTTCAAGTCCGCCATGCCCGAAGGCTACGTCCAGGAGCGCACCATCTCT
TTCAAGGACGACGGTACCTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTTCAACAGCCACTAC
GTCTATATCACGGCCGACAAGCAGAAGAACTGCATCAAGGCTAACTTCAAGATCCGCCACAACGTTGAGGACGGC
AGCGTGCAGCTCGCCGACCACTACCAGCAGAACACCCCCATCGGCGACGGCCCCGTGCTGCTGCCCGACAACCAC
TACCTGAGCCATCAGTCCAAGCTGAGCAAAGACCCCAACGAGAAGCGCGATCACATGGTCCTGCTGGAGTTCGTG
ACCGCCGCCGGGATTACACATGGCATGGACGAGCTGTACAAGTAA (SEQ ID NO: 6)
Amino acid Sequence:
Leader:
MDWIWRILFLVGAATGAHS (SEQ ID NO: 7)
FLAG:
DYKDDDDK (SEQ ID NO: 8)
cJun:
IARLEEKVKTLKAQNSELASTANMLREQVAQLKQKVMNH (SEQ ID NO: 9)
hEpoRmut:
TLSLILVVILVLLTVLALLSHRRALKQKIWPGIPSPESEFEGLFTTHKGNFQLWLYQNDGCLWWSPCTPFTEDPP
ASLEVLSERCWGTMQAVEPGTDDEGPLLEPVGSEHAQDTYLVLDKWLLPRNPPSEDLPGPGGSVDIVAMDEGSEA
SSCSSALASKPSPEGASAASFEYTILDPSSQLLRP (SEQ ID NO: 16)
P2A:
GSGATNFSLLKQAGDVEENPGP (SEQ ID NO: 11)
mClover3:
MVSKGEELFTGVVPILVELDGDVNGHKFSVRGEGEGDATNGKLTLKFICTTGKLPVPWPTLVTTFGYGVACESRY
PDHMKQHDFFKSAMPEGYVQERTISFKDDGTYKTRAEVKFEGDTLVNRIELKGIDFKEDGNILGHKLEYNENSHY
VYITADKQKNCIKANFKIRHNVEDGSVQLADHYQQNTPIGDGPVLLPDNHYLSHQSKLSKDPNEKRDHMVLLEFV
TAAGITHGMDELYK (SEQ ID NO: 12)
ZipEpoR.del5: cJunzip.hEpoRmut.del5.2A.mClover
Nucleotide Sequence:
Leader:
ATGGATTGGATCTGGCGGATCCTGTTCCTTGTGGGAGCTGCCACAGGCGCCCACAGC (SEQ NO: 1)
FLAG:
GACTACAAGGACGACGACGACAAG (SEQ NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
hEpoRmut:
AGCCTGATCCTGGTGGTCATCCTGGTGCTGCTGACAGTGCTGGCCCTGCTGTCTCATAGAAGGGCCCTGAAGCAG
AAGATCTGGCCTGGCATCCCATCTCCAGAGAGCGAGTTCGAGGGCCTGTTCACCACACACAAGGGCAACTTCCAG
CTGTGGCTGTACCAGAACGATGGCTGCCTTTGGTGGTCCCCTTGCACACCCTTTACCGAGGATCCACCAGCCAGC
CTGGAAGTGCTGAGCGAGAGATGTTGGGGCACAATGCAGGCCGTGGAACCCGGCACAGATGATGAAGGACCTCTG
CTGGAACCTGTGGGCTCTGAACATGCCCAGGACACCTATCTGGTGCTGGACAAGTGGCTGCTGCCCCGGAATCCT
CCATCTGAGGATTTGCCTGGACCTGGCGGCTCCGTGGATATCGTGGCTATGGATGAGGGCAGCGAGGCCAGCTCT
TGTTCTTCTGCCCTGGCCAGCAAGCCTTCTCCAGAAGGCGCTTCTGCCGCCAGCTTCGAGTACACCATTCTGGAC
CCTAGCAGCCAGCTGCTGAGGCCT (SEQ ID NO: 17)
P2A:
GGCAGCGGCGCCACCAACTTTAGCCTGCTGAAACAAGCCGGCGACGTGGAAGAGAATCCCGGACCT (SEQ ID
NO: 5)
mClover3:
ATGGTGAGCAAGGGCGAGGAGCTGTTCACCGGGGTGGTGCCCATCCTGGTCGAGCTGGACGGCGACGTAAACGGC
CACAAGTTCAGCGTCCGCGGCGAaGGtGAGGGaGATGCCACCAACGGCAAGCTGACCCTGAAGTTCATCTGCACC
ACCGGCAAGCTGCCCGTGCCCTGGCCCACCCTCGTGACCACCTTCGGCTACGGCGTGGCCTGCTTCAGCCGCTAC
CCCGACCACATGAAGCAGCACGACTTCTTCAAGTCCGCCATGCCCGAAGGCTACGTCCAGGAGCGCACCATCTCT
TTCAAGGACGACGGTACCTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTTCAACAGCCACTAC
GTCTATATCACGGCCGACAAGCAGAAGAACTGCATCAAGGCTAACTTCAAGATCCGCCACAACGTTGAGGACGGC
AGCGTGCAGCTCGCCGACCACTACCAGCAGAACACCCCCATCGGCGACGGCCCCGTGCTGCTGCCCGACAACCAC
TACCTGAGCCATCAGTCCAAGCTGAGCAAAGACCCCAACGAGAAGCGCGATCACATGGTCCTGCTGGAGTTCGTG
ACCGCCGCCGGGATTACACATGGCATGGACGAGCTGTACAAGTAA (SEQ ID NO: 6)
Amino acid Sequence:
Leader:
MDWIWRILFLVGAATGAHS (SEQ ID NO: 7)
FLAG:
DYKDDDDK (SEQ ID NO: 8)
cJun:
IARLEEKVKTLKAQNSELASTANMLREQVAQLKQKVMNH (SEQ ID NO: 9)
hEpoRmut:
SLILVVILVLLTVLALLSHRRALKQKIWPGIPSPESEFEGLFTTHKGNFQLWLYQNDGCLWWSPCTPFTEDPPAS
LEVLSERCWGTMQAVEPGTDDEGPLLEPVGSEHAQDTYLVLDKWLLPRNPPSEDLPGPGGSVDIVAMDEGSEASS
CSSALASKPSPEGASAASFEYTILDPSSQLLRP (SEQ ID NO: 18)
P2A:
GSGATNFSLLKQAGDVEENPGP (SEQ ID NO: 11)
mClover3:
MVSKGEELFTGVVPILVELDGDVNGHKFSVRGEGEGDATNGKLTLKFICTTGKLPVPWPTLVTTFGYGVACESRY
PDHMKQHDFFKSAMPEGYVQERTISFKDDGTYKTRAEVKFEGDTLVNRIELKGIDFKEDGNILGHKLEYNENSHY
VYITADKQKNCIKANFKIRHNVEDGSVQLADHYQQNTPIGDGPVLLPDNHYLSHQSKLSKDPNEKRDHMVLLEFV
TAAGITHGMDELYK (SEQ ID NO: 12)
ZipGHR: cJunzip.hGHR.2A.mClover
Nucleotide Sequence:
Leader:
ATGGATTGGATCTGGCGGATCCTGTTCCTTGTGGGAGCTGCCACAGGCGCCCACAGC (SEQ ID NO: 1)
FLAG:
GACTACAAGGACGACGACGACAAG (SEQ ID NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
hGHR:
TTCCCCTGGCTGCTGATCATCATCTTCGGCATCTTTGGCCTGACCGTGATGCTGTTCGTGTTCCTGTTCAGCAAG
CAGCAGCGGATCAAGATGCTGATCCTGCCTCCTGTGCCTGTGCCTAAGATCAAGGGCATCGACCCCGACCTGCTG
AAAGAGGGCAAGCTGGAAGAAGTGAACACCATCCTGGCCATCCACGACAGCTACAAGCCCGAGTTCCACAGCGAC
GATAGCTGGGTCGAGTTCATCGAGCTGGACATCGACGAGCCCGACGAGAAAACCGAGGAAAGCGACACCGACAGA
CTGCTGAGCAGCGACCACGAGAAGTCCCACTCTAACCTGGGCGTGAAGGATGGCGATAGCGGCAGAACAAGCTGC
TGCGAGCCCGATATCCTGGAAACCGACTTCAACGCCAACGACATCCACGAGGGCACCAGCGAAGTTGCCCAGCCT
CAAAGACTGAAGGGCGAAGCCGATCTGCTGTGCCTGGACCAGAAGAACCAGAACAACAGCCCCTACCACGACGCC
TGTCCTGCTACACAGCAGCCTAGTGTGATCCAGGCCGAGAAGAACAAGCCCCAGCCTCTGCCTACAGAGGGCGCC
GAATCTACACATCAGGCCGCTCACATCCAGCTGAGCAACCCTAGCAGCCTGAGCAACATCGACTTCTACGCCCAA
GTGTCCGACATCACACCAGCCGGATCTGTGGTGCTGTCTCCCGGCCAGAAAAACAAGGCCGGCATGTCCCAGTGC
GACATGCACCCTGAAATGGTGTCCCTGTGCCAAGAGAACTTCCTGATGGACAACGCCTACTTCTGCGAGGCCGAC
GCCAAGAAATGCATCCCTGTGGCTCCCCACATCAAGGTGGAAAGCCACATTCAGCCCAGCCTGAATCAAGAGGAT
ATCTACATCACCACCGAGAGCCTGACCACCGCTGCTGGTAGACCTGGAACAGGCGAGCATGTGCCTGGCTCTGAA
ATGCCCGTGCCTGACTACACCAGCATCCACATCGTGCAGAGCCCTCAGGGCCTGATCCTGAATGCTACAGCTCTG
CCCCTGCCAGACAAAGAGTTCCTGTCTAGCTGCGGCTACGTGTCCACCGACCAGCTGAACAAGATCATGCCC
(SEQ ID NO: 19)
P2A:
GGCAGCGGCGCCACCAACTTTAGCCTGCTGAAACAAGCCGGCGACGTGGAAGAGAATCCCGGACCT (SEQ ID
NO: 5)
mClover3:
ATGGTGAGCAAGGGCGAGGAGCTGTTCACCGGGGTGGTGCCCATCCTGGTCGAGCTGGACGGCGACGTAAACGGC
CACAAGTTCAGCGTCCGCGGCGAaGGtGAGGGaGATGCCACCAACGGCAAGCTGACCCTGAAGTTCATCTGCACC
ACCGGCAAGCTGCCCGTGCCCTGGCCCACCCTCGTGACCACCTTCGGCTACGGCGTGGCCTGCTTCAGCCGCTAC
CCCGACCACATGAAGCAGCACGACTTCTTCAAGTCCGCCATGCCCGAAGGCTACGTCCAGGAGCGCACCATCTCT
TTCAAGGACGACGGTACCTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTTCAACAGCCACTAC
GTCTATATCACGGCCGACAAGCAGAAGAACTGCATCAAGGCTAACTTCAAGATCCGCCACAACGTTGAGGACGGC
AGCGTGCAGCTCGCCGACCACTACCAGCAGAACACCCCCATCGGCGACGGCCCCGTGCTGCTGCCCGACAACCAC
TACCTGAGCCATCAGTCCAAGCTGAGCAAAGACCCCAACGAGAAGCGCGATCACATGGTCCTGCTGGAGTTCGTG
ACCGCCGCCGGGATTACACATGGCATGGACGAGCTGTACAAGTAA (SEQ ID NO: 6)
Amino acid Sequence:
Leader:
MDWIWRILFLVGAATGAHS (SEQ ID NO: 7)
FLAG:
DYKDDDDK (SEQ ID NO: 8)
cJun:
IARLEEKVKTLKAQNSELASTANMLREQVAQLKQKVMNH (SEQ ID NO: 9)
hGHR:
FPWLLIIIFGIFGLTVMLFVFLESKQQRIKMLILPPVPVPKIKGIDPDLLKEGKLEEVNTILAIHDSYKPEFHSD
DSWVEFIELDIDEPDEKTEESDTDRLLSSDHEKSHSNLGVKDGDSGRTSCCEPDILETDENANDIHEGTSEVAQP
QRLKGEADLLCLDQKNQNNSPYHDACPATQQPSVIQAEKNKPQPLPTEGAESTHQAAHIQLSNPSSLSNIDEYAQ
VSDITPAGSVVLSPGQKNKAGMSQCDMHPEMVSLCQENFLMDNAYFCEADAKKCIPVAPHIKVESHIQPSLNQED
IYITTESLTTAAGRPGTGEHVPGSEMPVPDYTSIHIVQSPQGLILNATALPLPDKEFLSSCGYVSTDQLNKIMP
(SEQ ID NO: 20)
P2A:
GSGATNFSLLKQAGDVEENPGP (SEQ ID NO: 11)
mClover3:
MVSKGEELFTGVVPILVELDGDVNGHKFSVRGEGEGDATNGKLTLKFICTTGKLPVPWPTLVTTFGYGVACFSRY
PDHMKQHDFFKSAMPEGYVQERTISFKDDGTYKTRAEVKFEGDTLVNRIELKGIDFKEDGNILGHKLEYNENSHY
VYITADKQKNCIKANFKIRHNVEDGSVQLADHYQQNTPIGDGPVLLPDNHYLSHQSKLSKDPNEKRDHMVLLEFV
TAAGITHGMDELYK (SEQ ID NO: 12)
Jun IL2Rb
Nucleotide Sequence:
Leader:
ATGGATTGGATCTGGCGGATCCTGTTCCTTGTGGGAGCTGCCACAGGCGCCCACAGC (SEQ ID NO: 1)
FLAG:
GACTACAAGGACGACGACGACAAG (SEQ ID NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
hIL2Rb:
ATCCCCTGGCTGGGACATCTGCTCGTTGGACTGTCTGGCGCCTTCGGCTTCATCATCCTGGTGTACCTGCTGATC
AACTGCCGGAACACAGGCCCTTGGCTGAAGAAAGTGCTGAAGTGCAACACCCCTGATCCGAGCAAGTTCTTTAGC
CAGCTGAGCAGCGAGCATGGCGGCGACGTTCAGAAATGGCTGTCTAGCCCATTTCCTAGCAGCAGCTTCAGCCCT
GGTGGACTGGCCCCTGAGATTAGCCCTCTGGAAGTGCTGGAACGGGACAAAGTGACCCAGCTGCTCCTGCAGCAG
GATAAGGTGCCAGAACCTGCCAGCCTGTCCAGCAATCACAGCCTGACCAGCTGCTTTACCAACCAGGGCTACTTC
TTCTTCCATCTGCCTGACGCTCTCGAGATCGAGGCCTGCCAGGTGTACTTCACCTACGATCCCTACAGCGAGGAA
GATCCCGACGAAGGCGTTGCAGGCGCTCCTACAGGATCTTCTCCACAGCCTCTGCAGCCACTGTCCGGCGAGGAT
GATGCCTACTGCACCTTTCCAAGCCGGGACGACCTGCTGCTGTTTAGCCCTAGTCTGCTCGGCGGACCTTCTCCA
CCATCTACAGCTCCAGGTGGAAGCGGAGCCGGCGAAGAAAGAATGCCTCCTAGCCTGCAAGAGCGGGTGCCCAGA
GATTGGGATCCTCAACCTCTCGGACCTCCTACACCAGGCGTGCCAGACCTGGTGGATTTTCAGCCTCCTCCAGAG
CTGGTGCTGAGAGAAGCTGGCGAAGAAGTGCCAGACGCCGGACCTAGAGAGGGCGTTAGCTTTCCTTGGAGCAGA
CCTCCTGGACAGGGCGAGTTCAGAGCCCTGAATGCTAGACTGCCCCTGAACACCGATGCCTACCTGTCTCTGCAA
GAACTGCAGGGACAAGACCCCACACACCTCGTT (SEQ ID NO: 21)
P2A:
GGCAGCGGCGCCACCAACTTTAGCCTGCTGAAACAAGCCGGCGACGTGGAAGAGAATCCCGGACCT (SEQ ID
NO: 5)
mClover3:
ATGGTGAGCAAGGGCGAGGAGCTGTTCACCGGGGTGGTGCCCATCCTGGTCGAGCTGGACGGCGACGTAAACGGC
CACAAGTTCAGCGTCCGCGGCGAaGGtGAGGGaGATGCCACCAACGGCAAGCTGACCCTGAAGTTCATCTGCACC
ACCGGCAAGCTGCCCGTGCCCTGGCCCACCCTCGTGACCACCTTCGGCTACGGCGTGGCCTGCTTCAGCCGCTAC
CCCGACCACATGAAGCAGCACGACTTCTTCAAGTCCGCCATGCCCGAAGGCTACGTCCAGGAGCGCACCATCTCT
TTCAAGGACGACGGTACCTACAAGACCCGCGCCGAGGTGAAGTTCGAGGGCGACACCCTGGTGAACCGCATCGAG
CTGAAGGGCATCGACTTCAAGGAGGACGGCAACATCCTGGGGCACAAGCTGGAGTACAACTTCAACAGCCACTAC
GTCTATATCACGGCCGACAAGCAGAAGAACTGCATCAAGGCTAACTTCAAGATCCGCCACAACGTTGAGGACGGC
AGCGTGCAGCTCGCCGACCACTACCAGCAGAACACCCCCATCGGCGACGGCCCCGTGCTGCTGCCCGACAACCAC
TACCTGAGCCATCAGTCCAAGCTGAGCAAAGACCCCAACGAGAAGCGCGATCACATGGTCCTGCTGGAGTTCGTG
ACCGCCGCCGGGATTACACATGGCATGGACGAGCTGTACAAGTAA (SEQ ID NO: 6)
Amino acid Sequence:
Leader:
MDWIWRILFLVGAATGAHS (SEQ ID NO: 7)
FLAG:
DYKDDDDK (SEQ ID NO: 8)
cJun:
IARLEEKVKTLKAQNSELASTANMLREQVAQLKQKVMNH (SEQ ID NO: 9)
hIL2Rb:
IPWLGHLLVGLSGAFGFIILVYLLINCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYFFFHLPDALEIEACQVYFTYDPYSEE
DPDEGVAGAPTGSSPQPLQPLSGEDDAYCTFPSRDDLLLFSPSLLGGPSPPSTAPGGSGAGEERMPPSLQERVPR
DWDPQPLGPPTPGVPDLVDFQPPPELVLREAGEEVPDAGPREGVSFPWSRPPGQGEFRALNARLPLNTDAYLSLQ
ELQGQDPTHLV (SEQ ID NO: 22)
P2A:
GSGATNFSLLKQAGDVEENPGP (SEQ ID NO: 11)
mClover3:
MVSKGEELFTGVVPILVELDGDVNGHKFSVRGEGEGDATNGKLTLKFICTTGKLPVPWPTLVTTFGYGVACESRY
PDHMKQHDFFKSAMPEGYVQERTISFKDDGTYKTRAEVKFEGDTLVNRIELKGIDFKEDGNILGHKLEYNENSHY
VYITADKQKNCIKANFKIRHNVEDGSVQLADHYQQNTPIGDGPVLLPDNHYLSHQSKLSKDPNEKRDHMVLLEFV
TAAGITHGMDELYK (SEQ ID NO: 12)
Jun.GHR.MyD88
Nucleotide sequence:
Leader:
ATGGATTGGATCTGGCGGATCCTGTTCCTTGTGGGAGCTGCCACAGGCGCCCACAGC (SEQ ID NO: 1)
FLAG:
GACTACAAGGACGACGACGACAAG (SEQ ID NO: 2)
cJun:
ATCGCCAGGCTGGAGGAGAAGGTGAAGACCCTGAAGGCCCAGAACAGCGAGCTGGCCAGCACCGCCAACATGCTG
AGGGAGCAGGTGGCCCAGCTGAAGCAGAAGGTGATGAACCAC (SEQ ID NO: 3)
Transmembrane domain:
TTCCCCTGGCTGCTGATCATCATCTTCGGCATCTTTGGCCTGACCGTGATGCTGTTCGTGTTCCTGTTCAGC
(SEQ ID NO: 133)
GHR Intracellular domain:
AAGCAGCAGCGGATCAAGATGCTGATCCTGCCTCCTGTGCCTGTGCCTAAGATCAAGGGCATCGACCCCGACCTG
CTGAAAGAGGGCAAGCTGGAAGAAGTGAACACCATCCTGGCCATCCACGACAGCTACAAGCCCGAGTTCCACAGC
GACGATAGCTGGGTCGAGTTCATCGAGCTGGACATCGACGAGCCCGACGAGAAAACCGAGGAAAGCGACACCGAC
AGACTGCTGAGCAGCGACCACGAGAAGTCCCACTCTAACCTGGGCGTGAAGGATGGCGATAGCGGCAGAACAAGC
TGCTGCGAGCCCGATATCCTGGAAACCGACTTCAACGCCAACGACATCCACGAGGGCACCAGCGAAGTTGCCCAG
CCTCAAAGACTGAAGGGCGAAGCCGATCTGCTGTGCCTGGACCAGAAGAACCAGAACAACAGCCCCTACCACGAC
GCCTGTCCTGCTACACAGCAGCCTAGTGTGATCCAGGCCGAGAAGAACAAGCCCCAGCCTCTGCCTACAGAGGGC
GCCGAATCTACACATCAGGCCGCTCACATCCAGCTGAGCAACCCTAGCAGCCTGAGCAACATCGACTTCTACGCC
CAAGTGTCCGACATCACACCAGCCGGATCTGTGGTGCTGTCTCCCGGCCAGAAAAACAAGGCCGGCATGTCCCAG
TGCGACATGCACCCTGAAATGGTGTCCCTGTGCCAAGAGAACTTCCTGATGGACAACGCCTACTTCTGCGAGGCC
GACGCCAAGAAATGCATCCCTGTGGCTCCCCACATCAAGGTGGAAAGCCACATTCAGCCCAGCCTGAATCAAGAG
GATATCTACATCACCACCGAGAGCCTGACCACCGCTGCTGGTAGACCTGGAACAGGCGAGCATGTGCCTGGCTCT
GAAATGCCCGTGCCTGACTACACCAGCATCCACATCGTGCAGAGCCCTCAGGGCCTGATCCTGAATGCTACAGCT
CTGCCCCTGCCAGACAAAGAGTTCCTGTCTAGCTGCGGCTACGTGTCCACCGACCAGCTGAACAAGATCATGCCC
(SEQ ID NO: 43)
MyD88 Intracellular domain:
GCGGCTGGAGGGCCAGGAGCTGGCTCTGCCGCGCCGGTGTCTTCCACCAGTTCACTGCCTTTGGCGGCTCTTAAC
ATGCGGGTCAGGCGAAGATTGAGTTTGTTTCTGAATGTTAGGACTCAAGTAGCTGCCGACTGGACGGCCCTTGCG
GAAGAAATGGACTTCGAGTATCTaGAGATTCGCCAACTTGAAACCCAGGCaGATCCTACAGGCAGACTCCTCGAT
GCGTGGCAGGGTCGGCCTGGAGCATCTGTGGGAAGGTTGCTGGAACTGTTGACTAAACTTGGTCGAGATGACGTA
CTGCTGGAATTGGGTCCAAGCATCGAAGAAGATTGTCAAAAATACATCCTCAAGCAGCAACAAGAGGAAGCAGAG
AAACCATTGCAAGTAGCGGCAGTAGACTCTAGCGTACCGCGCACTGCTGAGCTTGCTGGAATCACGACCCTTGAC
GATCCTCTCGGGCATATGCCAGAGCGGTTCGACGCATTCATATGTTATTGTCCAAGCGATATC (SEQ ID NO:
219)
Amino acid sequence
Leader:
MDWIWRILFLVGAATGAHS (SEQ ID NO: 7)
FLAG :
DYKDDDDK (SEQ ID NO: 8)
cJun:
IARLEEKVKTLKAQNSELASTANMLREQVAQLKQKVMNH (SEQ ID NO: 9)
Transmembrane domain:
FPWLLIIIFGIFGLTVMLEVELES (SEQ ID NO: 52)
GHR Intracellular domain:
KQQRIKMLILPPVPVPKIKGIDPDLLKEGKLEEVNTILAIHDSYKPEFHSDDSWVEFIELDIDEPDEKTEESDTD
RLLSSDHEKSHSNLGVKDGDSGRTSCCEPDILETDENANDIHEGTSEVAQPQRLKGEADLLCLDQKNQNNSPYHD
ACPATQQPSVIQAEKNKPQPLPTEGAESTHQAAHIQLSNPSSLSNIDFYAQVSDITPAGSVVLSPGQKNKAGMSQ
CDMHPEMVSLCQENFLMDNAYFCEADAKKCIPVAPHIKVESHIQPSLNQEDIYITTESLTTAAGRPGTGEHVPGS
EMPVPDYTSIHIVQSPQGLILNATALPLPDKEFLSSCGYVSTDQLNKIMP (SEQ ID NO: 44)
MyD88 Intracellular domain:
AAGGPGAGSAAPVSSTSSLPLAALNMRVRRRLSLFLNVRTQVAADWTALAEEMDFEYLEIRQLETQADPTGRLLD
AWQGRPGASVGRLLELLTKLGRDDVLLELGPSIEEDCQKYILKQQQEEAEKPLQVAAVDSSVPRTAELAGITTLD
DPLGHMPERFDAFICYCPSDI (SEQ ID NO: 220)

Table 3 shows the amino acid sequences of the domains of intracellular region receptor candidates GHR and hEpoR. GHR and hEpoR are non-limiting examples of sources of signaling regions for the intracellular region.

TABLE 3
hEpoR and GHIR regions and constructs

hEpoR constructs
Extracellular domain:
APPPNLPDPKFESKAALLAARGPEELLCFTERLEDLVCFWEEAASAGVGPGNYSFSY
QLEDEPWKLCRLHQAPTARGAVRFWCSLPTADTSSFVPLELRVTAASGAPRYHRVIH
INEVVLLDAPVGLVARLADESGHVVLRWLPPPETPMTSHIRYEVDVSAGNGAGSVQ
RVEILEGRTECVLSNLRGRTRYTFAVRARMAEPSFGGFWSAWSEPVSLLTPSDLDP
(SEQ ID NO: 47)
Transmembrane domain:
LILTLSLILVVILVLLTVLALLS (SEQ ID NO: 48)
Intracellular domain:
HRRALKQKIWPGIPSPESEFEGLFTTHKGNFQLWLYQNDGCLWWSPCTPFTEDPPAS
LEVLSERCWGTMQAVEPGTDDEGPLLEPVGSEHAQDTYLVLDKWLLPRNPPSEDLP
GPGGSVDIVAMDEGSEASSCSSALASKPSPEGASAASFEYTILDPSSQLLRPWTLCPEL
PPTPPHLKYLYLVVSDSGISTDYSSGDSQGAQGGLSDGPYSNPYENSLIPAAEPLPPSY
VACS (SEQ ID NO: 49)
W marks the truncation site for an EpoR intracellular domain mutant with enhanced STAT5
activation (without the Src homology region 2 domain-containing phosphatase-1 (SHP1)
binding site)
Truncated intracellular domain:
HRRALKQKIWPGIPSPESEFEGLFTTHKGNFQLWLYQNDGCLWWSPCTPFTEDPPAS
LEVLSERCWGTMQAVEPGTDDEGPLLEPVGSEHAQDTYLVLDKWLLPRNPPSEDLP
GPGGSVDIVAMDEGSEASSCSSALASKPSPEGASAASFEYTILDPSSQLLRP (SEQ ID
NO: 46)
GHR constructs
Extracellular domain:
FSGSEATAAILSRAPWSLQSVNPGLKTNSSKEPKFTKCRSPERETFSCHWTDEVHHGT
KNLGPIQLFYTRRNTQEWTQEWKECPDYVSAGENSCYFNSSFTSIWIPYCIKLTSNGG
TVDEKCFSVDEIVQPDPPIALNWTLLNVSLTGIHADIQVRWEAPRNADIQKGWMVLE
YELQYKEVNETKWKMMDPILTTSVPVYSLKVDKEYEVRVRSKQRNSGNYGEFSEVL
YVTLPQMSQFTCEEDFY (SEQ ID NO: 51)
Transmembrane domain:
FPWLLIIIFGIFGLTVMLFVFLFS (SEQ ID NO: 52)
Intracellular domain:
KQQRIKMLILPPVPVPKIKGIDPDLLKEGKLEEVNTILAIHDSYKPEFHSDDSWVEFIEL
DIDEPDEKTEESDTDRLLSSDHEKSHSNLGVKDGDSGRTSCCEPDILETDFNANDIHE
GTSEVAQPQRLKGEADLLCLDQKNQNNSPYHDACPATQQPSVIQAEKNKPQPLPTE
GAESTHQAAHIQLSNPSSLSNIDFYAQVSDITPAGSVVLSPGQKNKAGMSQCDMHPE
MVSLCQENFLMDNAYFCEADAKKCIPVAPHIKVESHIQPSLNQEDIYITTESLTTAAG
RPGTGEHVPGSEMPVPDYTSIHIVQSPQGLILNATALPLPDKEFLSSCGYVSTDQLNKI
MP (SEQ ID NO: 44)

### Polynucleotides

In one aspect, the present disclosure provides polynucleotides which may encode any of the chimeric receptors disclosed herein. In some embodiments, the polynucleotide may be a DNA molecule. In some embodiments, the polynucleotide may be an RNA molecule.

In some embodiments, the polynucleotide may comprise a nucleotide sequence that may encode any homodimeric polypeptide disclosed herein.

In some embodiments, the polynucleotide comprises a nucleotide sequence of any of SEQ ID NOs: 111, 113, 115, 117, 119, 121, 123, 125, 147, 149, or 222 (or nucleotides 82-1833 of SEQ ID NO: 222), or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with any of SEQ ID NOs: 111, 113, 115, 117, 119, 121, 123, 125, 147, 149, or 222 (or nucleotides 82-1833 of SEQ ID NO: 222).

In some embodiments, the nucleotide sequence encoding the polypeptide of the chimeric receptor may be operably linked to the one or more additional nucleotide sequences encoding one or more additional polypeptide sequences via a sequence encoding a self-cleaving peptide and/or an internal ribosomal entry site (IRES).

In some embodiments, the self-cleaving peptide may be a 2A peptide. Non-limiting examples of self-cleaving peptide sequences includes Thoseaasigna virus 2A (T2A; AEGRGSLLTCGDVEENPGP (SEQ ID NO: 155), EGRGSLLTCGDVEENPGP (SEQ ID NO: 156), or GSGEGRGSLLTCGDVEENPGP (SEQ ID NO: 157)); the foot and mouth disease virus (FMDV) 2A sequence (F2A; GSGSRVTELLYRMKRAETYCPRPLLAIHPTEARHKQKIVAPVKQLLNFDLLKLAGDV ESNPGP (SEQ ID NO: 158)), Sponge (Amphimedon queenslandica) 2A sequence (LLCFLLLLLSGDVELNPGP (SEQ ID NO: 159); or HHFMFLLLLLAGDIELNPGP (SEQ ID NO: 160)); acorn 2A sequence (Saccoglossus kowalevskii) worm (WFLVLLSFILSGDIEVNPGP (SEQ ID NO: 161)); amphioxus (Branchiostoma floridae) 2A sequence (KNCAMYMLLLSGDVETNPGP (SEQ ID NO: 162); or MVISQLMLKLAGDVEENPGP (SEQ ID NO: 163)); porcine teschovirus-1 2A sequence (P2A; GSGATNFSLLKQAGDVEENPGP (SEQ ID NO: 11)); and equine rhinitis A virus 2A sequence (E2A; GSGQCTNYALLKLAGDVESNPGP (SEQ ID NO: 164)). In some embodiments, the separation sequence is a naturally occurring or synthetic sequence. In certain embodiments, the separation sequence includes the 2A consensus sequence D-X-E-X-NPGP (SEQ ID NO: 165), in which X is any amino acid residue.

In some embodiments, the 2A peptide may be a P2A peptide.

In some embodiments, the P2A peptide comprises the amino acid sequence of SEQ ID NO: 11, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 11. In some embodiments, the nucleotide sequence that encodes the P2A peptide comprises the nucleotide sequence that encodes the amino acid sequence of SEQ ID NO: 11, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 11. In some embodiments, the nucleotide sequence that encodes the P2A peptide comprises the nucleotide sequence set forth in SEQ ID NO: 5, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 5. In some embodiments, the P2A peptide comprises the amino acid sequence set forth in SEQ ID NO: 11. In some embodiments, the nucleotide sequence that encodes P2A peptide comprises the nucleotide sequence set forth in SEQ ID NO: 5.

In some embodiments, the nucleotide sequence(s) may be expressed in an inducible fashion, for example, as may be achieved with an inducible promoter, an inducible expression system, an artificial signaling circuits, and/or drug-induced splicing.

In some embodiments, the nucleotide sequence(s) may be expressed in an inducible fashion, such as that which may be achieved with i) an inducible promoter, for example, but not limited to promotors that may be activated by T cell activation (e.g. NFAT, Nur66, IFNg) or hypoxia; ii) an inducible expression system, for example, but not limited to doxycycline- or tamoxifen-inducible expression system; iii) artificial signaling circuits including, but not limited to, SynNotch, and/or iv) drug-induced splicing. By way of a non-limiting example, drug-induced splicing methods and/or compositions useful in the practice of the present disclosure may be based those described in, for example, Monteys et al., 2021 [44], the contents of which is incorporated herein by reference in its entirety for all purposes.

In some embodiments, the nucleotide sequence(s) may encode a moiety so that the stability of the chimeric receptor may be regulated with a small molecule, including but not limited to, the “SWIFF” technology or an immunomodulatory drug (IMiD)-inducible degron as described, for example, in Juillerat et al., 2019 [17], Carbonneau et al., 2021 [18], and Jan et al., 2021 [19], the contents of each of which is incorporated herein by reference in its entirety for all purposes.

In some embodiments, the nucleotide sequences encoding the polypeptides of the chimeric receptor may be operably linked to at least a regulatory element. The regulatory element may be capable of mediating expression of the polypeptides of the chimeric receptor. Regulatory elements include, but are not limited to, promoters, enhancers, initiation sites, polyadenylation (polyA) tails, IRES elements, response elements, and termination signals. In certain embodiments, the regulatory element regulates chimeric receptor expression. In certain embodiments, the regulatory element increases the expression of the chimeric receptor construct. In certain embodiments, the regulatory element increases the expression of the chimeric receptor construct once the host cell is activated. In certain embodiments, the regulatory element decreases expression of the chimeric receptor construct. In certain embodiments, the regulatory element decreases expression of the chimeric receptor construct once the host cell is activated.

In some embodiments, the promoter is an inducible promoter. Non-limiting examples of an inducible promoter are lac, sp6, T7, and Hsp70- and Hsp90-derived promoters.

In some embodiments, the inducible promoter is a tetracycline (Tc)-inducible promoter.

In some embodiments, the promoter may be a T cell-specific promoter or an NK cell-specific promoter.

### Additional Genes

In addition to the chimeric receptor construct, the polynucleotide may further comprise at least one additional gene that encodes an additional peptide. Examples of additional genes can include a transduced host cell selection marker, an in vivo tracking marker, a cytokine, a suicide gene, or some other functional gene. In certain embodiments, the functional additional gene can induce the expression of another molecule. In certain embodiments, the functional additional gene can increase the safety of the chimeric receptor. For example, the chimeric receptor construct may comprise an additional gene which is truncated CD19 (tCD19). The tCD19 can be used as a tag. Expression of tCD19 may also help determine transduction efficiency. In some embodiments, the functional additional gene may allow for regulation of the stability of the chimeric receptor.

Non-limiting examples of classes of additional genes that can be used to increase the effector function of the modified host cells, include (a) secretable cytokines (e.g., but not limited to, GM-CSF, IL-7, IL-12, IL-15, IL-18), (b) membrane bound cytokines (e.g., but not limited to, IL-15), (c) other chimeric receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T cells (NFATs) (e.g., NFATc1, NFATc2, NFATc3, NFATc4, and NFAT5), (h) antibodies, including fragments thereof and bispecific antibodies (e.g., but not limited to, bispecific T cell engagers (BiTEs)), (i) chimeric antigen receptors (CARs), or (j) safety switches or suicide genes (e.g., CD20, truncated EGFR or HER2, inducible caspase 9 molecules).

In certain embodiments, the chimeric receptor construct may comprise an additional gene that encodes GM-CSF. The expression of exogenous GM-CSF may further enhance the function of the host cells expressing the chimeric receptor of the present disclosure.

In certain embodiments, the chimeric receptor construct may comprise at least one additional gene that encodes, for example, without limitation one or more cellular markers, epitope tags, cytokines, safety switches, dimerization moieties, or degradation moieties.

In some embodiments, the additional gene is an epitope tag. As a non-limiting example, the epitope tag is FLAG. As another non-limiting example, the epitope tag is Myc.

In some embodiments, the additional gene is a cellular marker. As a non-limiting example, the cellular marker is mClover3. As another non-limiting example, the cellular marker is mRuby.

In some embodiments, the additional gene may encode a moiety so that the stability of the chimeric receptor may be regulated with a small molecule, including but not limited to, the “SWIFF” technology or an immunomodulatory drug (IMiD)-inducible degron as described, for example, in Juillerat et al., 2019 [17], Carbonneau et al., 2021 [18], and Jan et al., 2021 [19], the contents of each of which is incorporated herein by reference in its entirety for all purposes.

In certain embodiments, the functional additional gene may be a suicide gene. A suicide gene is a recombinant gene that will cause the host cell that the gene is expressed in to undergo programmed cell death or antibody mediated clearance at a desired time. Suicide genes can function to increase the safety of the chimeric receptor. In another embodiment, the additional gene may be an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105 (11): 4247-4254; US Publ. No. 2011/0286980, each of which are incorporated herein by reference in their entirety for all purposes)).

In certain aspects, chimeric receptors of the present disclosure may be regulated by a safety switch. As used herein, the term “safety switch” refers to any mechanism that is capable of removing or inhibiting the effect of a chimeric receptor from a system (e.g., a culture or a subject). Safety switches can function to increase the safety of the chimeric receptor.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105 (11): 4247-4254; US Publ. No. 2011/0286980, each of which are incorporated herein by reference in their entirety for all purposes).

In some embodiments, the safety switch may be a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the chimeric receptor may be expressed in an inducible fashion, such as that which may be achieved with i) an inducible promoter, for example, but not limited to promotors that may be activated by T cell activation (e.g. NFAT, Nur66, IFNγ) or hypoxia; ii) an inducible expression system, for example, but not limited to doxycycline- or tamoxifen-inducible expression system; iii) artificial signaling circuits including, but not limited to, SynNotch, and/or iv) drug-induced splicing. By way of a non-limiting example, drug-induced splicing methods and/or compositions useful in the practice of the present disclosure may be based those described in, for example, Monteys et al., 2021 [20], the contents of which is incorporated herein by reference in its entirety for all purposes.

In certain embodiments, the chimeric receptor may comprise at least one additional gene (i.e., a second gene). In certain embodiments, the chimeric receptor may comprise one second gene. In other embodiments, the chimeric receptor may comprise two additional genes (i.e., a third gene). In yet another embodiment, the chimeric receptor may comprise three additional genes (i.e., a fourth gene). In certain embodiments, the additional genes may be separated from each other and the chimeric receptor construct. For example, they may be separated by 2A sequences and/or an internal ribosomal entrysites (IRES) as described above. In certain examples, the chimeric receptor may be at any position of the polynucleotide chain.

### Recombinant Vectors

The present disclosure provides recombinant vectors which may comprise a polynucleotide encoding a chimeric receptor. Such recombinant vectors may comprise polynucleotides encoding the proteins disclosed above. In certain embodiments, the polynucleotide may be operatively linked to at least one regulatory element for expression of the chimeric receptor.

In certain embodiments, the vector may be a viral vector. In certain embodiments, the viral vector may be, but is not limited to, a retroviral vector, a lentiviral vector, an adenoviral vector, an adeno-associated virus vector, an alphaviral vector, a herpes virus vector, a baculoviral vector, or a vaccinia virus vector.

In some embodiments, the viral vector may be a retroviral vector.

In some embodiments, the vector may be a non-viral vector. Non-viral vectors suitable for use in this invention include, but are not limited, to minicircle plasmids, transposon systems (e.g., Sleeping Beauty transposon, piggyBac transposon), or single or double stranded DNA molecules that are used as templates for homology directed repair (HDR) based gene editing.

In some embodiments, the vector comprises a nucleotide sequence of any of SEQ ID NOs: 111, 113, 115, 117, 119, 121, 123, 125, 147, 149, or 222, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with any of SEQ ID NOs: 111, 113, 115, 117, 119, 121, 123, 125, 147, 149, or 222.

### Isolated Host Cells

In another aspect, provided herein is an isolated host cell that may comprise any of the various polynucleotides described herein or the recombinant vector described herein.

In a further aspect, provided herein is an isolated host cell that may comprise a chimeric receptor encoded by the polynucleotide described herein.

In certain embodiments, the host cell may be an immune cell. In various embodiments, the host cell is a T cell.

T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+, CD8+T cell, or any other subset of T cells. Other illustrative populations of T cells suitable for use in particular embodiments include naive T cells memory T cells, NKT cells, and iNKT cells.

In some embodiments, the host cell may be a T cell, a natural killer cell (NK) cell, a mesenchymal stem cell (MSC) or a macrophage. In some embodiments, the host cell may be a T cell.

In some embodiments, the T cell may be selected from: an αβ T cell receptor (TCR) T cell, a γδ T cell, a CD8+ T cell, a CD4+ T cell, a cytotoxic T cell, an invariant natural killer T (iNKT) cell, a memory T cell, a memory stem T cell (T_{SCM}), a naïve T cell, an effector T cell, a T-helper cell, or a regulatory T cell (Treg).

In various embodiments, the host cell may be a natural killer (NK) cell. NK cell refers to a differentiated lymphocyte with a CD3-CD16+, CD3-CD56+, CD16+CD56+ and/or CD57+TCR-phenotype.

In some embodiments, the host cell may be an NK cell derived from peripheral, cord blood, IPCs, induced pluripotent stem (iPS) cells (iPSCs), and/or a cell line (e.g., NK-92 cells).

In some embodiments, the host cell may be an immune cell. In some embodiments, the immune cell may be derived from induced pluripotent stem (iPS) cells (iPSCs). Non-limiting examples of iPSCs that may be used in accordance with the disclosure may be any such cells as described in, e.g., Zhu et al., 2019 [21] and Iriguchi et al., 2021 [22] the contents of each of which is incorporated herein by reference in its entirety for all purposes.

In various embodiments, other host immune cells may be selected, for example, but not limited to, macrophages. In various embodiments, the host immune cell may be a dendritic cell, a Langerhans cell, or a B cell. In various embodiments, the host immune cell may be a professional antigen presenting cell (APC). In various embodiments, the host immune cell may be a non-professional antigen presenting cell (APC).

In some embodiments, the host cell may further express one or more antigen-recognition molecules. In some embodiments, the one or more antigen-recognition molecules may be chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

In some embodiments, the host cell may be further genetically modified to enhance its function by a) expressing one or more additional genes (e.g., transcription factors (c-Jun)) or deleting one or more inhibitory genes (e.g., REGNASE-1, DNMT3A) with gene editing technologies (e.g., CRISPR-Cas9 or transcription activator-like effector nucleases (TALENs)). Suitable methods of genetic modification of immune cells to knock out inhibitory genes such as REGNASE-1, and DNMT3A include those described in, e.g., WO2020/219682, WO2020/210365, which are incorporated herein by reference in their entireties.

In various embodiments, the host cell has been activated and/or expanded ex vivo.

In various embodiments, the host cell may be an allogeneic cell. In various embodiments, the host cell may be an autologous cell.

In certain embodiments, the host cell may be isolated form a subject having a cancer. In some embodiments, the host cell may be isolated from a subject having a tumor. In various embodiments, the cancer may be a solid tumor, a brain tumor, or a leukemia. In some embodiments, the tumor may be found within, but not limited to, breast tissue, prostate tissue, bladder tissue, oral and/or dental tissue, head and/or neck tissue, stomach tissue, liver tissue, colorectal tissue, lung tissue, brain tissue, ovary, cervix, esophagus, skin, lymph nodes, and/or bone. In some embodiments, the tumor may be a cancer. In some embodiments, the cancer may be for example, without limitation, osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In certain embodiments, the host cell may be isolated from a subject having a tumor.

In some embodiments, the host cell may be derived from a blood, marrow, tissue, or a tumor sample.

In certain aspects, the present disclosure provides a method of generating an isolated host cell described herein. The method includes genetically modifying the host cell with the polynucleotide described herein or the recombinant vector described herein. In some embodiments, the method may further comprise genetically modifying the host cell to express, one or more antigen-recognition molecules. The one or more antigen-recognition may be selected from chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

In some embodiments, the genetic modifying step may be conducted via viral gene delivery. In some embodiments, the genetic modifying step may be conducted via non-viral gene delivery. In some embodiments, the genetically modifying step may be conducted ex vivo. In some embodiments, the method may further comprise activation and/or expansion of the host cell ex vivo before, after and/or during said genetic modification. In some embodiments, the method may further comprise activation and/or expansion of the host cell ex vivo before, after and/or during genetic modifying step.

In addition to genetically modifying immune cells ex vivo, polynucleotides or recombinant vectors encoding the chimeric receptors described herein could also be prepared ex vivo and directly injected into patients using non-viral or viral delivery approaches (i.e., in vivo gene delivery) (see, e.g., Rurik et al., Science. 2022 Jan. 7; 375 (6576): 91-96, which is incorporated herein by reference in its entirety).

### Isolation/Enrichment

The host cells may be autologous/autogeneic (“self”) or non-autologous (“non-self,” e.g., allogeneic, syngeneic, or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, or HLA-DR, or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, or CD122, or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T cell based host cells can be activated by binding to an agent that activates CD3ζ.

In other embodiments, a CD2-binding agent may be used to provide a primary stimulation signal to the T cells. For example, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above-described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure include, but are not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab) 2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab) 2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD30 agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agent) can be present in a concentration of about 0.1 to about 10 μg/ml. In certain embodiments, the activating agent (e.g., CD3-binding agent) can be present in a concentration of about 0.2 μg/ml to about 9 μg/ml, about 0.3 μg/ml to about 8 μg/ml, about 0.4 μg/ml to about 7 μg/ml, about 0.5 g/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml. In certain embodiments, the CD3-binding agent can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety for all purposes.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK based host cells can be activated by, for example and not limitation, feeder cells (e.g., native K562 cells or K562 cells that are genetically modified to express 4-1BBL and cytokines such as IL-15 or IL-21).

In other embodiments, interferons or macrophage-derived cytokines can be used to activate NK cells. For example, and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

In certain embodiments, the host cells are genetically modified to express a chimeric receptor described above. In certain embodiments, the host cells are further genetically modified to express a CAR, TCR, antibody, or fragment or derivative thereof, disclosed herein. The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the chimeric receptor or other related molecule (e.g., CAR, TCR, antibody, fragment or derivative thereof), the polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue, or organism.

In some embodiments, polynucleotides are transferred to the cell in a non-viral vector. Non-viral vectors suitable for use in this invention include but are not limited to minicircle plasmids, transposon systems (e.g., Sleeping Beauty, piggyBac), or single or double stranded DNA molecules that are used as templates for homology directed repair (HDR) based gene editing.

Nucleic acid vaccines can be used to transfer polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244 (4910): 1342-1344, 1989, both of which are incorporated herein by reference in their entity. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprostatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5 (5): 1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7 (8): 2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87 (9): 3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+ guide RNA) capable of inducing a double strand break in a target DNA sequence is introduced to a host cell, along with a donor polynucleotide encoding a chimeric receptor of the present disclosure and optionally an additional protein (e.g., CAR, TCR, antibody, or fragment or derivative thereof).

### Expansion/Proliferation

After the host cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108 (2): 600-8, Bazdar and Sieg, 2007, Journal of Virology, 2007, 81 (22): 12670-12674, Battalia et al, 2013, Immunology, 139 (1): 109-120). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T cells are CD62L|CD8| specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. Host cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of NK cells can include agents that bind to CD16 or CD56, such as for example αCD16 or αCD56 antibodies. In certain embodiments, the binding agent includes antibodies (see for example Hoshino et al, Blood. 1991 Dec. 15; 78 (12): 3232-40.). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self-discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of an endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule, and/or any other immunogenic molecule in the host cell is disrupted using the CRISPR/Cas technique.

### Pharmaceutical Compositions

In some embodiments, the compositions comprise one or more polypeptides of the chimeric receptor and other related molecules (e.g., CARs, TCRs or antibodies, or fragments or derivatives thereof), polynucleotides, vectors comprising same, and cell compositions, as disclosed herein. Compositions of the present disclosure include, but are not limited to, pharmaceutical compositions.

In some embodiments, the pharmaceutical compositions comprise any of the host cells disclosed herein and a pharmaceutically acceptable carrier and/or excipient.

In one aspect, the present disclosure provides a pharmaceutical composition comprising a polynucleotide or a recombinant vector encoding a chimeric receptor described herein, and a pharmaceutically accepted carrier and/or excipient.

In another aspect, the present disclosure provides pharmaceutical composition comprising the modified host cells comprising a chimeric receptor described herein and a pharmaceutically acceptable carrier and/or excipient.

Examples of pharmaceutical carriers include but are not limited to sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified host cells may be mixed with substances that adhere or penetrate then prior to their administration, e.g., but not limited to, nanoparticles.

### Therapeutic Methods

In one aspect, the present disclosure provides a method of enhancing effector function of an immune cell, which may comprise genetically modifying the cell with the polynucleotide or the recombinant vector encoding a chimeric receptor. In some embodiments, the immune cell may express one or more antigen-recognition molecules selected from: the one or more antigen-recognition molecules are selected from: chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

In some embodiments, the present disclosure provides a method of enhancing effector function an immune cell, wherein the immune cell may express a chimeric antigen receptor (CAR), comprising genetically modifying the cell with the polynucleotide disclosed herein or the recombinant vector disclosed herein.

In some embodiments, the effector function may be one or more of expansion, persistence, and/or cytotoxicity (e.g., anti-tumor activity).

The terms “expand” or “expansion” when used in relation to an immune cell refer to the ability of the immune cell to undergo cellular proliferation (i.e., to increase the number of cells). The terms used herein encompass both in vivo and in vitro immune cell expansion.

The terms “persist” or “persistence” when used in relation to an immune cell refer to the ability of the immune cell (and/or its progenies) to be maintained in a recipient (e.g., a subject) for a period of time. The terms used herein encompass both in vivo and in vitro immune cell persistence.

The term “tumor killing activity” as used herein refers to the ability of an immune cell to inhibit tumor growth and/or to kill the tumor cells (e.g., cancer cells).

In one aspect, the present disclosure provides a method of treating a disease comprising administering to a subject an effective amount of the host cells comprising a chimeric receptor described herein, or the pharmaceutical composition comprising the host cells. In some embodiments, the disease may be cancer such as, but not limited to, a solid tumor. In some embodiments, the disease may be infection. In some embodiments, the disease may be an autoimmune disease.

In one aspect, the present disclosure provides a method for treating a tumor in a subject in need thereof. A therapeutically effective amount of the modified host cells comprising a chimeric receptor described herein or the pharmaceutical composition comprising the host cells is administered to the subject.

The term “tumor” refers to a benign or malignant abnormal growth of tissue. The term “tumor” includes cancer. Examples of tumors include, but are not limited to, soft tissue tumors (e.g., lymphomas), tumors of the blood and blood-forming organs (e.g., leukemias), and solid tumors, which are tumors that grow in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer being treated by methods of the present invention is a HER2 positive cancer. In some embodiments, the HER2 positive cancer is brain, breast, stomach, ovary, uterine serous endometrial carcinoma, colon, bladder, lung, uterine cervix, head and neck, sarcoma, bone tumors, or esophagus cancer.

In some embodiments, the cancer being treated by methods of the present invention is a EphA2 positive cancer. In some embodiments, the EphA2 positive cancer is breast, prostate, urinary bladder, skin, lung, ovary, sarcoma, bone tumors or brain cancer.

In some embodiments, the cancer being treated by methods of the present invention is a B7-H3 positive cancer. In some embodiments, the B7-H3 positive cancer is osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, or melanoma.

In some embodiments, the therapeutic method of the present disclosure may include one or more of the following steps: a) isolating immune cells (e.g., T cells or NK cells) from the subject or donor; b) modifying immune cells (e.g., T cells or NK cells) ex vivo with the polynucleotide or the recombinant vector encoding a chimeric receptor described herein; c) optionally, modifying the immune cells (e.g., T cells or NK cells) ex vivo to express a chimeric antigen receptor (CAR), an antigen specific T cell receptor (TCR) and/or an antibody, or fragment of derivative thereof, and said CAR, TCR or antibody can specifically bind an antigen associated with a disease; d) optionally, expanding and/or activating the modified the immune cells (e.g., T cells or NK cells) before, after, and/or during step b) or c); and e) introducing a therapeutically effective amount of the modified immune cells (e.g., T cells or NK cells) into the subject. In some embodiments, the immune cells express GR-CSF upon activation. In some embodiments, the immune cell is an αβ TCR T cell, a γδ T cell, or an iNKT cell.

In some embodiments, the one or more antigen-recognition may be selected from: chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

In some embodiments, the therapeutic method of the present disclosure may comprise one or more of the following steps: a) isolating T cells or NK cells from the subject or donor; b) modifying said T cells or NK cells ex vivo with a polynucleotide encoding a chimeric receptor described herein or the recombinant vector encoding said polynucleotide; c) optionally, modifying said T cells or NK cells ex vivo to express a chimeric antigen receptor (CAR) that binds an antigen associated with said disease; d) optionally, expanding and/or activating the modified T cells or NK cells before, after and/or during step b) or c); and e) introducing a therapeutically effective amount of the modified T cells or NK cells into the subject.

In some embodiments, the modified host cell is an autologous cell. In some embodiments, the modified host cell is an allogeneic cell. In cases where the host cell is isolated from a donor, the method may further include a method to prevent graft vs host disease (GVHD) and the host cell rejection.

In some embodiments, the modified host cells may also express a CD20 polypeptide as a safety switch. Accordingly, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, compositions comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 107 to 10⁹ cells/kg body weight, or 10⁷ to 10⁸ cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., GM-CSF, IFN or IL-2).

In some embodiments, the method described herein further comprises providing exogenous GM-CSF, in addition to the GM-CSF produced by the immune cells, to enhance the function of immune cells expressing a chimeric receptor of the present disclosure. Exogenous GM-CSF may be provided by, for example and not limitation, i) injection of the FDA-approved GM-CSF drug Sargramostin (Leukine™) or ii) the use of nonviral or viral vectors to express GM-CSF (e.g., FDA-approved GM-CSF expressing oncolytic virus talimogene laherparepvec [TVEC, Imlygic™]). These drugs could be given before, with, or after the administration (e.g., infusion) of the immune cells expressing a chimeric receptor of the present disclosure to patients.

As a non-limiting example, the compositions of the present disclosure can be combined with other therapies that block inflammation (e.g., via blockage of IL-1, IFNα/β, IL-6, TNF, IL-23, etc.).

The methods and compositions of the disclosure can be combined with other immunomodulatory treatments such as, e.g., therapeutic vaccines (including, but not limited to, GVAX, DC-based vaccines, etc.), checkpoint inhibitors (including, but not limited to, agents that block CTLA4, PD1, LAG3, TIM3, etc.) or activators (including, but not limited to, agents that enhance 4-1BB, OX40, etc.). The methods of the invention can be also combined with other treatments that possess the ability to modulate NKT function or stability including, but not limited to, CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CDle). The methods of the invention can also be combined with other treatments such as midostaurin or enasidenib, or a combination thereof.

Therapeutic methods of the disclosure can be combined with additional immunotherapies and therapies. For example, when used for treating tumors, the compositions of the invention can be used in combination with conventional therapies such as, e.g., surgery, radiotherapy, chemotherapy, or combinations thereof, depending on type of the tumor, patient condition, other health issues, and a variety of factors. In certain aspects, other therapeutic agents useful for combination tumor therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The following examples are provided to further describe some of the embodiments disclosed herein. The examples are intended to illustrate, not to limit, the disclosed embodiments.

### Example 1. Jun.MyD88 Homodimers Activate NFκB Signaling and Improve CAR T Cell Antitumor Activity

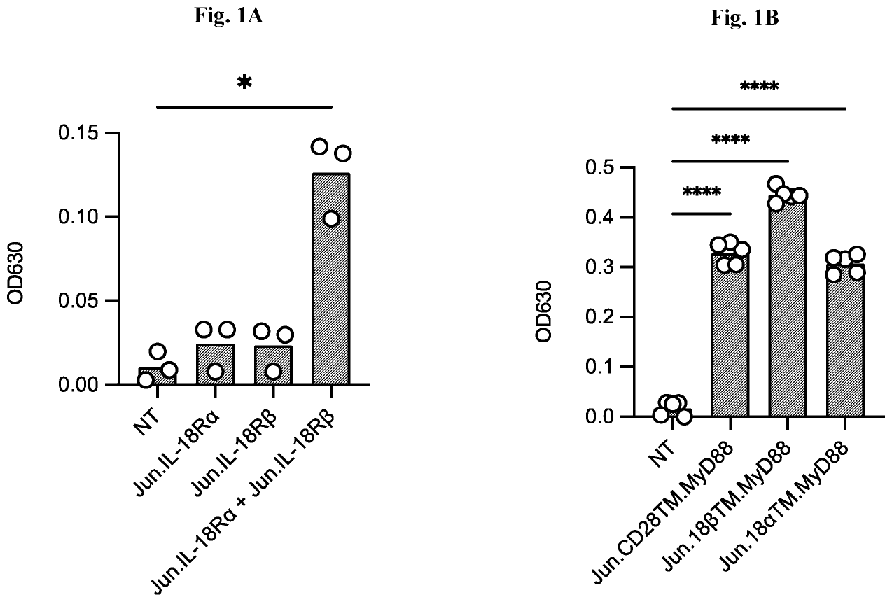

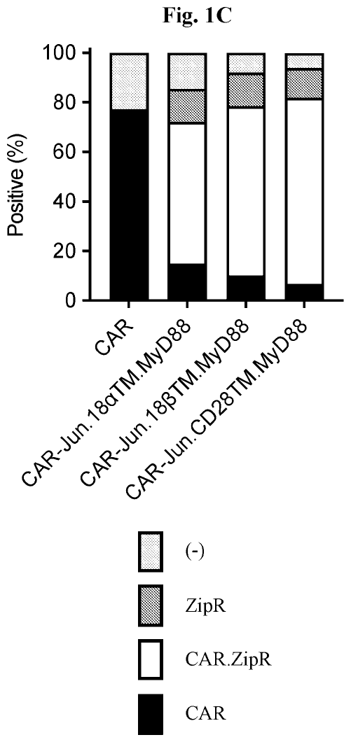

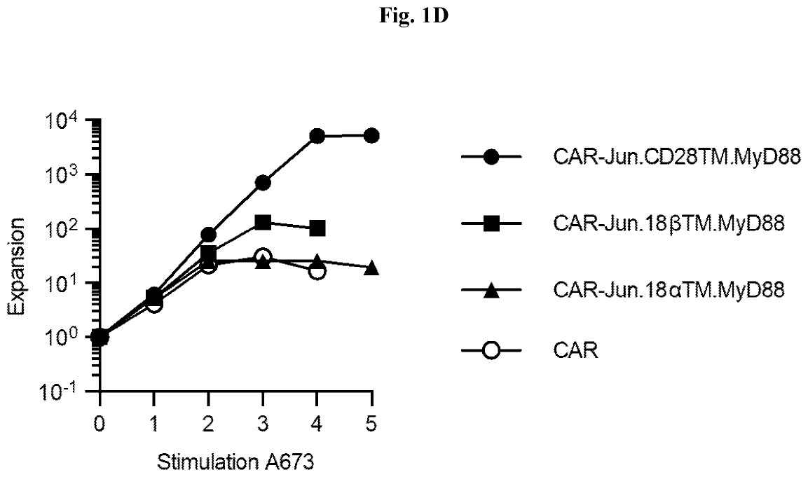

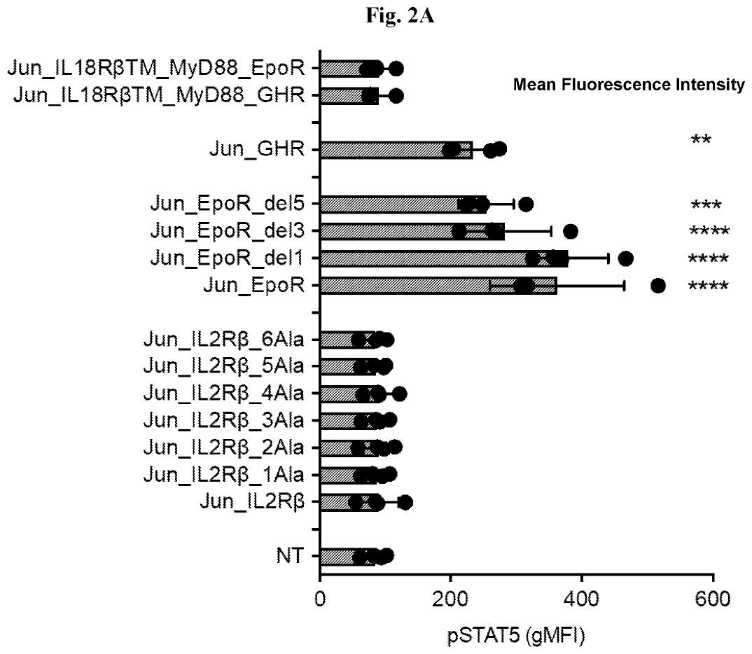

Homodimeric IL-18 ZipRs utilizing either the IL-18Ra chain or IL-18RB chain conjugated to human cJun leucine zipper were generated. These constructs could only activate NFκB signaling in NFκB reporter cells when expressed together, but not as homodimeric molecules (FIG. 1A). IL-18R signaling involves MyD88 aggregation to initiate downstream signal activation, so next human MyD88 was fused to cJun via a CD28 transmembrane (TM) domain (Jun.CD28TM.MyD88), an IL-18Rα chain TM domain (Jun. 18αTM.MyD88), or an IL-18Rβ chain TM domain (Jun. 18βTM.MyD88). All three of these constructs were functional, as judged by NFκB activation in reporter cells (FIG. 1B). When co-expressed with an EphA2-specific CAR in human T cells, all three constructs improved the ability of CAR T cells to repetitively kill A673 tumor cells and expand (FIGS. 1C-1D).

### Example 2. Jun Leucine Zipper-Based Homodimers Activate STAT5 Resulting in Improved T Cell Viability, and are Inhibited by Ruxolitinib

The following constructs were generated and tested:
i) Jun.IL-2Rβ chain constructs with 0 to 6 alanine insertions to interrogate all potential intracellular orientations;
ii) Jun.EpoR constructs with 0, 1, 3, or 5 amino acid TM domain deletions to interrogate receptors with 0, 100, 300, or 500 degrees of rotation;
iii) Jun. GHR; and
iv) Jun. 18B.MyD88 fused to EpoR or GHR (Jun.MyD88.EpoR or Jun.MyD88.GHR).

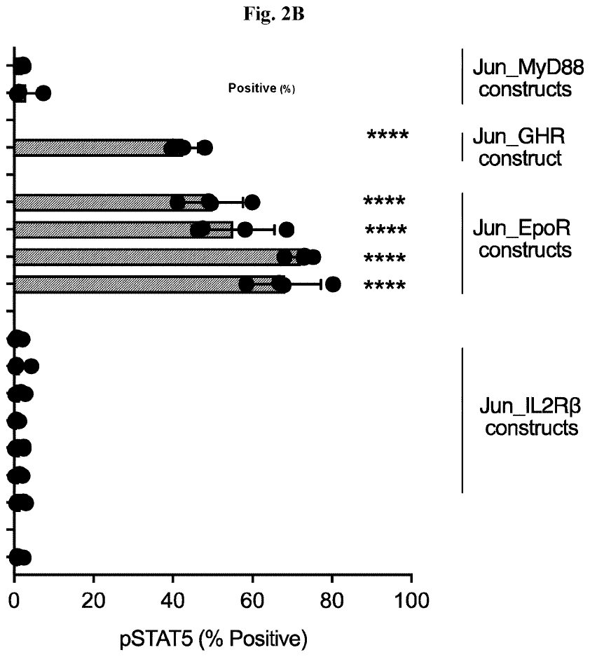

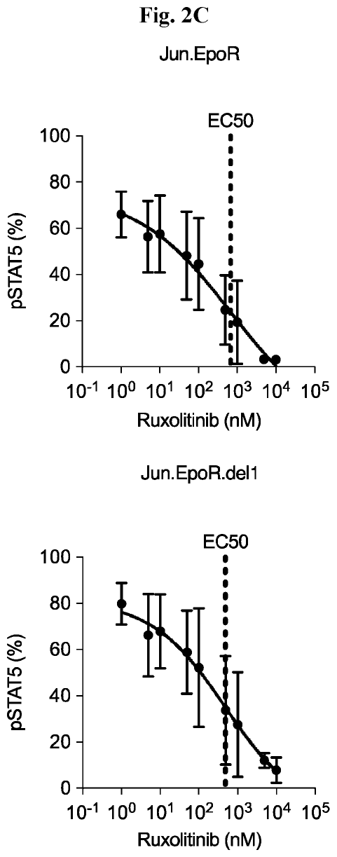

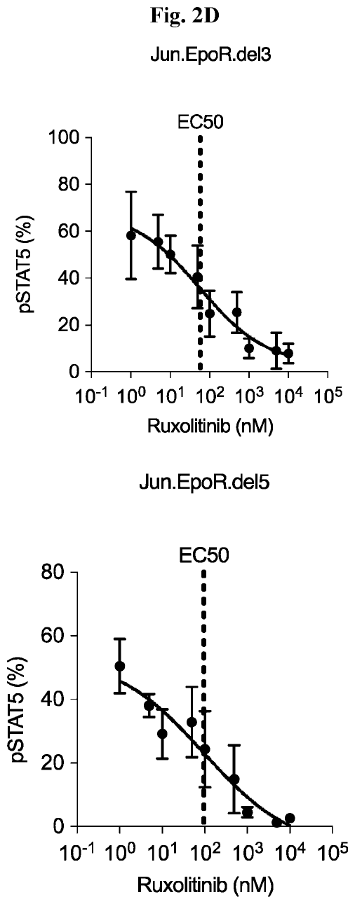

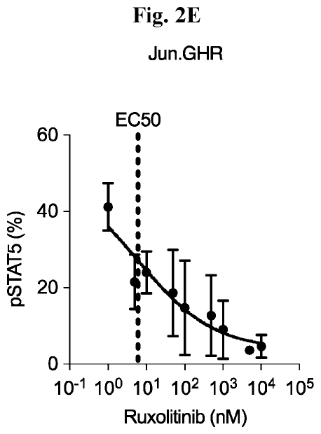

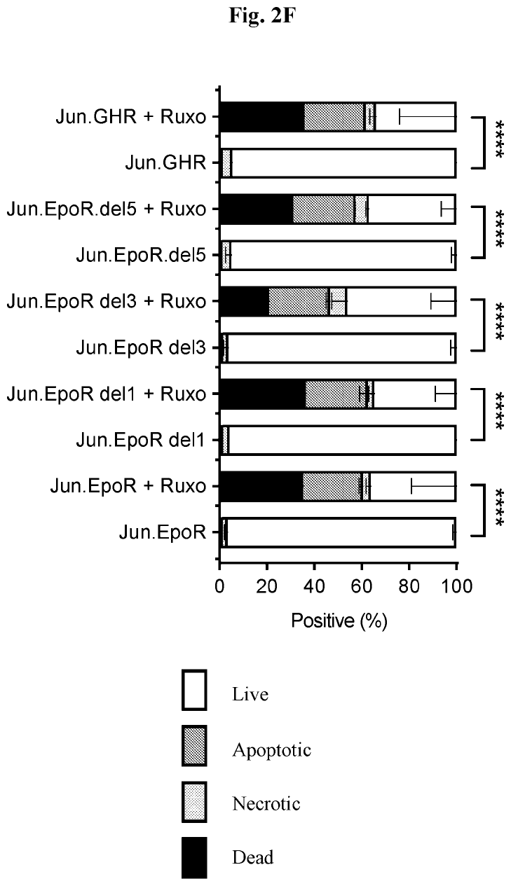

STAT5 signaling pathways improves CAR T cell functionality. When expressed in human T cells, all Jun.EpoR ZipRs and the Jun.GHR ZipR were able to phosphorylate (activate) STAT5 (FIG. 2A). In contrast, the homodimeric Jun.IL-2RB ZipRs and the Jun.MyD88.EpoR or Jun.MyD88.GHR ZipRs did not (FIG. 2B).

As a potential safety switch for the constitutively active homodimeric ZipRs, T cells expressing Jun.EpoR ZipRs or the Jun. GHR ZipRs were treated with increasing concentrations of ruxolitinib, an FDA-approved JAK1/2 inhibitor. All ZipRs were inhibited by ruxolitinib (FIGS. 2C-2E) as judged by their ability to phosphorylate STAT5. The half maximal effective concentration (EC50) of ruxolitinib for ZipRs ranged between 6.3 to 668.3 nM (Table 4), indicating that the signaling strength of each ZipR differ, allowing tailored STAT5 signaling in T cells.

In addition to activation of STAT5, the ability for Jun ZipRs to improve T cell function was determined. Jun.EpoR- or Jun.GHR ZipRs expressing human T cells were cultured without cytokines for 7 days in vitro before quantifying the frequency of live, dead, and dying cells. ZipR signaling allowed cells to survive 7-day cytokine starvation, which was abrogated by concurrent treatment with ruxolitinib (FIG. 2F).

TABLE 4
Ruxolitinib EC50 values

| Construct | EC50 (nM) |
|---|---|
| Jun. EpoR | 668.3 |
| Jun. EpoR. del1 | 483.0 |
| Jun. EpoR. del3 | 57.2 |
| Jun. EpoR. del5 | 94.2 |
| Jun. GHR | 6.3 |

### Example 3. Jun Homodimeric ZipRs do not Alter Human T Cell Immunophenotype

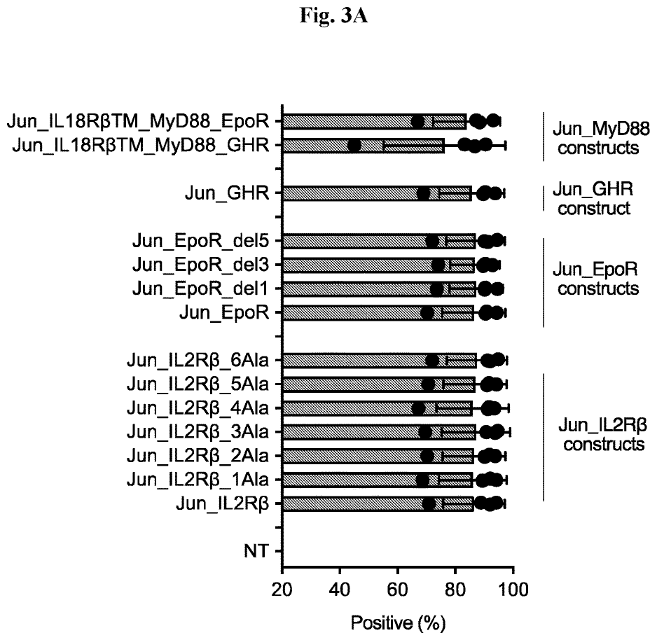

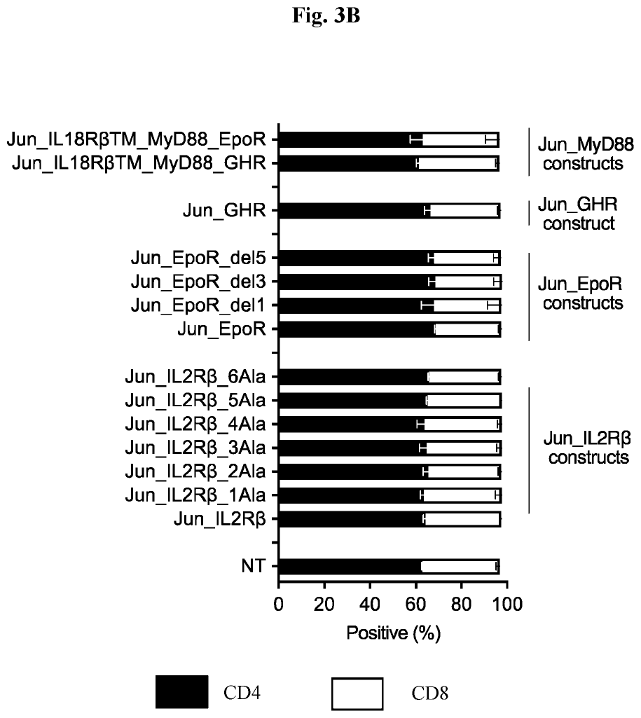

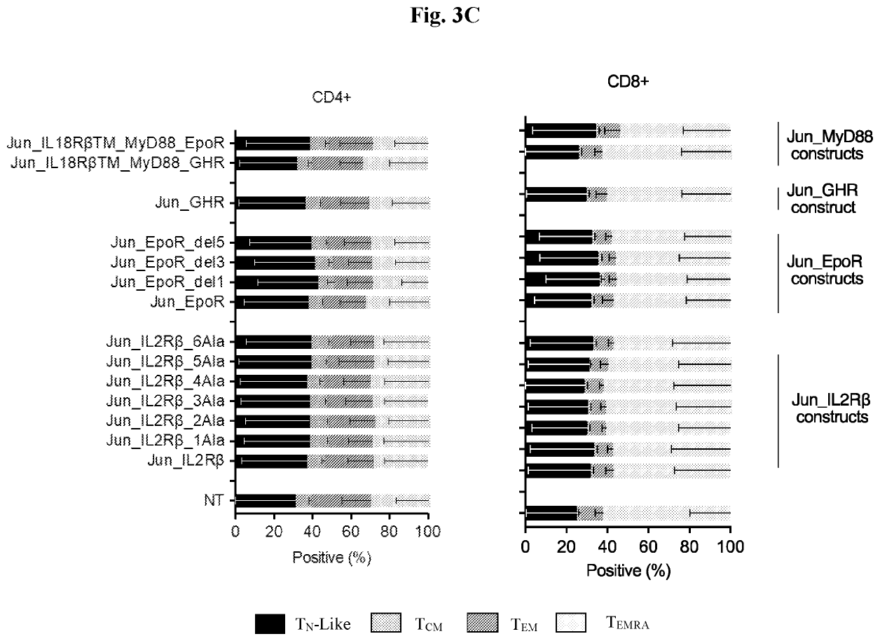

Finally, the effects of ZipR signaling on T cell phenotype and differentiation were evaluated. While ZipRs were expressed in human T cells (FIG. 3A), they did not alter the frequency of CD4+ or CD8+ T cells (FIG. 3B) or their immunophenotype (FIG. 3C).

In summary, the data provided in FIGS. 1-3 demonstrate that generated ZipRs based on EpoR (ZipEpoR) and GHR (ZipGHR) activate STAT5 in a reporter cell line and in primary human T cells.

Below are the methods used in the Examples described above.

Generation of viral vectors. The generation of an RD114-pseudotyped gamma-retroviral vector (pSFG) encoding a 2^{nd} generation EphA2-specific CAR (4H5.CD28z) was previously described. [29,30]. The pSFG vectors encoding Jun. IL2RB, Jun.EpoR, Jun. GHR, Jun.IL18R, and Jun.MyD88 ZipRs were generated by synthesizing gene fragments (Thermo Fisher) by In-Fusion cloning (Takara). They consisted of (i) human cJun (amino acids 277-315) linked to (ii) the intracellular domain of the indicated cytokine receptor chain, (iii) a P2A sequence, and (iv) mClover for detection of transduction. The sequences of the final constructs were verified by sequencing (Hartwell Center, St. Jude Children's Research Hospital). RD114-pseudotyped retroviral particles were generated by transient transfection of 293T cells as previously described⁴. Supernatants were collected after 48 hours, filtered, and snap-frozen.

Generation of EphA2-CAR T cells. Human peripheral blood mononuclear cells (PBMCs) were isolated using Lymphoprep (Abbott Laboratories) from de-identified elutriation chambers of leukapheresis products obtained from St. Jude's donor center. On day 0, PBMCs were stimulated overnight on 24-well non-tissue culture-treated plates that were precoated with CD3 and CD28 antibodies (Miltenyi). On day 1, cytokines IL-7 (10 ng/mL) and IL-15 (5 ng/mL) (PeproTech, Rocky Hill, NJ, USA) were added to the culture. On day 2, T cells were transferred to retronectin-coated (Clontech) plates with retroviral particles for 2 to 4 days. On day 3, T cells were transferred into new 24-well tissue culture-treated plates and subsequently expanded with IL-7 and IL-15. All experiments were performed 7-14 days post-transduction. Biological replicates were performed using PBMCs from different healthy donors.

Generation of ZipR transduced T cells. To transduce T cells with the EphA2-CAR and a ZipR, T cells were transduced as described in the section Generation of EphA2-CAR T Cells, but an admixture of EphA2-CAR and ZipR gamma-retroviral vectors were used. To transduce T cells with only a ZipR, PBMCs were stimulated on plates coated with anti-CD3 and anti-CD28 for 48 hours. Recombinant human IL-7 (10 ng/mL, Peprotech) and IL-15 (5 ng/mL, Peprotech) were added 24 hours after initial stimulation and were maintained in culture until functional studies were performed. Cells were then seeded onto retronectin-coated (Clontech) plates with retroviral particles for 2 to 4 days. T cells were the transferred into new 24-well tissue culture-treated plates and subsequently expanded with IL-7 and IL-15. Nontransduced T cells were prepared similarly, except that no retrovirus was included in the retronectin wells. All experiments were performed 7-14 days post-transduction. Non-transduced (NT) T cells were prepared similarly, except that no retrovirus was included in the retronectin wells. Biological replicates were performed using PBMCs from different healthy donors.

Cell lines. A673 (Ewing sarcoma) was purchased from the American Type Culture Collection (ATCC). Cell lines were authenticated by the ATCC human short-tandem repeat profiling cell authentication service and routinely checked for mycoplasma by the MycoAlert Mycoplasma Detection Kit (Lonza). Once thawed, cell lines were kept in culture for a maximum of 3 months before a new reference vial was thawed. Cell lines were maintained and expanded in Dulbecco's Modified Eagle Medium (GE Healthcare Life Sciences HyClone Laboratories) supplemented with 10% fetal bovine serum (FBS; GE Healthcare Life Sciences HyClone Laboratories) and 2 mmol/L Glutamax (Invitrogen).

Flow Cytometry. A FACSCanto II (BD Biosciences) was used to acquire flow cytometry data, which was analyzed using FlowJo v10 (BD Biosciences). For surface staining, samples were washed with and stained in PBS (Lonza) with 1% FBS (GE Healthcare). For all experiments, matched isotypes or known negatives (e.g., NT T cells) served as gating controls. CAR detection was performed using F(ab′) 2 fragment-specific antibody (polyclonal, Jackson ImmunoResearch, West Grove, PA, USA). T cells were stained with fluorochrome-conjugated antibodies using combinations of the following markers: CD4 (clone SK3, BD Biosciences), CD8 (clone SK1, BD Biosciences), CCR7 (clone 2-L1-A, BD Biosciences), and CD45RO (clone HI100, BD Biosciences).

Intracellular Staining. Intracellular staining to detect pSTAT5 was performed using BD Phosflow Protocol III. Briefly, T cells were unstimulated or stimulated with 5 ng/ml IL-15 for 15 min, then lysed/fixed with 1× Lyse/Fix Buffer (BD Biosciences) for 12 minutes at 37° C. Cells were washed with PBS+1% FBS, then permeabilized with pre-chilled Perm Buffer III (BD Biosciences). Cells were then washed three times before staining with antibodies against pSTAT5 (clone 47, BD Biosciences).

Cytokine starvation. To measure cell survival after 7 days of cytokine withdrawal, 1×10⁶ cells were plated in 1 mL complete RPMI without cytokines in tissue culture treated 48-well plates. After 7 days, cells were collected and stained for flow cytometry. To quantify apoptosis, cells were labeled with annexin V (BD Biosciences) and eBioscience Fixable viability dye (Invitrogen).

Repeat Stimulation Assay. 1×10⁶ T cells were cocultured in complete RPMI with 5×10⁵ tumor cells in a 24-well tissue culture-treated plate. Cells were fed with fresh complete RPMI and split as needed. After 7 days, T cells were harvested, counted, and replated at the same ratio with fresh tumor cells as long as they had killed tumor cells, as determined by microscopic inspection.

### Example 4. Modification of Homochimeric Jun.GHR ZipR for Activation of Additional Signaling Pathways

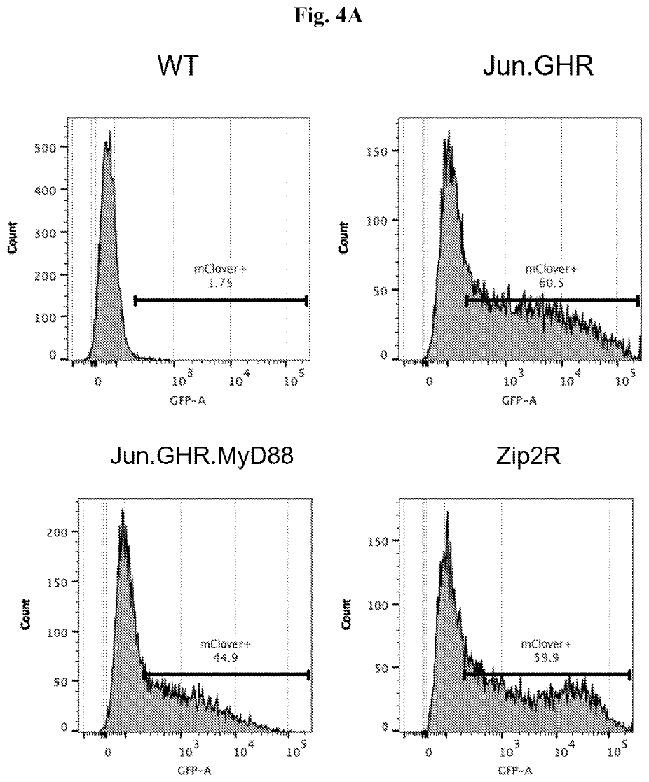

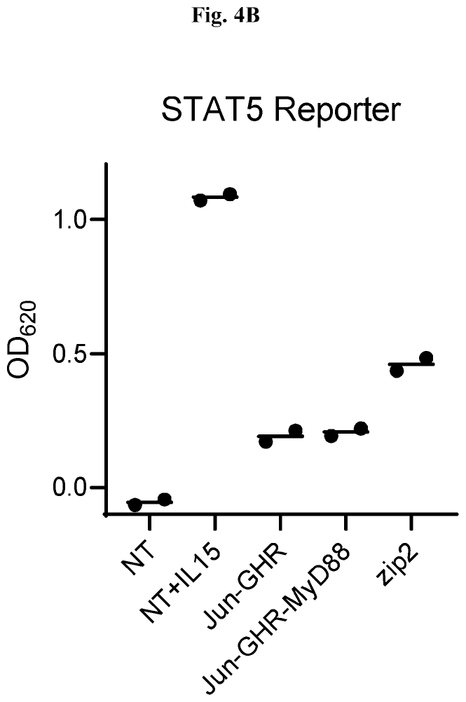

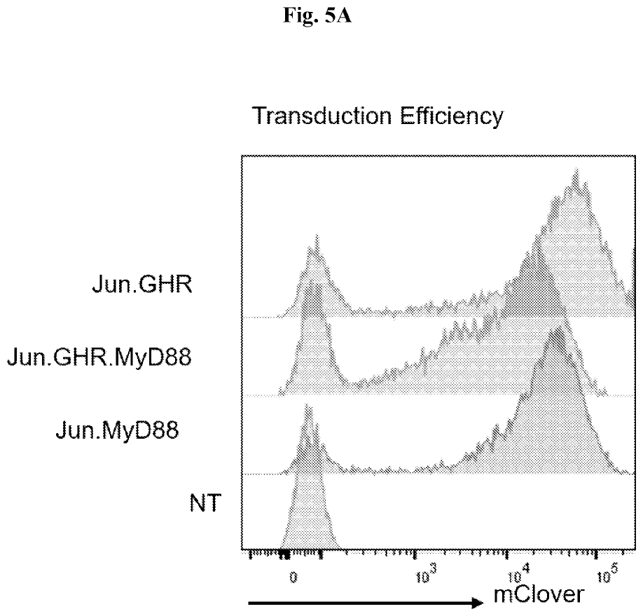

FIGS. 2A-2B of the present disclosure (see also Example 2 above) demonstrate that the homochimeric Jun. GHR ZipR is functional as judged by phosphorylation of STAT5. To demonstrate that this receptor could be further modified to activate additional signaling pathways, a novel molecule in which a MyD88 signaling domain was fused to Jun.GHR ZipR, Jun. GHR.MyD88 ZipR, was generated. The ability of this ZipR (Jun.GHR.MyD88) to activate STAT5 as well as MyD88 signaling pathways was evaluated using reporter cell lines. FIG. 4A demonstrates the STAT5 reporter cell line was successfully transduced with retroviral constructs encoding Jun.GHR and Jun.GHR.MyD88. A retroviral vector encoding an IL-2 receptor-based heterodimeric ZipR (Zip2R; Bell et al, Nat Biomed Eng 2023) was used as a positive control. FIG. 4B demonstrates that Jun.GHR and Jun.GHR.MyD88 phosphorylate STAT5. Thus, addition of the MyD88 domain to Jun. GHR does not interfere with the ability of the GHR domain to phosphorylate STAT5.

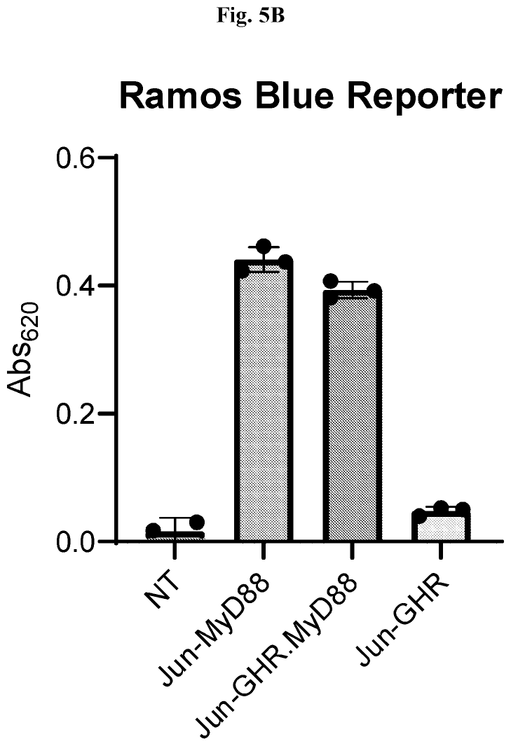

To evaluate the functionality of the MyD88 domain of the Jun. GHR.MyD88 ZipR, the Ramos Blue MyD88 signaling reporter cell line was transduced with Jun.GHR and Jun.GHR.MyD88 (FIG. 5A). Cells transduced with a Jun.MyD88 ZipR served as a positive control. FIG. 5B demonstrates that Jun.GHR.MyD88 activates MyD88 signaling in contrast to Jun.GHR. Thus, the present Example demonstrates for the first time that it is possible to generate a homodimeric ZipR that activates STAT5 and MyD88 signaling pathways. This approach is applicable to EpoR-based homodimeric ZipRs (FIG. 2A) also described herein.

### REFERENCES

1. June C H, Sadelain M. Chimeric Antigen Receptor Therapy. New Engl J Med (2018) 379 (1): 64-73. doi: 10.1056/NEJMra1706169.
2. Srivastava S, Riddell S R. Engineering CAR-T Cells: Design Concepts. Trends Immunol (2015) 36 (8): 494-502. doi: 10.1016/j.it.2015.06.004.
3. Dotti G, Gottschalk S, Savoldo B, Brenner M K. Design and Development of Therapies Using Chimeric Antigen Receptor-Expressing T Cells. Immunol Rev (2014) 257 (1): 107-26. doi: 10.1111/imr. 12131.
4. Juillerat, A, Marechal, A, Filhol, J M et al. Design of chimeric antigen receptors with integrated controllable transient functions. Sci Rep 6, 18950 (2016) doi.org/10.1038/srep18950
5. Leung, W, Gay, J, Martin, U, A. Astrakhan et al., Sensitive, and adaptable pharmacological control of CAR T cells through extracellular receptor dimerization. JCI Insight. 2019; 4 (11): e124430. doi.org/10.1172/jci.insight.124430.
6. Moll J R, Ruvinov S B, Pastan I, Vinson C. Designed heterodimerizing leucine zippers with a ranger of pIs and stabilities up to 10 (-15) M. Protein Sci. 2001 March; 10 (3): 649-55. doi: 10.1110/ps.39401.
7. Reinke A W, Baek J, Ashenberg O, and Keating A E. Networks of bZIP protein-protein interactions diversified over a billion years of evolution. Science. 2013 May 10; 340 (6133): 730-4. doi: 10.1126/science. 1233465.
8. Newman J R S and Keating A E. Comprehensive identification of human bZIP interactions with coiled-coil arrays. Science. June 27; 300 (5628): 2097-101. doi: 2003 10.1126/science. 1084648. Epub 2003 Jun. 12.
9. Mata M, Gerken C, Nguyen P, Krenciute G, Spencer D M, Gottschalk S. Inducible Activation of MyD88 and CD40 in CAR T Cells Results in Controllable and Potent Antitumor Activity in Preclinical Solid Tumor Models. Cancer Discov. 2017 November; 7 (11): 1306-1319. doi: 10.1158/2159-8290.CD-17-0263. Epub 2017 Aug. 11. PMID: 28801306; PMCID: PMC5780189.
10. Katsarou A, Sjöstrand M, Naik J, Mansilla-Soto J, Kefala D, Kladis G, Nianias A, Ruiter R, Poels R, Sarkar I, Patankar Y R, Merino E, Reijmers R M, Frerichs K A, Yuan H, de Bruijn J, Stroopinsky D, Avigan D, van de Donk NWCJ, Zweegman S, Mutis T, Sadelain M, Groen R W J, Themeli M. Combining a CAR and a chimeric costimulatory receptor enhances T cell sensitivity to low antigen density and promotes persistence. Sci Transl Med. 2021 Dec. 8; 13 (623): eabh1962. doi: 10.1126/scitranslmed.abh1962. Epub 2021 Dec. 8. PMID: 34878825; PMCID: PMC9869449.
11. Staerk J, Defour J P, Pecquet C, Leroy E, Antoine-Poirel H, Brett I, Itaya M, Smith S O, Vainchenker W, Constantinescu S N. Orientation-specific signalling by thrombopoietin receptor dimers. EMBO J. 2011 Sep. 2; 30 (21): 4398-413. doi: 10.1038/emboj.2011.315. PMID: 21892137; PMCID: PMC3230373.
12. Kourouniotis G, Wang Y, Pennock S, Chen X, Wang Z. Non-Ligand-Induced Dimerization is Sufficient to Initiate the Signalling and Endocytosis of EGF Receptor. International Journal of Molecular Sciences. 2016; 17 (8): 1200. doi.org/10.3390/ijms17081200.
13. Haifeng Zhang, Puei Nam Tay, Weiping Cao, Wei Li, Jinhua Lu. Integrin-nucleated Toll-like receptor (TLR) dimerization reveals subcellular targeting of TLRs and distinct mechanisms of TLR4 activation and signaling. Febs Letters. 2002, 532 (1-2) 171-176. doi.org/10.1016/S0014-5793 (02) 03669-4.
14. Engelowski, E., Schneider, A., Franke, M. et al. Synthetic cytokine receptors transmit biological signals using artificial ligands. Nat Commun (2018). doi.org/10.1038/s41467-018-04454-8.
15. Mossner, S., Kuchner, M., Modares, N., Knebel, B., Al-Hasani, H., Floss, D., Scheller, J. Synthetic interleukin 22 (IL-22) signaling reveals biological activity of homodimeric IL-10 receptor 2 and functional cross-talk with the IL-6 receptor gp130. Journal of Biological Chemistry, Volume 295, Issue 35, 2020, Pages 12378-12397, ISSN 0021-9258, doi.org/10.1074/jbc.RA120.013927.
16. Stuhlmann-Laeisz, C., Lang, S., Chalaris, A., Krzysztof, P., Enge, S., Eichler, J., Klingmüller, U., Samuel, M., Ernst, M., Rose-John, S., and Jürgen Scheller. Forced Dimerization of gp130 Leads to Constitutive STAT3 Activation, Cytokine-independent Growth, and Blockade of Differentiation of Embryonic Stem Cells. Molecular Biology of the Cell 2006 Vol. 17, No. 7 19 April doi.org/10.1091/mbc.e05-12-1129.
17. Juillerat A, Tkach F, Busser B W, Temburni S, Valton J, Duclert A, Poirot L, Depil S, and Duchateau P. Modulation of chimeric antigen receptor surface expression by a small molecule switch. BMC Biotechnol. 2019; 19:44. Published online 2019 Jul. 3. doi: 10.1186/s12896-019-0537-3.
18. Carbonneau S, Sharma S, Peng L, Rajan V, Hainzl D, Henault M, Yang C, Hale J, Shulok J, Tallarico J, Porter J, Brogdon J L, Dranoff G, Bradner J E, Hild M, and Guimaraes C P. An IMiD-inducible degron provides reversible regulation for chimeric antigen receptor expression and activity. Cell Chemical Biology. Volume 28, Issue 6, 17 Jun. 2021, Pages 802-812.e6
19. Jan M, Scarfò I, Larson R C,Walker A, Schmidts A, Guirguis A A, Gasser J A, Słabicki M, Bouffard A B, Castano A P, Kann M C, Cabral M L, Tepper A, Grinshpun D E, Sperling A S, Kyung T, Sievers Q L, Birnbaum M E, Maus M V, Ebert B L. Reversible O N- and OFF-switch chimeric antigen receptors controlled by lenalidomide. Sci Transl Med. 2021 Jan. 6; 13 (575): eabb6295. doi: 10.1126/scitranslmed.abb6295.
20. Monteys A M, Hundley A A, Ranum P T, Tecedor L, Muehlmatt A, Lim E, Lukashev D, Sivasankaran R, Davidson B L. Regulated control of gene therapies by drug-induced splicing. Nature. 2021 August; 596 (7871): 291-295. doi: 10.1038/s41586-021-03770-2. Epub 2021 Jul. 28.
21. Zhu H and Kaufman D S. An Improved Method to Produce Clinical-Scale Natural Killer Cells from Human Pluripotent Stem Cells. Methods Mol Biol. 2019; 2048:107-119. doi: 10.1007/978-1-4939-9728-2 12.
22. Iriguchi S, Yasui Y, Kawai Y, Arima S, Kunitomo M, Sato T, Ueda T, Minagawa A, Mishima Y, Yanagawa N, Baba Y, Miyake Y, Nakayama K, Takiguchi M, Shinohara T, Nakatsura T, Yasukawa M, Kassai Y, Hayashi A & Kaneko S. A clinically applicable and scalable method to regenerate T-cells from iPSCs for off-the-shelf T-cell immunotherapy. Nature Communications volume 12, Article number: 430 (2021).
23. Musso T, Calosso L, Zucca M, Millesimo M, Ravarino D, Giovarelli M, et al. Human Monocytes Constitutively Express Membrane-Bound, Biologically Active, and Interferon-Gamma-Upregulated Interleukin-15. Blood (1999) 93 (10): 3531-9. doi: 10.1182/blood. V93.10.3531.410k32 3531 3539.
24. Trinchieri G. Interleukin-12 and the Regulation of Innate Resistance and Adaptive Immunity. Nat Rev Immunol (2003) 3 (2): 133-46. doi: 10.1038/nri1001.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A chimeric receptor which is a homodimer comprising two polypeptide monomers, wherein each polypeptide monomer comprises:
i) an extracellular region comprising at least one homodimerizing motif;
ii) a transmembrane region; and
iii) an intracellular region comprising at least one signaling region derived from a cell-surface receptor or a signal transducing adaptor protein, or a portion or variant thereof.

2. The chimeric receptor of claim 1, wherein the extracellular region comprises at least one homodimerizing leucine zipper motif, at least one ligand-dependent homodimerizing motif, or at least one single chain variable fragment, or combinations thereof.

3. The chimeric receptor of claim 1 or 2, wherein the extracellular region comprises at least one homodimerizing leucine zipper motif.

4. The chimeric receptor of claim 3, wherein the homodimerizing leucine zipper motif comprises at least five heptad repeats of amino acids with a leucine at every seventh position.

5. The chimeric receptor of any one of claims 2-4, wherein the homodimerizing leucine zipper motif is derived from transcription factor c-Jun.

6. The chimeric receptor of claim 5, wherein the homodimerizing leucine zipper motif comprises the amino acid sequence of IARLEEKVKTLKAQNSELASTANMLREQVAQLKQKVMNH (SEQ ID NO: 9), or a sequence having at least 80% identity thereto.

7. The chimeric receptor of claim 5, wherein the homodimerizing leucine zipper motif is encoded by the nucleotide sequence of ATCGCCAGGCTGGAGGAGAAGGTGAAGACCCTGAAGGCCCAGAACAGCGAGCTGG CCAGCACCGCCAACATGCTGAGGGAGCAGGTGGCCCAGCTGAAGCAGAAGGTGATG AACCAC (SEQ ID NO: 3), or a sequence having at least 80% identity thereto.

8. The chimeric receptor of claim 2, wherein the ligand-dependent homodimerizing motif undergoes homodimerization in the presence of a chemical inducer.

9. The chimeric receptor of claim 2, wherein the ligand-dependent homodimerizing motif comprises an FKBP12F36V domain.

10. The chimeric receptor of any one of claims 1-9, wherein the signaling region derived from a cell-surface receptor or a signal transducing adaptor protein, or a portion or variant thereof, activates Janus kinase (JAK)-signal transducer and activator of transcription (STAT) pathway, nuclear factor kappa-light-chain-enhancer of activated B cells (NFκB) pathway, and/or additional signaling pathways activated by the MyD88 signaling complex (Myddosome).

11. The chimeric receptor of claim 10, wherein the additional signaling pathways activated by the MyD88 signaling complex (Myddosome) are one or more of interleukin-1 receptor-associated kinase (IRAK), TNF receptor (TNFR)-associated factor (TRAF), TANK-binding kinase (TBK), mitogen-activated protein kinase (MAPK), protein kinase B or AKT (PKB/AKT), and/or phosphatidylinositol-3-kinase (PI3K) pathways.

12. The chimeric receptor of any one of claims 1-11, wherein the intracellular region comprises a first signaling region derived from a cell-surface receptor, or a portion or variant thereof, and a second signaling region derived from a signal transducing adaptor protein, or a portion or variant thereof.

13. The chimeric receptor of any one of claims 1-12, wherein the signaling region is derived from a cytokine receptor, or a portion or variant thereof.

14. The chimeric receptor of claim 13, wherein the cytokine receptor, or a portion or variant thereof, is erythropoietin receptor (EpoR), growth hormone receptor (GHR), prolactin receptor (PRLR), leptin receptor (LEPR), granulocyte colony stimulating factor (G-CSFR), thrombopoietin receptor (TpoR), interleukin-23 receptor (IL-23R), interleukin 10 receptor beta subunit (IL-10R2), IL-6 beta chain (gp130), IL-2RB, IL-18Ra, or IL-18RB.

15. The chimeric receptor of claim 14, wherein the signaling region is derived from EpoR, or a portion or variant thereof.

16. The chimeric receptor of claim 15, wherein the signaling region derived from EpoR does not comprise a Src homology region 2 domain-containing phosphatase-1 (SHP1) binding site.

17. The chimeric receptor of claim 16, wherein the signaling region derived from EpoR comprises the amino acid sequence of HRRALKQKIWPGIPSPESEFEGLFTTHKGNFQLWLYQNDGCLWWSPCTPFTEDPPASLE VLSERCWGTMQAVEPGTDDEGPLLEPVGSEHAQDTYLVLDKWLLPRNPPSEDLPGPGG SVDIVAMDEGSEASSCSSALASKPSPEGASAASFEYTILDPSSQLLRP (SEQ ID NO: 46), or a sequence having at least 80% identity thereto.

18. The chimeric receptor of claim 17, wherein the signaling region derived from EpoR is encoded by the nucleotide sequence of CATAGAAGGGCCCTGAAGCAGAAGATCTGGCCTGGCATCCCATCTCCAGAGAGCGA GTTCGAGGGCCTGTTCACCACACACAAGGGCAACTTCCAGCTGTGGCTGTACCAGA ACGATGGCTGCCTTTGGTGGTCCCCTTGCACACCCTTTACCGAGGATCCACCAGCCA GCCTGGAAGTGCTGAGCGAGAGATGTTGGGGCACAATGCAGGCCGTGGAACCCGGC ACAGATGATGAAGGACCTCTGCTGGAACCTGTGGGCTCTGAACATGCCCAGGACAC CTATCTGGTGCTGGACAAGTGGCTGCTGCCCCGGAATCCTCCATCTGAGGATTTGCC TGGACCTGGCGGCTCCGTGGATATCGTGGCTATGGATGAGGGCAGCGAGGCCAGCT CTTGTTCTTCTGCCCTGGCCAGCAAGCCTTCTCCAGAAGGCGCTTCTGCCGCCAGCTT CGAGTACACCATTCTGGACCCTAGCAGCCAGCTGCTGAGGCCT (SEQ ID NO: 45), or a sequence having at least 80% identity thereto.

19. The chimeric receptor of claim 15, wherein the signaling region derived from EpoR comprises the amino acid sequence of HRRALKQKIWPGIPSPESEFEGLFTTHKGNFQLWLYQNDGCLWWSPCTPFTEDPPASLE VLSERCWGTMQAVEPGTDDEGPLLEPVGSEHAQDTYLVLDKWLLPRNPPSEDLPGPGG SVDIVAMDEGSEASSCSSALASKPSPEGASAASFEYTILDPSSQLLRPWTLCPELPPTPPHL KYLYLVVSDSGISTDYSSGDSQGAQGGLSDGPYSNPYENSLIPAAEPLPPSYVACS (SEQ ID NO: 49), or a sequence having at least 80% identity thereto.

20. The chimeric receptor of claim 19, wherein the signaling region derived from EpoR is encoded by the nucleotide sequence of CATCGCCGCGCGCTGAAACAGAAAATTTGGCCGGGCATTCCGAGCCCGGAAAGCGA ATTTGAAGGCCTGTTTACCACCCATAAAGGCAACTTTCAGCTGTGGCTGTATCAGAA CGATGGCTGCCTGTGGTGGAGCCCGTGCACCCCGTTTACCGAAGATCCGCCGGCGA GCCTGGAAGTGCTGAGCGAACGCTGCTGGGGCACCATGCAGGCGGTGGAACCGGGC ACCGATGATGAAGGCCCGCTGCTGGAACCGGTGGGCAGCGAACATGCGCAGGATAC CTATCTGGTGCTGGATAAATGGCTGCTGCCGCGCAACCCGCCGAGCGAAGATCTGCC GGGCCCGGGCGGCAGCGTGGATATTGTGGCGATGGATGAAGGCAGCGAAGCGAGC AGCTGCAGCAGCGCGCTGGCGAGCAAACCGAGCCCGGAAGGCGCGAGCGCGGCGA GCTTTGAATATACCATTCTGGATCCGAGCAGCCAGCTGCTGCGCCCGTGGACCCTGT GCCCGGAACTGCCGCCGACCCCGCCGCATCTGAAATATCTGTATCTGGTGGTGAGCG ATAGCGGCATTAGCACCGATTATAGCAGCGGCGATAGCCAGGGCGCGCAGGGCGGC CTGAGCGATGGCCCGTATAGCAACCCGTATGAAAACAGCCTGATTCCGGCGGCGGA ACCGCTGCCGCCGAGCTATGTGGCGTGCAGC (SEQ ID NO: 154), or a sequence having at least 80% identity thereto.

21. The chimeric receptor of claim 14, wherein the signaling region is derived from GHR, or a portion or variant thereof.

22. The chimeric receptor of claim 21, wherein the signaling region derived from GHR comprises the amino acid sequence of KQQRIKMLILPPVPVPKIKGIDPDLLKEGKLEEVNTILAIHDSYKPEFHSDDSWVEFIELDI DEPDEKTEESDTDRLLSSDHEKSHSNLGVKDGDSGRTSCCEPDILETDFNANDIHEGTSE VAQPQRLKGEADLLCLDQKNQNNSPYHDACPATQQPSVIQAEKNKPQPLPTEGAESTH QAAHIQLSNPSSLSNIDFYAQVSDITPAGSVVLSPGQKNKAGMSQCDMHPEMVSLCQEN FLMDNAYFCEADAKKCIPVAPHIKVESHIQPSLNQEDIYITTESLTTAAGRPGTGEHVPGS EMPVPDYTSIHIVQSPQGLILNATALPLPDKEFLSSCGYVSTDQLNKIMP (SEQ ID NO: 44), or a sequence having at least 80% identity thereto.

23. The chimeric receptor of claim 22, wherein the signaling region derived from GHR is encoded by the nucleotide sequence of AAGCAGCAGCGGATCAAGATGCTGATCCTGCCTCCTGTGCCTGTGCCTAAGATCAAG GGCATCGACCCCGACCTGCTGAAAGAGGGCAAGCTGGAAGAAGTGAACACCATCCT GGCCATCCACGACAGCTACAAGCCCGAGTTCCACAGCGACGATAGCTGGGTCGAGT TCATCGAGCTGGACATCGACGAGCCCGACGAGAAAACCGAGGAAAGCGACACCGA CAGACTGCTGAGCAGCGACCACGAGAAGTCCCACTCTAACCTGGGCGTGAAGGATG GCGATAGCGGCAGAACAAGCTGCTGCGAGCCCGATATCCTGGAAACCGACTTCAAC GCCAACGACATCCACGAGGGCACCAGCGAAGTTGCCCAGCCTCAAAGACTGAAGGG CGAAGCCGATCTGCTGTGCCTGGACCAGAAGAACCAGAACAACAGCCCCTACCACG ACGCCTGTCCTGCTACACAGCAGCCTAGTGTGATCCAGGCCGAGAAGAACAAGCCC CAGCCTCTGCCTACAGAGGGCGCCGAATCTACACATCAGGCCGCTCACATCCAGCTG AGCAACCCTAGCAGCCTGAGCAACATCGACTTCTACGCCCAAGTGTCCGACATCAC ACCAGCCGGATCTGTGGTGCTGTCTCCCGGCCAGAAAAACAAGGCCGGCATGTCCC AGTGCGACATGCACCCTGAAATGGTGTCCCTGTGCCAAGAGAACTTCCTGATGGACA ACGCCTACTTCTGCGAGGCCGACGCCAAGAAATGCATCCCTGTGGCTCCCCACATCA AGGTGGAAAGCCACATTCAGCCCAGCCTGAATCAAGAGGATATCTACATCACCACC GAGAGCCTGACCACCGCTGCTGGTAGACCTGGAACAGGCGAGCATGTGCCTGGCTC TGAAATGCCCGTGCCTGACTACACCAGCATCCACATCGTGCAGAGCCCTCAGGGCCT GATCCTGAATGCTACAGCTCTGCCCCTGCCAGACAAAGAGTTCCTGTCTAGCTGCGG CTACGTGTCCACCGACCAGCTGAACAAGATCATGCCC (SEQ ID NO: 43), or a sequence having at least 80% identity thereto.

24. The chimeric receptor of any one of claims 1-12, wherein the signaling region is derived from a receptor tyrosine kinase, or a portion or a variant thereof.

25. The chimeric receptor of claim 24, wherein the receptor tyrosine kinase is epidermal growth factor receptor (EGFR).

26. The chimeric receptor of any one of claims 1-12, wherein the signaling region is derived from a Toll-like receptor, or a portion or a variant thereof.

27. The chimeric receptor of claim 26, wherein the Toll-like receptor is TLR1, TLR2, TLR3, TLR4, TLR5, TLR6, TLR7, TLR8, TLR9, or TLR10.

28. The chimeric receptor of any one of claims 1-12, wherein the signaling region is derived from a signal transducing adaptor protein, or a portion or a variant thereof.

29. The chimeric receptor of claim 28, wherein the signal transducing adaptor protein is MyD88.

30. The chimeric receptor of claim 29, wherein the signaling region derived from MyD88 comprises the amino acid sequence of LEAAGGPGAGSAAPVSSTSSLPLAALNMR VRRRLSLFLNVRTQVAADWTALAEEMDFE YLEIRQLETQADPTGRLLDAWQGRPGASVGRLLELLTKLGRDDVLLELGPSIEEDCQKYI LKQQQEEAEKPLQVAAVDSSVPRTAELAGITTLDDPLGHMPERFDAFICYCPSDI (SEQ ID NO: 38) or AAGGPGAGSAAPVSSTSSLPLAALNMRVRRRLSLFLNVRTQVAADWTALAEEMDFEYL EIRQLETQADPTGRLLDAWQGRPGASVGRLLELLTKLGRDDVLLELGPSIEEDCQKYILK QQQEEAEKPLQVAAVDSSVPRTAELAGITTLDDPLGHMPERFDAFICYCPSDI (SEQ ID NO: 220), or a sequence having at least 80% identity thereto.

31. The chimeric receptor of claim 30, wherein the signaling region derived from MyD88 is encoded by the nucleotide sequence of CTCGAGGCGGCTGGAGGGCCAGGAGCTGGCTCTGCCGCGCCGGTGTCTTCCACCAG TTCACTGCCTTTGGCGGCTCTTAACATGCGGGTCAGGCGAAGATTGAGTTTGTTTCTG AATGTTAGGACTCAAGTAGCTGCCGACTGGACGGCCCTTGCGGAAGAAATGGACTT CGAGTATCTAGAGATTCGCCAACTTGAAACCCAGGCAGATCCTACAGGCAGACTCC TCGATGCGTGGCAGGGTCGGCCTGGAGCATCTGTGGGAAGGTTGCTGGAACTGTTG ACTAAACTTGGTCGAGATGACGTACTGCTGGAATTGGGTCCAAGCATCGAAGAAGA TTGTCAAAAATACATCCTCAAGCAGCAACAAGAGGAAGCAGAGAAACCATTGCAAG TAGCGGCAGTAGACTCTAGCGTACCGCGCACTGCTGAGCTTGCTGGAATCACGACCC TTGACGATCCTCTCGGGCATATGCCAGAGCGGTTCGACGCATTCATATGTTATTGTC CAAGCGATATC (SEQ ID NO: 37) or GCGGCTGGAGGGCCAGGAGCTGGCTCTGCCGCGCCGGTGTCTTCCACCAGTTCACTG CCTTTGGCGGCTCTTAACATGCGGGTCAGGCGAAGATTGAGTTTGTTTCTGAATGTT AGGACTCAAGTAGCTGCCGACTGGACGGCCCTTGCGGAAGAAATGGACTTCGAGTA TCTaGAGATTCGCCAACTTGAAACCCAGGCaGATCCTACAGGCAGACTCCTCGATGC GTGGCAGGGTCGGCCTGGAGCATCTGTGGGAAGGTTGCTGGAACTGTTGACTAAAC TTGGTCGAGATGACGTACTGCTGGAATTGGGTCCAAGCATCGAAGAAGATTGTCAA AAATACATCCTCAAGCAGCAACAAGAGGAAGCAGAGAAACCATTGCAAGTAGCGG CAGTAGACTCTAGCGTACCGCGCACTGCTGAGCTTGCTGGAATCACGACCCTTGACG ATCCTCTCGGGCATATGCCAGAGCGGTTCGACGCATTCATATGTTATTGTCCAAGCG ATATC (SEQ ID NO: 219), or a sequence having at least 80% identity thereto.

32. The chimeric receptor of any one of claims 1-31, wherein the transmembrane region is derived from the same cell-surface receptor as the signaling region.

33. The chimeric receptor of claim 32, wherein the transmembrane region is derived from EpoR, or a portion or variant thereof.

34. The chimeric receptor of claim 33, wherein the transmembrane region derived from EpoR comprises the amino acid sequence of LILTLSLILVVILVLLTVLALLS (SEQ ID NO: 48); ILTLSLILVVILVLLTVLALLS (SEQ ID NO: 104), LTLSLILVVILVLLTVLALLS (SEQ ID NO: 128), TLSLILVVILVLLTVLALLS (SEQ ID NO: 106), LSLILVVILVLLTVLALLS (SEQ ID NO: 130), SLILVVILVLLTVLALLS (SEQ ID NO: 108), or LILVVILVLLTVLALLS (SEQ ID NO: 132), or a sequence having at least 80% identity thereto.

35. The chimeric receptor of claim 34, wherein the transmembrane region derived from EpoR is encoded by the nucleotide sequence of CTGATCCTGACACTGAGCCTGATCCTGGTGGTCATCCTGGTGCTGCTGACAGTGCTG GCCCTGCTGTCT (SEQ ID NO: 103), ATCCTGACACTGAGCCTGATCCTGGTGGTCATCCTGGTGCTGCTGACAGTGCTGGCC CTGCTGTCT (SEQ ID NO: 105), CTGACACTGAGCCTGATCCTGGTGGTCATCCTGGTGCTGCTGACAGTGCTGGCCCTG CTGTCT (SEQ ID NO: 127), ACACTGAGCCTGATCCTGGTGGTCATCCTGGTGCTGCTGACAGTGCTGGCCCTGCTG TCT (SEQ ID NO: 107), CTGAGCCTGATCCTGGTGGTCATCCTGGTGCTGCTGACAGTGCTGGCCCTGCTGTCT (SEQ ID NO 129), AGCCTGATCCTGGTGGTCATCCTGGTGCTGCTGACAGTGCTGGCCCTGCTGTCT (SEQ ID NO: 109), or CTGATCCTGGTGGTCATCCTGGTGCTGCTGACAGTGCTGGCCCTGCTGTCT (SEQ ID NO: 131), or a sequence having at least 80% identity thereto.

36. The chimeric receptor of claim 32, wherein the transmembrane region is derived from GHR, or a portion or variant thereof.

37. The chimeric receptor of claim 36, wherein the transmembrane region derived from GHR comprises the amino acid sequence of FPWLLIIIFGIFGLTVMLFVFLFS (SEQ ID NO: 52), PWLLIIIFGIFGLTVMLFVFLFS (SEQ ID NO: 134), WLLIIIFGIFGLTVMLFVFLFS (SEQ ID NO: 136), LLIIIFGIFGLTVMLFVFLFS (SEQ ID NO: 138), LIIIFGIFGLTVMLFVFLFS (SEQ ID NO: 140), IIIFGIFGLTVMLFVFLFS (SEQ ID NO: 142), or IIFGIFGLTVMLFVFLFS (SEQ ID NO: 144), or a sequence having at least 80% identity thereto.

38. The chimeric receptor of claim 37, wherein the transmembrane region derived from GHR is encoded nucleotide by the sequence of TTCCCCTGGCTGCTGATCATCATCTTCGGCATCTTTGGCCTGACCGTGATGCTGTTCG TGTTCCTGTTCAGC (SEQ ID NO: 133), CCCTGGCTGCTGATCATCATCTTCGGCATCTTTGGCCTGACCGTGATGCTGTTCGTGT TCCTGTTCAGC (SEQ ID NO: 135), TGGCTGCTGATCATCATCTTCGGCATCTTTGGCCTGACCGTGATGCTGTTCGTGTTCC TGTTCAGC (SEQ ID NO: 137), CTGCTGATCATCATCTTCGGCATCTTTGGCCTGACCGTGATGCTGTTCGTGTTCCTGT TCAGC (SEQ ID NO: 139), CTGATCATCATCTTCGGCATCTTTGGCCTGACCGTGATGCTGTTCGTGTTCCTGTTCA GC (SEQ ID NO: 141), ATCATCATCTTCGGCATCTTTGGCCTGACCGTGATGCTGTTCGTGTTCCTGTTCAGC (SEQ ID NO: 143), or ATCATCTTCGGCATCTTTGGCCTGACCGTGATGCTGTTCGTGTTCCTGTTCAGC (SEQ ID NO: 145), or a sequence having at least 80% identity thereto.

39. The chimeric receptor of any one of claims 1-31, wherein the transmembrane regions is derived from IL-18Ra, IL-18RB, CD28, IL-2RB, CD8, CD4, CD3ζ, CD40, CD134 (OX-40), CD19, or CD7.

40. The chimeric receptor of claim 39, wherein the transmembrane region is derived from IL-18Ra, or a portion or variant thereof.

41. The chimeric receptor of claim 40, wherein the transmembrane region derived from IL-18Ra comprises the amino acid sequence of MIIAVLILVAVVCLVTVCVI (SEQ ID NO: 40), or a sequence having at least 80% identity thereto.

42. The chimeric receptor of claim 41, wherein the transmembrane region derived from IL-18Ra is encoded by the nucleotide sequence of ATGATCATTGCCGTGCTGATCCTGGTGGCCGTCGTGTGTCTGGTCACCGTGTGCGTG ATC (SEQ ID NO: 39), or a sequence having at least 80% identity thereto.

43. The chimeric receptor of claim 39, wherein the transmembrane region is derived from IL-18RB, or a portion or variant thereof.

44. The chimeric receptor of claim 43, wherein the transmembrane region derived from IL-18RB comprises the amino acid sequence of GVLLYILLGTIGTLVAVLAA (SEQ ID NO: 36), or a sequence having at least 80% identity thereto.

45. The chimeric receptor of claim 44, wherein the transmembrane region derived from IL-18Rβ is encoded by the nucleotide sequence of GGCGTGCTGCTGTACATCCTGCTGGGCACAATCGGAACACTGGTGGCTGTGCTGGCT GCC (SEQ ID NO: 35), or a sequence having at least 80% identity thereto.

46. The chimeric receptor of claim 39, wherein the transmembrane region is derived from CD28, or a portion or variant thereof.

47. The chimeric receptor of claim 46, wherein the transmembrane region derived from CD28 comprises the amino acid sequence of CPSPLFPGPSKPFWVLVVVGGVLACYSLLVTVAFIIFWV (SEQ ID NO: 42), or a sequence having at least 80% identity thereto.

48. The chimeric receptor of claim 47, wherein the transmembrane region derived from CD28 encoded by the nucleotide is sequence of TGCCCAAGTCCTTTGTTTCCGGGTCCTAGCAAGCCGTTCTGGGTCCTCGTTGTCGTAG GAGGAGTGCTTGCGTGCTATAGTCTCCTTGTAACGGTTGCTTTCATTATCTTTTGGGT A (SEQ ID NO: 41), or a sequence having at least 80% identity thereto.

49. The chimeric receptor of any one of claims 1-7, wherein each polypeptide monomer comprises:
i) an extracellular region comprising a homodimerizing leucine zipper motif;
ii) a transmembrane region derived from EpoR, or a portion or variant thereof; and
iii) an intracellular region comprising a signaling region derived from EpoR, or a portion or variant thereof.

50. The chimeric receptor of claim 49, wherein the signaling region derived from EpoR does not comprise the Src homology region 2 domain-containing phosphatase-1 (SHP1) binding site.

51. The chimeric receptor of claim 50, wherein the signaling region derived from EpoR comprises the amino acid sequence of HRRALKQKIWPGIPSPESEFEGLFTTHKGNFQLWLYQNDGCLWWSPCTPFTEDPPASLE VLSERCWGTMQAVEPGTDDEGPLLEPVGSEHAQDTYLVLDKWLLPRNPPSEDLPGPGG SVDIVAMDEGSEASSCSSALASKPSPEGASAASFEYTILDPSSQLLRP (SEQ ID NO: 46), or a sequence having at least 80% identity thereto.

52. The chimeric receptor of claim 51, wherein the signaling region derived from EpoR is encoded nucleotide by the sequence of CATAGAAGGGCCCTGAAGCAGAAGATCTGGCCTGGCATCCCATCTCCAGAGAGCGA GTTCGAGGGCCTGTTCACCACACACAAGGGCAACTTCCAGCTGTGGCTGTACCAGA ACGATGGCTGCCTTTGGTGGTCCCCTTGCACACCCTTTACCGAGGATCCACCAGCCA GCCTGGAAGTGCTGAGCGAGAGATGTTGGGGCACAATGCAGGCCGTGGAACCCGGC ACAGATGATGAAGGACCTCTGCTGGAACCTGTGGGCTCTGAACATGCCCAGGACAC CTATCTGGTGCTGGACAAGTGGCTGCTGCCCCGGAATCCTCCATCTGAGGATTTGCC TGGACCTGGCGGCTCCGTGGATATCGTGGCTATGGATGAGGGCAGCGAGGCCAGCT CTTGTTCTTCTGCCCTGGCCAGCAAGCCTTCTCCAGAAGGCGCTTCTGCCGCCAGCTT CGAGTACACCATTCTGGACCCTAGCAGCCAGCTGCTGAGGCCT (SEQ ID NO: 45), or a sequence having at least 80% identity thereto.

53. The chimeric receptor of any one of claims 49-52, wherein the transmembrane region derived from EpoR comprises the amino acid sequence of LILTLSLILVVILVLLTVLALLS (SEQ ID NO: 48), ILTLSLILVVILVLLTVLALLS (SEQ ID NO: 104), LTLSLILVVILVLLTVLALLS (SEQ ID NO: 128), TLSLILVVILVLLTVLALLS (SEQ ID NO: 106), LSLILVVILVLLTVLALLS (SEQ ID NO: 130), SLILVVILVLLTVLALLS (SEQ ID NO: 108), or LILVVILVLLTVLALLS (SEQ ID NO: 132), or a sequence having at least 80% identity thereto.

54. The chimeric receptor of claim 53, wherein the transmembrane region derived from EpoR is encoded the by nucleotide sequence of CTGATCCTGACACTGAGCCTGATCCTGGTGGTCATCCTGGTGCTGCTGACAGTGCTG GCCCTGCTGTCT (SEQ ID NO: 103), ATCCTGACACTGAGCCTGATCCTGGTGGTCATCCTGGTGCTGCTGACAGTGCTGGCC CTGCTGTCT (SEQ ID NO: 105), CTGACACTGAGCCTGATCCTGGTGGTCATCCTGGTGCTGCTGACAGTGCTGGCCCTG CTGTCT (SEQ ID NO: 127), ACACTGAGCCTGATCCTGGTGGTCATCCTGGTGCTGCTGACAGTGCTGGCCCTGCTG TCT (SEQ ID NO: 107), CTGAGCCTGATCCTGGTGGTCATCCTGGTGCTGCTGACAGTGCTGGCCCTGCTGTCT (SEQ ID NO: 129), AGCCTGATCCTGGTGGTCATCCTGGTGCTGCTGACAGTGCTGGCCCTGCTGTCT (SEQ ID NO: 109), or CTGATCCTGGTGGTCATCCTGGTGCTGCTGACAGTGCTGGCCCTGCTGTCT (SEQ ID NO: 131), or a sequence having at least 80% identity thereto.

55. The chimeric receptor of any one of claims 49-54, wherein each polypeptide monomer comprises the amino acid sequence of SEQ ID NO: 112, 114, 116, or 118, or a sequence having at least 80% identity thereto.

56. The chimeric receptor of claim 55, wherein each polypeptide monomer is encoded by the nucleotide sequence of SEQ ID NO: 111, 113, 115, or 117, or a sequence having at least 80% identity thereto.

57. The chimeric receptor of any one of claims 1-7, wherein each polypeptide monomer comprises:
i) an extracellular region comprising a homodimerizing leucine zipper motif;
ii) a transmembrane region derived from GHR, or a portion or variant thereof; and
iii) an intracellular region comprising a signaling region derived from GHR, or a portion or variant thereof.

58. The chimeric receptor of claim 57, wherein the signaling region derived from GHR comprises the amino acid sequence of KQQRIKMLILPPVPVPKIKGIDPDLLKEGKLEEVNTILAIHDSYKPEFHSDDSWVEFIELDI DEPDEKTEESDTDRLLSSDHEKSHSNLGVKDGDSGRTSCCEPDILETDFNANDIHEGTSE VAQPQRLKGEADLLCLDQKNQNNSPYHDACPATQQPSVIQAEKNKPQPLPTEGAESTH QAAHIQLSNPSSLSNIDFYAQVSDITPAGSVVLSPGQKNKAGMSQCDMHPEMVSLCQEN FLMDNAYFCEADAKKCIPVAPHIKVESHIQPSLNQEDIYITTESLTTAAGRPGTGEHVPGS EMPVPDYTSIHIVQSPQGLILNATALPLPDKEFLSSCGYVSTDQLNKIMP (SEQ ID NO: 44), or a sequence having at least 80% identity thereto.

59. The chimeric receptor of claim 58, wherein the signaling region derived from GHR is encoded by the nucleotide sequence of AAGCAGCAGCGGATCAAGATGCTGATCCTGCCTCCTGTGCCTGTGCCTAAGATCAAG GGCATCGACCCCGACCTGCTGAAAGAGGGCAAGCTGGAAGAAGTGAACACCATCCT GGCCATCCACGACAGCTACAAGCCCGAGTTCCACAGCGACGATAGCTGGGTCGAGT TCATCGAGCTGGACATCGACGAGCCCGACGAGAAAACCGAGGAAAGCGACACCGA CAGACTGCTGAGCAGCGACCACGAGAAGTCCCACTCTAACCTGGGCGTGAAGGATG GCGATAGCGGCAGAACAAGCTGCTGCGAGCCCGATATCCTGGAAACCGACTTCAAC GCCAACGACATCCACGAGGGCACCAGCGAAGTTGCCCAGCCTCAAAGACTGAAGGG CGAAGCCGATCTGCTGTGCCTGGACCAGAAGAACCAGAACAACAGCCCCTACCACG ACGCCTGTCCTGCTACACAGCAGCCTAGTGTGATCCAGGCCGAGAAGAACAAGCCC CAGCCTCTGCCTACAGAGGGCGCCGAATCTACACATCAGGCCGCTCACATCCAGCTG AGCAACCCTAGCAGCCTGAGCAACATCGACTTCTACGCCCAAGTGTCCGACATCAC ACCAGCCGGATCTGTGGTGCTGTCTCCCGGCCAGAAAAACAAGGCCGGCATGTCCC AGTGCGACATGCACCCTGAAATGGTGTCCCTGTGCCAAGAGAACTTCCTGATGGACA ACGCCTACTTCTGCGAGGCCGACGCCAAGAAATGCATCCCTGTGGCTCCCCACATCA AGGTGGAAAGCCACATTCAGCCCAGCCTGAATCAAGAGGATATCTACATCACCACC GAGAGCCTGACCACCGCTGCTGGTAGACCTGGAACAGGCGAGCATGTGCCTGGCTC TGAAATGCCCGTGCCTGACTACACCAGCATCCACATCGTGCAGAGCCCTCAGGGCCT GATCCTGAATGCTACAGCTCTGCCCCTGCCAGACAAAGAGTTCCTGTCTAGCTGCGG CTACGTGTCCACCGACCAGCTGAACAAGATCATGCCC (SEQ ID NO: 43), or a sequence having at least 80% identity thereto.

60. The chimeric receptor of any one of claims 57-59, wherein the transmembrane region derived from GHR comprises the amino acid sequence of FPWLLIIIFGIFGLTVMLFVFLFS (SEQ ID NO: 52), PWLLIIIFGIFGLTVMLFVFLFS (SEQ ID NO: 134), WLLIIIFGIFGLTVMLFVFLFS (SEQ ID NO: 136), LLIIIFGIFGLTVMLFVFLFS (SEQ ID NO: 138), LIIIFGIFGLTVMLFVFLFS (SEQ ID NO: 140), IIIFGIFGLTVMLFVFLFS (SEQ ID NO: 142), or IIFGIFGLTVMLFVFLFS (SEQ ID NO: 144), or a sequence having at least 80% identity thereto.

61. The chimeric receptor of claim 60, wherein the transmembrane region derived from GHR is encoded the by nucleotide sequence of
(SEQ ID NO: 133)
TTCCCCTGGCTGCTGATCATCATCTTCGGCATCTTTGGCCTGACCGTGA
TGCTGTTCGTGTTCCTGTTCAGC,
(SEQ ID NO: 135)
CCCTGGCTGCTGATCATCATCTTCGGCATCTTTGGCCTGACCGTGATGC
TGTTCGTGTTCCTGTTCAGC,
(SEQ ID NO: 137)
TGGCTGCTGATCATCATCTTCGGCATCTTTGGCCTGACCGTGATGCTGT
TCGTGTTCCTGTTCAGC,
(SEQ ID NO: 139)
CTGCTGATCATCATCTTCGGCATCTTTGGCCTGACCGTGATGCTGTTCG
TGTTCCTGTTCAGC,
(SEQ ID NO: 141)
CTGATCATCATCTTCGGCATCTTTGGCCTGACCGTGATGCTGTTCGTGT
TCCTGTTCAGC,
(SEQ ID NO: 143)
ATCATCATCTTCGGCATCTTTGGCCTGACCGTGATGCTGTTCGTGTTCC
TGTTCAGC,
or
(SEQ ID NO: 145)
ATCATCTTCGGCATCTTTGGCCTGACCGTGATGCTGTTCGTGTTCCTGT
TCAGC.

62. The chimeric receptor of any one of claims 57-61, wherein each polypeptide monomer comprises the amino acid sequence of SEQ ID NO: 120, or a sequence having at least 80% identity thereto.

63. The chimeric receptor of claim 62, wherein each polypeptide monomer is encoded by the nucleotide sequence of SEQ ID NO: 119, or a sequence having at least 80% identity thereto.

64. The chimeric receptor of any one of claims 1-7, wherein each polypeptide monomer comprises:
i) an extracellular region comprising a homodimerizing leucine zipper motif;
ii) a transmembrane region derived from IL-18Ra, IL-18Rß or CD28, or a portion or variant thereof; and
iii) an intracellular region comprising a signaling region derived from MyD88, or a portion or variant thereof.

65. The chimeric receptor of claim 64, wherein the signaling region derived from MyD88 comprises the amino acid sequence of LEAAGGPGAGSAAPVSSTSSLPLAALNMRVRRRLSLFLNVRTQVAADWTALAEEMDFE YLEIRQLETQADPTGRLLDAWQGRPGASVGRLLELLTKLGRDDVLLELGPSIEEDCQKYI LKQQQEEAEKPLQVAAVDSSVPRTAELAGITTLDDPLGHMPERFDAFICYCPSDI (SEQ ID NO: 38), or AAGGPGAGSAAPVSSTSSLPLAALNMR VRRRLSLFLNVRTQVAADWTALAEEMDFEYL EIRQLETQADPTGRLLDAWQGRPGASVGRLLELLTKLGRDDVLLELGPSIEEDCQKYILK QQQEEAEKPLQVAAVDSSVPRTAELAGITTLDDPLGHMPERFDAFICYCPSDI (SEQ ID NO: 220), or a sequence having at least 80% identity thereto.

66. The chimeric receptor of claim 65, wherein the signaling region derived from MyD88 is encoded by the nucleotide sequence of CTCGAGGCGGCTGGAGGGCCAGGAGCTGGCTCTGCCGCGCCGGTGTCTTCCACCAG TTCACTGCCTTTGGCGGCTCTTAACATGCGGGTCAGGCGAAGATTGAGTTTGTTTCTG AATGTTAGGACTCAAGTAGCTGCCGACTGGACGGCCCTTGCGGAAGAAATGGACTT CGAGTATCTAGAGATTCGCCAACTTGAAACCCAGGCAGATCCTACAGGCAGACTCC TCGATGCGTGGCAGGGTCGGCCTGGAGCATCTGTGGGAAGGTTGCTGGAACTGTTG ACTAAACTTGGTCGAGATGACGTACTGCTGGAATTGGGTCCAAGCATCGAAGAAGA TTGTCAAAAATACATCCTCAAGCAGCAACAAGAGGAAGCAGAGAAACCATTGCAAG TAGCGGCAGTAGACTCTAGCGTACCGCGCACTGCTGAGCTTGCTGGAATCACGACCC TTGACGATCCTCTCGGGCATATGCCAGAGCGGTTCGACGCATTCATATGTTATTGTC CAAGCGATATC (SEQ ID NO: 37) or GCGGCTGGAGGGCCAGGAGCTGGCTCTGCCGCGCCGGTGTCTTCCACCAGTTCACTG CCTTTGGCGGCTCTTAACATGCGGGTCAGGCGAAGATTGAGTTTGTTTCTGAATGTT AGGACTCAAGTAGCTGCCGACTGGACGGCCCTTGCGGAAGAAATGGACTTCGAGTA TCTaGAGATTCGCCAACTTGAAACCCAGGCaGATCCTACAGGCAGACTCCTCGATGC GTGGCAGGGTCGGCCTGGAGCATCTGTGGGAAGGTTGCTGGAACTGTTGACTAAAC TTGGTCGAGATGACGTACTGCTGGAATTGGGTCCAAGCATCGAAGAAGATTGTCAA AAATACATCCTCAAGCAGCAACAAGAGGAAGCAGAGAAACCATTGCAAGTAGCGG CAGTAGACTCTAGCGTACCGCGCACTGCTGAGCTTGCTGGAATCACGACCCTTGACG ATCCTCTCGGGCATATGCCAGAGCGGTTCGACGCATTCATATGTTATTGTCCAAGCG ATATC (SEQ ID NO: 219), or a sequence having at least 80% identity thereto.

67. The chimeric receptor of any one of claims 64-66, wherein the transmembrane region comprises the amino acid sequence of MIIAVLILVAVVCLVTVCVI (SEQ ID NO: 40), GVLLYILLGTIGTLVAVLAA (SEQ ID NO: 36), or CPSPLFPGPSKPFWVLVVVGGVLACYSLLVTVAFIIFWV (SEQ ID NO: 42), or a sequence having at least 80% identity thereto.

68. The chimeric receptor of claim 67, wherein the transmembrane region is encoded by the nucleotide sequence of ATGATCATTGCCGTGCTGATCCTGGTGGCCGTCGTGTGTCTGGTCACCGTGTGCGTG ATC (SEQ ID NO: 39), GGCGTGCTGCTGTACATCCTGCTGGGCACAATCGGAACACTGGTGGCTGTGCTGGCT GCC (SEQ ID NO: 35), or TGCCCAAGTCCTTTGTTTCCGGGTCCTAGCAAGCCGTTCTGGGTCCTCGTTGTCGTAG GAGGAGTGCTTGCGTGCTATAGTCTCCTTGTAACGGTTGCTTTCATTATCTTTTGGGT A (SEQ ID NO: 41), or a sequence having at least 80% identity thereto.

69. The chimeric receptor of any one of claims 64-68, wherein each polypeptide monomer comprises the amino acid sequence of SEQ ID NO: 122, 124, or 126, or a sequence having at least 80% identity thereto.

70. The chimeric receptor of claim 69, wherein each polypeptide monomer is encoded by the nucleotide sequence of SEQ ID NO: 121, 123, or 125, or a sequence having at least 80% identity thereto.

71. The chimeric receptor of any one of claims 1-7, wherein each polypeptide monomer comprises:
i) an extracellular region comprising a homodimerizing leucine zipper motif;
ii) a transmembrane region derived from GHR, or a portion or variant thereof, and
iii) an intracellular region comprising a signaling region derived from GHR, or a portion or variant thereof, and a signaling region derived from MyD88, or a portion or variant thereof.

72. The chimeric receptor of claim 71, wherein the signaling region derived from GHR comprises the amino acid sequence of KQQRIKMLILPPVPVPKIKGIDPDLLKEGKLEEVNTILAIHDSYKPEFHSDDSWVEFIELDI DEPDEKTEESDTDRLLSSDHEKSHSNLGVKDGDSGRTSCCEPDILETDFNANDIHEGTSE VAQPQRLKGEADLLCLDQKNQNNSPYHDACPATQQPSVIQAEKNKPQPLPTEGAESTH QAAHIQLSNPSSLSNIDFYAQVSDITPAGSVVLSPGQKNKAGMSQCDMHPEMVSLCQEN FLMDNAYFCEADAKKCIPVAPHIK VESHIQPSLNQEDIYITTESLTTAAGRPGTGEHVPGS EMPVPDYTSIHIVQSPQGLILNATALPLPDKEFLSSCGYVSTDQLNKIMP (SEQ ID NO: 44), or a sequence having at least 80% identity thereto.

73. The chimeric receptor of claim 72, wherein the signaling region derived from GHR is encoded by the nucleotide sequence of AAGCAGCAGCGGATCAAGATGCTGATCCTGCCTCCTGTGCCTGTGCCTAAGATCAAG GGCATCGACCCCGACCTGCTGAAAGAGGGCAAGCTGGAAGAAGTGAACACCATCCT GGCCATCCACGACAGCTACAAGCCCGAGTTCCACAGCGACGATAGCTGGGTCGAGT TCATCGAGCTGGACATCGACGAGCCCGACGAGAAAACCGAGGAAAGCGACACCGA CAGACTGCTGAGCAGCGACCACGAGAAGTCCCACTCTAACCTGGGCGTGAAGGATG GCGATAGCGGCAGAACAAGCTGCTGCGAGCCCGATATCCTGGAAACCGACTTCAAC GCCAACGACATCCACGAGGGCACCAGCGAAGTTGCCCAGCCTCAAAGACTGAAGGG CGAAGCCGATCTGCTGTGCCTGGACCAGAAGAACCAGAACAACAGCCCCTACCACG ACGCCTGTCCTGCTACACAGCAGCCTAGTGTGATCCAGGCCGAGAAGAACAAGCCC CAGCCTCTGCCTACAGAGGGCGCCGAATCTACACATCAGGCCGCTCACATCCAGCTG AGCAACCCTAGCAGCCTGAGCAACATCGACTTCTACGCCCAAGTGTCCGACATCAC ACCAGCCGGATCTGTGGTGCTGTCTCCCGGCCAGAAAAACAAGGCCGGCATGTCCC AGTGCGACATGCACCCTGAAATGGTGTCCCTGTGCCAAGAGAACTTCCTGATGGACA ACGCCTACTTCTGCGAGGCCGACGCCAAGAAATGCATCCCTGTGGCTCCCCACATCA AGGTGGAAAGCCACATTCAGCCCAGCCTGAATCAAGAGGATATCTACATCACCACC GAGAGCCTGACCACCGCTGCTGGTAGACCTGGAACAGGCGAGCATGTGCCTGGCTC TGAAATGCCCGTGCCTGACTACACCAGCATCCACATCGTGCAGAGCCCTCAGGGCCT GATCCTGAATGCTACAGCTCTGCCCCTGCCAGACAAAGAGTTCCTGTCTAGCTGCGG CTACGTGTCCACCGACCAGCTGAACAAGATCATGCCC (SEQ ID NO: 43), or a sequence having at least 80% identity thereto.

74. The chimeric receptor of any one of claims 71-73, wherein the signaling region derived from MyD88 comprises the amino acid sequence of LEAAGGPGAGSAAPVSSTSSLPLAALNMR VRRRLSLFLNVRTQVAADWTALAEEMDFE YLEIRQLETQADPTGRLLDAWQGRPGASVGRLLELLTKLGRDDVLLELGPSIEEDCQKYI LKQQQEEAEKPLQVAAVDSSVPRTAELAGITTLDDPLGHMPERFDAFICYCPSDI (SEQ ID NO: 38), or AAGGPGAGSAAPVSSTSSLPLAALNMRVRRRLSLFLNVRTQVAADWTALAEEMDFEYL EIRQLETQADPTGRLLDAWQGRPGASVGRLLELLTKLGRDDVLLELGPSIEEDCQKYILK QQQEEAEKPLQVAAVDSSVPRTAELAGITTLDDPLGHMPERFDAFICYCPSDI (SEQ ID NO: 220), or a sequence having at least 80% identity thereto.

75. The chimeric receptor of claim 74, wherein the signaling region derived from MyD88 is encoded by the nucleotide sequence of CTCGAGGCGGCTGGAGGGCCAGGAGCTGGCTCTGCCGCGCCGGTGTCTTCCACCAG TTCACTGCCTTTGGCGGCTCTTAACATGCGGGTCAGGCGAAGATTGAGTTTGTTTCTG AATGTTAGGACTCAAGTAGCTGCCGACTGGACGGCCCTTGCGGAAGAAATGGACTT CGAGTATCTAGAGATTCGCCAACTTGAAACCCAGGCAGATCCTACAGGCAGACTCC TCGATGCGTGGCAGGGTCGGCCTGGAGCATCTGTGGGAAGGTTGCTGGAACTGTTG ACTAAACTTGGTCGAGATGACGTACTGCTGGAATTGGGTCCAAGCATCGAAGAAGA TTGTCAAAAATACATCCTCAAGCAGCAACAAGAGGAAGCAGAGAAACCATTGCAAG TAGCGGCAGTAGACTCTAGCGTACCGCGCACTGCTGAGCTTGCTGGAATCACGACCC TTGACGATCCTCTCGGGCATATGCCAGAGCGGTTCGACGCATTCATATGTTATTGTC CAAGCGATATC (SEQ or ID NO: 37) GCGGCTGGAGGGCCAGGAGCTGGCTCTGCCGCGCCGGTGTCTTCCACCAGTTCACTG CCTTTGGCGGCTCTTAACATGCGGGTCAGGCGAAGATTGAGTTTGTTTCTGAATGTT AGGACTCAAGTAGCTGCCGACTGGACGGCCCTTGCGGAAGAAATGGACTTCGAGTA TCTaGAGATTCGCCAACTTGAAACCCAGGCaGATCCTACAGGCAGACTCCTCGATGC GTGGCAGGGTCGGCCTGGAGCATCTGTGGGAAGGTTGCTGGAACTGTTGACTAAAC TTGGTCGAGATGACGTACTGCTGGAATTGGGTCCAAGCATCGAAGAAGATTGTCAA AAATACATCCTCAAGCAGCAACAAGAGGAAGCAGAGAAACCATTGCAAGTAGCGG CAGTAGACTCTAGCGTACCGCGCACTGCTGAGCTTGCTGGAATCACGACCCTTGACG ATCCTCTCGGGCATATGCCAGAGCGGTTCGACGCATTCATATGTTATTGTCCAAGCG ATATC (SEQ ID NO: 219), or a sequence having at least 80% identity thereto.

76. The chimeric receptor of any one of claims 71-75, wherein the transmembrane region derived from GHR comprises the amino acid sequence of FPWLLIIIFGIFGLTVMLFVFLFS (SEQ ID NO: 52), PWLLIIIFGIFGLTVMLFVFLFS (SEQ ID NO: 134), WLLIIIFGIFGLTVMLFVFLFS (SEQ ID NO: 136), LLIIIFGIFGLTVMLFVFLFS (SEQ ID NO: 138), LIIIFGIFGLTVMLFVFLFS (SEQ ID NO: 140), IIIFGIFGLTVMLFVFLFS (SEQ ID NO: 142), or IIFGIFGLTVMLFVFLFS (SEQ ID NO: 144), or a sequence having at least 80% identity thereto.

77. The chimeric receptor of claim 76, wherein the transmembrane region derived from GHR is encoded by the nucleotide sequence of
(SEQ ID NO: 133)
TTCCCCTGGCTGCTGATCATCATCTTCGGCATCTTTGGCCTGACCGTGA
TGCTGTTCGTGTTCCTGTTCAGC,
(SEQ ID NO: 135)
CCCTGGCTGCTGATCATCATCTTCGGCATCTTTGGCCTGACCGTGATGC
TGTTCGTGTTCCTGTTCAGC,
(SEQ ID NO: 137)
TGGCTGCTGATCATCATCTTCGGCATCTTTGGCCTGACCGTGATGCTGT
TCGTGTTCCTGTTCAGC,
(SEQ ID NO: 139)
CTGCTGATCATCATCTTCGGCATCTTTGGCCTGACCGTGATGCTGTTCG
TGTTCCTGTTCAGC,
(SEQ ID NO: 141)
CTGATCATCATCTTCGGCATCTTTGGCCTGACCGTGATGCTGTTCGTGT
TCCTGTTCAGC,
(SEQ ID NO: 143)
ATCATCATCTTCGGCATCTTTGGCCTGACCGTGATGCTGTTCGTGTTCC
TGTTCAGC,
or
(SEQ ID NO: 145)
ATCATCTTCGGCATCTTTGGCCTGACCGTGATGCTGTTCGTGTTCCTGT
TCAGC.

78. The chimeric receptor of any one of claims 71-77, wherein each polypeptide monomer comprises amino acids 28-611 of the amino acid sequence of SEQ ID NO: 221, or a sequence having at least 80% identity thereto.

79. The chimeric receptor of claim 78, wherein each polypeptide monomer is encoded by nucleotides 82-1833 of the nucleotide sequence of SEQ ID NO: 222, or a sequence having at least 80% identity thereto.

80. The chimeric receptor of any one of claims 1-79, wherein each polypeptide monomer further comprises a leader sequence.

81. The chimeric receptor of claim 80, wherein the leader sequence is derived from an immunoglobulin heavy chain variable region or colony stimulating factor 2 receptor alpha chain (CSF2RA).

82. The chimeric receptor of claim 81, wherein the leader sequence derived from an immunoglobulin heavy chain variable region comprises the amino acid sequence MDWIWRILFLVGAATGAHS (SEQ ID NO: 7).

83. The chimeric receptor of any one of claims 1-82, wherein each polypeptide monomer further comprises a hinge region.

84. The chimeric receptor of claim 83, wherein the hinge region is derived from the same cell-surface receptor as the signaling region.

85. The chimeric receptor of claim 83, wherein the hinge regions is derived from IgG1, IgG2, IgG3, IgG4, CD28, or CD8α.

86. The chimeric receptor of any one of claims 1-85, wherein the polypeptide monomer further comprises one or more additional polypeptide sequences.

87. The chimeric receptor of claim 86, wherein the one or more additional polypeptide sequences comprise are selected from one or more cellular markers, epitope tags, cytokines, safety switches, dimerization moieties, and degradation moieties.

88. A polynucleotide encoding the chimeric receptor of any one of claims 1-87.

89. The polynucleotide of claim 88, wherein the nucleotide sequence(s) is expressed in an inducible fashion, achieved with an inducible promoter, an inducible expression system, an artificial signaling circuit, and/or drug induced splicing.

90. The polynucleotide of claim 88 or 89, wherein the nucleotide sequence is operably linked to a promoter.

91. The polynucleotide of claim 90, wherein the promoter is an inducible promoter.

92. The polynucleotide of claim 90 or 91, wherein the promoter is a T cell-specific promoter or an NK cell-specific promoter.

93. The polynucleotide of any one of claims 88-92, further comprising one or more additional nucleotide sequences encoding one or more additional polypeptide sequences.

94. The polynucleotide of claim 93, wherein the one or more additional polypeptide sequences are selected from one or more cellular markers, epitope tags, cytokines, safety switches, dimerization moieties, and degradation moieties.

95. The polynucleotide of claim 94, wherein the epitope tag is FLAG or Myc.

96. The polynucleotide of claim 94, wherein the cellular marker is mClover3 or mRuby.

97. The polynucleotide of any one of claims 94-96, wherein the nucleotide sequence encoding the chimeric receptor is operably linked to the one or more additional nucleotide sequences encoding one or more additional polypeptide sequences via a sequence encoding a self-cleaving peptide and/or an internal ribosomal entry site (IRES).

98. The polynucleotide of claim 97, wherein the self-cleaving peptide is a 2A peptide.

99. The polynucleotide of claim 98, wherein the 2A peptide is T2A, P2A, E2A, or F2A peptide.

100. The polynucleotide of claim 98 or 99, wherein the 2A peptide is a P2A peptide.

101. The polynucleotide of claim 100, wherein the P2A peptide comprises the amino acid sequence GSGATNFSLLKQAGDVEENPGP (SEQ ID NO: 11), or an amino acid sequence having at least 80% sequence identity thereof.

102. The polynucleotide of any one of claims 88-101 which is a DNA molecule.

103. The polynucleotide of any one of claims 88-101 which is an RNA molecule.

104. A recombinant vector comprising the polynucleotide of any one of claims 88-103.

105. The recombinant vector of claim 104, wherein the vector is a viral vector.

106. The recombinant vector of claim 105, wherein the viral vector is a retroviral vector, a lentiviral vector, an adenoviral vector, an adeno-associated virus vector, an alphaviral vector, a herpes virus vector, a baculoviral vector, or a vaccinia virus vector.

107. The recombinant vector of claim 106, wherein the viral vector is a retroviral vector.

108. The recombinant vector of claim 104, wherein the vector is a non-viral vector.

109. The recombinant vector of claim 108, wherein the non-viral vector is a minicircle plasmid, a Sleeping Beauty transposon, a piggyBac transposon, or a single or double stranded DNA molecule that is used as a template for homology directed repair (HDR) based gene editing.

110. An isolated host cell comprising the polynucleotide of any one of claims 88-103 or the recombinant vector of any one of claims 104-109.

111. An isolated host cell comprising a chimeric receptor of any one of claims 1-87.

112. The isolated host cell of claim 110 or claim 111, wherein the host cell is an immune cell.

113. The isolated host cell of any one of claims 110-112, wherein the host cell is a T cell, a natural killer (NK) cell, a mesenchymal stem cell (MSC), or a macrophage.

114. The isolated host cell of any one of claims 110-113, wherein the host cell is a T cell.

115. The isolated host cell of claim 114, wherein the host cell is an αβ T cell receptor (TCR) T cell, a γδ T cell, a CD8+ T cell, a CD4+ T cell, a cytotoxic T cell, an invariant natural killer T (iNKT) cell, a memory T cell, a memory stem T cell (T_{SCM}), a naïve T cell, an effector T cell, a T-helper cell, or a regulatory T cell (Treg).

116. The isolated host cell of any one of claims 110-113, wherein the host cell is a NK cell.

117. The isolated host cell of claim 116, wherein the host cell is a NK cell derived from peripheral, cord blood, induced pluripotent stem (iPS) cells (iPSCs), and/or a cell line.

118. The isolated host cell of any one of claims 110-117, wherein the host cell further expresses one or more antigen-recognition molecules.

119. The isolated host cell of claim 118, wherein the one or more antigen-recognition molecules are selected from chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

120. The isolated host cell of any one of claims 110-119, wherein the host cell is further genetically modified to enhance its function by expressing one or more additional genes or deleting one or more inhibitory genes with a gene editing technology.

121. The isolated host cell of claim 120, wherein the one or more additional genes are selected from one or more transcription factors.

122. The isolated host cell of claim 121, wherein the transcription factor is c-Jun.

123. The isolated host cell of claim 120, wherein the one or more inhibitory genes is REGNASE-1 or DNMT3A.

124. The isolated host cell of claim 120, wherein the gene editing technology is CRISPR-Cas9 or transcription activator-like effector nuclease (TALEN).

125. The isolated host cell of any one of claims 110-124, wherein the host cell has been activated and/or expanded ex vivo.

126. The isolated host cell of any one of claims 110-125, wherein the host cell is an allogeneic cell.

127. The isolated host cell of any one of claims 110-125, wherein the host cell is an autologous cell.

128. The isolated host cell of any one of claims 112-127, wherein the immune cell is derived from an induced pluripotent stem (iPS) cell.

129. A pharmaceutical composition comprising the host cell of any one of claims 110-128 and a pharmaceutically acceptable carrier and/or excipient.

130. A method of enhancing an effector function of an immune cell, wherein the immune cell expresses one or more antigen-recognition molecules comprising genetically modifying the cell with the polynucleotide of any one of claims 88-103 or the recombinant vector of any one of claims 104-109.

131. The method of claim 130, wherein the one or more antigen-recognition molecules are selected from chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

132. The method of claim 130 or 131, wherein the effector function is one or more of expansion, persistence, and/or cytotoxicity.

133. A method of generating the isolated host cell of any one of claims 110-128, said method comprising genetically modifying the host cell with the polynucleotide of any one of claims 88-103 or the recombinant vector of any one of claims 104-109.

134. The method of claim 133, further comprising genetically modifying the host cell to express one or more antigen-recognition molecules.

135. The method of claim 134, wherein the one or more antigen-recognition molecules are selected from chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

136. The method of claim 133 or 134, wherein the genetic modifying step is conducted via viral gene delivery.

137. The method of claim 133 or 134, wherein the genetic modifying step is conducted via non-viral gene delivery.

138. The method of any one of claims 133-137, wherein the genetically modifying step is conducted ex vivo.

139. The method of any one of claims 133-138, wherein the method further comprises activation and/or expansion of the host cell ex vivo before, after, and/or during said genetic modification.

140. A method of treating a disease comprising administering to a subject an effective amount of the host cell of any one of claims 110-128, or the pharmaceutical composition of claim 129.

141. The method of any one of claim 140, said method comprising:
a) isolating T cells or NK cells from the subject or donor;
b) modifying said T cells or NK cells ex vivo with the polynucleotide of any one of claims 88-103 or the recombinant vector of any one of claims 104-109;
c) optionally, modifying said T cells or NK cells ex vivo to express one or more antigen-recognition molecules that bind an antigen associated with said disease;
d) optionally, expanding and/or activating the modified T cells or NK cells before, after, and/or during step b) or c); and
e) introducing a therapeutically effective amount of the modified T cells or NK cells into the subject.

142. The method of claim 141, wherein the one or more antigen-recognition molecules are selected from chimeric antigen receptors (CARs), T cell receptor fusion constructs (TRuCs), HLA-independent T cell receptors (HITs), synthetic T cell receptor and antigen receptor (STARs), T cell antigen couplers (TACs), bispecific T cell engagers, native or transgenic T cell receptors, and antibodies, or a combination thereof.

143. The method of any one of claims 140-142, wherein the disease is a cancer, infection, or autoimmune disease.

144. The method of any one of claims 140-143, wherein the subject is human.

145. A chimeric receptor which is a homodimer comprising two polypeptide monomers, wherein each polypeptide monomer comprises:
i) an extracellular region comprising a means for homodimerizing the two polypeptide monomers;
ii) a transmembrane region comprising a means for joining the intracellular regions with extracellular region; and
iii) an intracellular region comprising a means for enhancing effector function of an immune cell.

146. The chimeric receptor of claim 145, wherein the means for homodimerizing the two polypeptide monomers comprises at least one homodimerizing leucine zipper motif, at least one ligand-dependent homodimerizing motif, or at least one single chain variable fragment, or combinations thereof.

147. The chimeric receptor of claim 145 or 146, wherein the means for homodimerizing the two polypeptide monomers comprises at least one homodimerizing leucine zipper motif.

148. The chimeric receptor of claim 147, wherein the homodimerizing leucine zipper motif comprises at least five heptad repeats of amino acids with a leucine at every seventh position.

149. The chimeric receptor of any one of claims 146-148, wherein the homodimerizing leucine zipper motif is derived from transcription factor c-Jun.

150. The chimeric receptor of claim 149, wherein the homodimerizing leucine zipper motif comprises the amino acid sequence of IARLEEKVKTLKAQNSELASTANMLREQVAQLKQKVMNH (SEQ ID NO: 9), or a sequence having at least 80% identity thereto.

151. The chimeric receptor of claim 150, wherein the homodimerizing leucine zipper motif is encoded by the nucleotide sequence of ATCGCCAGGCTGGAGGAGAAGGTGAAGACCCTGAAGGCCCAGAACAGCGAGCTGG CCAGCACCGCCAACATGCTGAGGGAGCAGGTGGCCCAGCTGAAGCAGAAGGTGATG AACCAC (SEQ ID NO: 3), or a sequence having at least 80% identity thereto.

152. The chimeric receptor of claim 145, wherein the ligand-dependent homodimerizing motif undergoes homodimerization in the presence of a chemical inducer.

153. The chimeric receptor of any one of claims 145-152, wherein the means for enhancing effector function comprises at least one signaling region derived from a cell-surface receptor or a signal transducing adaptor protein, or a portion or variant thereof.

154. The chimeric receptor of claim 153, wherein the means for enhancing effector function comprises a signaling region derived from EpoR, or a portion or variant thereof.

155. The chimeric receptor of claim 153, wherein the means for enhancing effector function comprises a signaling region derived from GHR, or a portion or variant thereof.

156. The chimeric receptor of claim 153, wherein the means for enhancing effector function comprises a signaling region derived from MyD88, or a portion or variant thereof.

157. The chimeric receptor of claim 153, wherein the means for enhancing effector function comprises a signaling region derived from GHR, or a portion or variant thereof, and a signaling region derived from MyD88, or a portion or variant thereof.

158. A chimeric receptor which is a homodimer comprising two polypeptide monomers, wherein each polypeptide monomer comprises:
i) an extracellular region comprising a means for homodimerizing the two polypeptide monomers;
ii) a transmembrane region; and
iii) an intracellular region comprising at least one signaling region derived from a cell-surface receptor or a signal transducing adaptor protein, or a portion or variant thereof.

159. A chimeric receptor which is a homodimer comprising two polypeptide monomers, wherein each polypeptide monomer comprises:
i) an extracellular region comprising at least one homodimerizing motif;
ii) a transmembrane region; and
iii) an intracellular region comprising a means for enhancing effector function of an immune cell.

160. A chimeric receptor comprising i) an extracellular region; ii) a transmembrane region; and iii) an intracellular region comprising at least one signaling region derived from a cell-surface receptor or a signal transducing adaptor protein, or a portion or variant thereof, the improvement comprising said extracellular region comprising at least one homodimerizing motif.

161. A chimeric receptor, the improvement comprising two transmembrane polypeptide monomers each comprising i) an extracellular region comprising at least one homodimerizing motif and ii) an intracellular region comprising at least one signaling region derived from a cell-surface receptor or a signal transducing adaptor protein, or a portion or variant thereof.

162. The chimeric receptor of claim 160 or 161, wherein the improvement comprises said extracellular region comprising at least one homodimerizing leucine zipper motif, at least one ligand-dependent homodimerizing motif, or at least one single chain variable fragment, or combinations thereof.

163. The chimeric receptor of any one of claims 160-162, wherein the improvement comprises said extracellular region comprising at least one homodimerizing leucine zipper motif.

164. The chimeric receptor of claim 163, wherein the improvement comprises said homodimerizing leucine zipper motif comprising at least five heptad repeats of amino acids with a leucine at every seventh position.

165. The chimeric receptor of any one of claims 162-164, wherein the improvement comprises said homodimerizing leucine zipper motif being derived from transcription factor c-Jun.

166. The chimeric receptor of claim 165, wherein the improvement comprises said homodimerizing leucine zipper motif comprising the amino acid sequence of IARLEEKVKTLKAQNSELASTANMLREQVAQLKQKVMNH (SEQ ID NO: 9), or a sequence having at least 80% identity thereto.

167. The chimeric receptor of claim 165, wherein the improvement comprises said homodimerizing leucine zipper motif being encoded by the nucleotide sequence of ATCGCCAGGCTGGAGGAGAAGGTGAAGACCCTGAAGGCCCAGAACAGCGAGCTGG CCAGCACCGCCAACATGCTGAGGGAGCAGGTGGCCCAGCTGAAGCAGAAGGTGATG AACCAC (SEQ ID NO: 3), or a sequence having at least 80% identity thereto.

168. The chimeric receptor of claim 162, wherein the improvement comprises said ligand-dependent homodimerizing motif undergoing homodimerization in the presence of a chemical inducer.

169. The chimeric receptor of any one of claims 160-168, wherein the improvement further comprises said intracellular region comprising a signaling region derived from EpoR, or a portion or variant thereof.

170. The chimeric receptor of any one of claims 160-168, wherein the improvement further comprises said intracellular region comprising a signaling region derived from GHR, or a portion or variant thereof.

171. The chimeric receptor of any one of claims 160-168, wherein the improvement further comprises said intracellular region comprising a signaling region derived from MyD88, or a portion or variant thereof.

172. The chimeric receptor of any one of claims 160-168, wherein the improvement further comprises said intracellular region comprising a signaling region derived from GHR, or a portion or variant thereof, and a signaling region derived from MyD88, or a portion or variant thereof.

173. A polynucleotide encoding the chimeric receptor of any one of claims 160-172.

174. A recombinant vector comprising the polynucleotide of claim 173.

175. An immune cell expressing the chimeric receptor of any one of claims 160-172.

176. An immune cell comprising the polynucleotide of claim 173 or the recombinant vector of claim 174.

177. In a method of enhancing an effector function of an immune cell, the improvement comprising the immune cell expressing the chimeric receptor of any one of claims 160-172.

178. In a method of improving expansion or persistence or cytotoxicity of an antigen-activated immune cell, the improvement comprising the immune cell expressing the chimeric receptor of any one of claims 160-172.

179. In a method of treating a disease comprising administering to a subject an effective amount of an immune cell, the improvement comprising the immune cell expressing the chimeric receptor of any one of claims 160-172.
